(19) United States
(12) Patent Application Publication
(10) Pub. No.: US 2026/0284080 A1
(43) Pub. Date: Sep. 24, 2026

(54) NIRAPARIB AND ABIRATERONE ACETATE PLUS PREDNISONE TO IMPROVE CLINICAL OUTCOMES IN PATIENTS WITH METASTATIC CASTRATION-SENSITIVE PROSTATE CANCER AND HRR ALTERATIONS

(71) Applicant: Janssen Pharmaceutica NV, Beerse (BE)

(72) Inventors: Susan Xuemei Li, Lansdale, PA (US); Benoit Christian Thierry Baron, Leiden (NL); Shiva Dibaj, San Marcos, CA (US); Sharon Anne Mc Carthy, Shrewsbury, NJ (US); Angela Mennicke Lopez-Gitlitz, Los Angeles, CA (US); Won Kim, Burlingame, CA (US); Carly Rachel Varela, Burke, VA (US); Usha Singh, Bridgewater, NJ (US); Karen Ann Urtishak, Media, PA (US); Ke Zhang, San Diego, CA (US); Adam A. Del Corral, Upper Saddle River, NJ (US)

(21) Appl. No.: 19/556,487

(22) Filed: Mar. 4, 2026

Related U.S. Application Data
(60) Provisional application No. 63/767,386, filed on Mar. 5, 2025.
(60) Provisional application No. 63/939,716, filed on Dec. 12, 2025.
(60) Provisional application No. 63/971,921, filed on Jan. 30, 2026.

(51) Int. Cl. A61K 31/58; A61K 31/454; A61K 31/573; A61K 45/06; A61P 35/04

(52) U.S. Cl. CPC A61K 31/58; A61K 31/454; A61K 31/573; A61K 45/06; A61P 35/04

(57) ABSTRACT

The present disclosure relates to niraparib and abiraterone acetate, plus prednisone or prednisolone; for use in a method of improving the efficacy of treatment of metastatic castration-sensitive prostate cancer (mCSPC) in a patient with DNA-repair anomalies, in particular for improving the median radiographic progression-free survival (rPFS).

### CROSS-REFERENCE TO RELATED APPLICATIONS

The present application claims priority to and the benefit of U.S. Provisional Patent Application No. 63/767,386, filed Mar. 5, 2025; U.S. Provisional Patent Application No. 63/939,716, filed Dec. 12, 2025; and States Provisional Patent Application No. 63/971,921, filed Jan. 30, 2026. All foregoing United applications are incorporated herein by reference in their entireties for any and all purposes.

### TECHNICAL FIELD OF THE INVENTION

The present disclosure relates to products and methods for improving clinical outcomes in patients having metastatic castration-sensitive prostate cancer (mCSPC) and HRR gene alterations.

### BACKGROUND OF THE INVENTION

Worldwide, prostate cancer is the second most common cancer and the fifth leading cause of cancer death in men, accounting for 1.5 million new cancer cases and 400,000 cancer deaths in 2024. While patients with localized prostate cancer may be cured with current therapies, development of metastases heralds a lethal disease. Despite advances in the treatment of men with mCSPC, the 5-year survival rates are between 30% to 50%.

Metastatic castration-sensitive prostate cancer (mCSPC), also known as metastatic hormone-sensitive prostate cancer (mHSPC), presents significant challenges in treatment. For many years, the standard approach to manage this disease has been androgen deprivation therapy (ADT). This treatment lowers the levels of male hormones, such as testosterone, which prostate cancer cells often need to grow. While ADT can be effective for some patients, it is not a complete solution. The cancer can still progress, leading to a situation where patients may feel as though they are in a constant race against time.

Recently, new medications have been developed, including androgen receptor inhibitors and chemotherapy options, which aim to improve outcomes for patients with mCSPC. However, even with these advancements, there remains a critical gap in the available treatment options, particularly for patients who have specific genetic alterations known as homologous recombination repair (HRR) alterations. These changes can make mCSPC more difficult to treat, and current therapies often do not provide adequate control of the disease for these individuals. As a result, many patients with HRR alterations continue to face challenges, as standard treatments may not be effective for them.

There is thus an unmet clinical need for the treatment of HRR+ mCSPC patients.

### SUMMARY OF THE INVENTION

An objective of the present invention is to improve the efficacy of treatment of mCSPC with HRR gene alterations, in a patient.

An objective of the present invention is to improve the efficacy of treatment of mCSPC with HRR gene alterations, when compared to a once-daily oral dose of 1000 mg of abiraterone acetate and a once-daily oral dose of 10 mg of prednisone or prednisolone

An objective of the present invention is to improve the radiographic progression-free survival (rPFS) in a patient with mCSPC whose tumors harbor HRR gene alterations.

An objective of the present invention is to delay time to symptomatic progression (TSP) in a patient with mCSPC whose tumors harbor HRR gene alterations.

An objective of the present invention is to improve the overall survival (OS) in a patient with mCSPC whose tumors harbor HRR gene alterations, and with a manageable safety profile.

An objective of the present invention is to delay the time to subsequent therapy (TST) in a patient with mCSPC whose tumors harbor HRR gene alterations.

An objective of the present invention is to improve the treatment of mCSPC with HRR gene alterations, in a patient, while having minimal pain burden and a generally positive health-related quality of life (HRQOL).

### FIGURES

FIG. 1: Kaplan-Meier plot of rPFS assessed by investigator in BRCA patients. There is a statistically significant and clinically meaningful benefit in rPFS for subjects with BRCA gene alterations in the nira+AAP arm (median not reached) compared to those in the placebo+AAP arm (median: 26 months), with a 48% reduction in the risk of radiographic progression or death with nira+AAP treatment (stratified 2-sided log-rank p-value of <0.0001, which is below the prespecified significance boundary of 0.05).

FIG. 2: Kaplan-Meier plot of rPFS assessed by investigator in HRR effectors (BRCA1, BRCA2, BRIP1, PALB2, RAD51B, RAD54L). There is a statistically significant and clinically meaningful benefit in rPFS for subjects with HRR Effectors gene alterations in the nira+AAP arm (median not reached) compared to those in the placebo+AAP arm (median: 27.6 months), with a 43% reduction in the risk of radiographic progression or death with nira+AAP treatment (stratified 2-sided log-rank p-value of 0.0003, which is below the prespecified significance boundary of 0.025).

FIG. 3: Kaplan-Meier plot of rPFS assessed by investigator in All HRR (BRCA1, BRCA2, BRIP1, CDK12, CHEK2, FANCA, PALB2, RAD51B, RAD54L). There was a statistically significant and clinically meaningful benefit in rPFS in the All HRR population in the nira+AAP arm (median not reached) compared to those in the placebo+AAP arm (median: 29.5 months), with a 37% reduction in the risk of radiographic progression or death with nira+AAP treatment (stratified 2-sided log-rank p-value of 0.0001, which is below the prespecified significance boundary of 0.02475).

FIG. 4: Kaplan-Meier plot of Time to Symptomatic Progression (TSP) in BRCA patients. A statistically significant and clinically meaningful prolongation in TSP was observed in the nira+AAP arm (median not reached) compared to the placebo+AAP arm (median not reached), with a 56% reduction in the risk of symptomatic progression with nira+AAP treatment with the stratified 2-sided log-rank p-value 0.0001 crossing the prespecified significance boundary of 0.03426 for IA1.

FIG. 5: Kaplan-Meier plot of TSP in HRR Effector patients. A statistically significant and clinically meaningful prolongation in TSP was observed in the nira+AAP arm (median not reached) compared to the placebo+AAP arm (median not reached), with a 51% reduction in the risk of symptomatic progression with nira+AAP treatment.

FIG. 6: Kaplan-Meier plot of TSP in All HRR patients. A statistically significant and clinically meaningful prolongation in TSP was observed in the nira+AAP arm (median not reached) compared to the placebo+AAP arm (median not reached), with a 50% reduction in the risk of symptomatic progression with nira+AAP treatment.

FIG. 7: Kaplan-Meier plot of Overall Survival in BRCA patients. Median OS was not reached in either arm. The difference between arms shows a strong trend of improvement in the nira+AAP arm compared to the placebo+AAP arm with a 25% reduction in the risk of death with nira+AAP treatment.

FIG. 8: Kaplan-Meier plot of Overall Survival in HRR Effectors patients. Median OS was not reached in either arm. The difference between arms shows a trend of improvement in the nira+AAP arm compared to the placebo+AAP arm with a 19% reduction in the risk of death with nira+AAP treatment.

FIG. 9: Kaplan-Meier plot of Overall Survival in All HRR population. Median OS was not reached in either arm. The difference between arms shows a trend of improvement in the nira+AAP arm compared to the placebo+AAP arm with a 21% reduction in the risk of death with nira+AAP treatment.

FIG. 10: Non-BRCA-Time to Symptomatic Progression

FIG. 11: Kaplan-Meier Plot of Radiographic Progression-Free Survival in the BRCA2m Population (AMPLITUDE)

FIG. 12: Kaplan-Meier Plot of BICR Assessed Radiographic Progression-Free Survival in the BRCAm Population (MAGNITUDE, primary analysis)

FIG. 13: Kaplan-Meier Plot of Overall Survival in the BRCAm Population (MAGNITUDE, final analysis)

### DETAILED DESCRIPTION

The present inventions may be understood more readily by reference to the following detailed description, taken in connection with the accompanying examples, which form a part of this disclosure. It is to be understood that these inventions are not limited to the specific products, methods, conditions or parameters described and/or shown herein, and that the terminology used herein is for the purpose of describing embodiments by way of example only and is not intended to be limiting of the claimed inventions.

The entire disclosures of each patent, patent application, and publication cited or described in this document are hereby incorporated herein by reference.

### Definitions

As employed above and throughout the disclosure, the following terms and abbreviations, unless otherwise indicated, shall be understood to have the following meanings.

In the present disclosure the singular forms “a,”, “an,” and “the” include the plural reference, and reference to a given numerical value includes at least that value, unless the context clearly indicates otherwise. Thus, for example, a reference to “an ingredient” is a reference to one or more of such ingredients and equivalents thereof known to those skilled in the art, and so forth. Furthermore, when indicating that a certain element “may be” X, Y, or Z, it is not intended by such usage to exclude in all instances other choices for the element.

When values are expressed as approximations, by use of the antecedent “about,” it will be understood that the value forms another embodiment. As used herein, “about X” (where X is a numerical value) preferably refers to ±10% of the recited value, inclusive. For example, the phrase “about 8” refers to a value of 7.2 to 8.8, inclusive; as another example, the phrase “about 8%” refers to a value of 7.2% to 8.8%, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like. In addition, when a list of alternatives is positively provided, such a listing can also include embodiments where any of the alternatives may be excluded. For example, when a range of “1 to 5” is described, such a description can support situations whereby any of 1, 2, 3, 4, or 5 are excluded; thus, a recitation of “1 to 5” may support “1 and 3-5, but not 2”, or simply “wherein 2 is not included.”

The transitional terms “comprising,” “consisting essentially of,” and “consisting” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents), also provide, as embodiments, those which are independently described in terms of “consisting of and “consisting essentially of”.

When a list is presented, unless stated otherwise, it is understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

The term “cancer” as used herein refers to an abnormal growth of cells that tend to proliferate in an uncontrolled way and, in some cases, to metastasize (spread).

The term “prostate cancer” as used herein refers to histologically or cytologically confirmed adenocarcinoma of the prostate.

The term “androgen-deprivation therapy (ADT)” refers to medical or surgical castration. It is the first line of treatment against advanced prostate cancer and is also used as an adjuvant to local treatment of high-risk disease. Medical castration is accomplished using GnRH agonists or GnRH antagonists that act in the anterior pituitary gland to decrease the release of LH though down-regulation of GnRH receptors or by directly inhibiting GnRH receptors. Medical castration is also accomplished using antiandrogen therapy, to block the body's ability to use any androgens. Surgical castration is accomplished through orchiectomy. In both instances, testosterone levels are quickly reduced to castration levels, which is currently defined as serum testosterone levels of <50 ng/dl (1.7 nmol/L). Examples of common GnRH agonists/antagonists used for ADT include leuprolide, goserelin, triptorelin, buserelin, histrelin, abarelix degarelix, and relugolix. Examples of antiandrogens include cyproterone acetate, flutamide, nilutamide, bicalutamide, enzalutamide, abiraterone acetate, seviteronel, apalutamide, darolutamide, galeterone.

The term “CSPC” as used herein refers to castration-sensitive prostate cancer. CSPC is stage of prostate cancer in which the cancer cells are still responsive to hormonal therapy aimed at lowering testosterone levels, which is a key driver of prostate cancer growth. In CSPC, the disease is still sensitive to treatments like androgen deprivation therapy (ADT) and androgen receptor inhibitors. This contrasts with castration-resistant prostate cancer (CRPC), where the cancer progresses despite low levels of testosterone.

The term “metastatic castration-sensitive prostate cancer” or “mCSPC” refers to castration-sensitive prostate cancer that has spread beyond the prostate gland, i.e. it has metastasized to other parts of the human body.

The terms “treat,” “treating” and “treatment” refer to the eradication, removal, modification, management or control of a tumor or primary, regional, or metastatic cancer cells or tissue, in particular prostate cancer cells or tissue, and the minimization or delay of the spread of cancer, in particular prostate cancer. The minimization or delay of the spread of cancer includes inhibition of the progress of cancer, a reduction in the rate of progress of cancer, or a halt in the rate of progress of cancer.

The term “randomization” as it refers to a clinical trial refers to the time when the patient is confirmed eligible for the clinical trial and gets assigned to a treatment arm.

The terms “kit” and “article of manufacture” are used as synonyms.

The term “subject” and “patient” and “human” are used interchangeably. Usually the subject, patient, or human is a male subject, patient, or human.

The term “drug product” means a pharmaceutical formulation that comprises niraparib and abiraterone acetate. In one embodiment, the pharmaceutical formulation comprises niraparib tosylate monohydrate and abiraterone acetate.

The terms “sale” or “selling” means transferring title to a drug product, e.g., a pharmaceutical composition or an oral dosage form, in an arms-length transaction from a seller to a buyer.

The term “offering for sale” means the proposal of a sale by a seller to a buyer for a drug product, e.g., a pharmaceutical composition and an oral dosage form.

As used herein, unless otherwise defined, the term “clinically effective amount” means an amount of one or more active pharmaceutical ingredients that provides for the achievement of the prevention, delay of onset, or amelioration of symptoms of prostate cancer in a patient as confirmed by efficacy data secured through a clinical trial.

The term “pharmaceutically acceptable” means that is generally safe, non-toxic and neither biologically nor otherwise undesirable and includes that are acceptable for human pharmaceutical use.

The terms “formulation” and “composition” may be used interchangeably in the present disclosure. Both “formulation” and “composition” refer to combining two active pharmaceutical ingredients (APIs), either as fixed-dose combinations or as free-dose combinations. In other words, the “formulation” or “composition” refers to a two-drug combination. As such the term “a pharmaceutical formulation” refers to fixed-dose combinations and free-dose combinations. The two or more components encompass herein at least 1) abiraterone acetate; 2) niraparib, and any pharmaceutically acceptable salt, solvate, and hydrate forms thereof, for example niraparib tosylate monohydrate; and 3) additional pharmaceutically acceptable components. The additional components include pharmaceutically acceptable carriers and excipients.

As used herein, a “fixed-dose combination” (FDC) are formulations or compositions that include abiraterone acetate and niraparib and any pharmaceutically acceptable salt, solvate, and hydrate forms thereof, for example niraparib tosylate monohydrate, in a single oral dosage form.

In contrast, a “free-dose combination” (FrDC) are formulations or compositions that include two or more active ingredients combined in separate dosage forms. For example, 1) a dosage form comprising abiraterone acetate; and 2) a separate dosage form comprising niraparib, and any pharmaceutically acceptable salt, solvate, and hydrate forms thereof, for example niraparib tosylate monohydrate.

The terms “excipient” and carrier” are used interchangeably in the present disclosure. The European Pharmacopoeia (Ph. Eur.) defines an excipient as “any component, other than the active substance(s), present in a medicinal product or used in the manufacture of the product. The intended function of an excipient is to act as the carrier (vehicle or basis) or as a component of the carrier of the active substance(s) and, in so doing, to contribute to product attributes such as stability, biopharmaceutical profile, appearance and patient acceptability and to the ease with which the product can be manufactured. Usually, more than one excipient is used in the formulation of a medicinal product.” The terms vehicle and basis are further defined in the same pharmacopoeia: “A vehicle is the carrier, composed of one or more excipients, for the active substance(s) in a liquid preparation” and “A basis is the carrier, composed of one or more excipients, for the active substance(s) in semi-solid and solid preparations.”

Radiographic progression-free survival (rPFS) is defined as the time interval from the date of randomization to the first date of radiographic progression or death due to any cause, whichever occurs first. Radiographic progression is determined by first occurrence of progression by bone scan (according to PCWG3 criteria) or progression of soft tissue lesions by CT or MRI (according to RECIST 1.1 criteria).

Radiographic progression should be evaluated as follows:
Progression of soft tissue lesions measured by CT or MRI as defined by RECIST 1.1.
Progression by bone lesions observed by bone scan and based on PCWG3. Under these criteria, any bone progression must be confirmed by a subsequent scan ≥6 weeks later. The Week 8 scan (first post-treatment scan) should be used as the baseline to which all subsequent scans are compared to determine progression. Bone progression is defined as one of the following:
   1. Subject whose Week 8 scan is observed to have ≥2 new bone lesions would fall into one of the 2 categories below:
      a. Subject whose confirmatory scan (which is performed ≥6 weeks later) shows ≥2 new lesions compared to the Week 8 scan (ie, a total of ≥4 new lesions compared to baseline scan) will be considered to have bone scan progression at Week 8.
      b. Subject whose confirmatory scan did not show ≥2 new lesions compared to the Week 8 scan will not be considered to have bone scan progression. The Week 8 scan will be considered as the baseline scan to which subsequent scans are compared. The FIRST scan timepoint that shows ≥2 new lesions compared with the Week 8 scan will be considered as the bone scan progression timepoint if these new lesions are confirmed by a subsequent scan ≥6 weeks later.
   2. For a subject whose Week 8 scan does not have ≥2 new bone lesions compared to baseline scan, the FIRST scan timepoint that shows ≥2 new lesions compared with the Week 8 scan will be considered as the bone scan progression timepoint if these new lesions are confirmed by a subsequent scan ≥6 weeks later.

Subjects without radiographic progression or death will be censored at the last disease assessment date if they never start subsequent anti-cancer therapy or censored at the last disease assessment date prior to the start of the subsequent anti-cancer therapy if they started subsequent anti-cancer therapy. Key censoring rules are summarized below.

| Scenario | Censoring Rule |
|---|---|
| No disease assessment at baseline or | Censored on the date of randomization |
| No disease assessment after baseline | |
| Subjects who are lost to follow-up or withdraw | Censored on the date of the last disease |
| from study | assessment |
| Subjects who receive new systemic anticancer | Censored on the date of the last disease |
| therapy known or intended for the treatment of | assessment prior to the start of the new systemic |
| mCRPC during the study prior to documented | anti-cancer therapy |
| disease progression or death | |
| Subjects with no evidence of radiographic | Censored on the date of the last disease |
| progressive disease or death | assessment |
| Subjects who miss ≥2 consecutive planned | Censored on the date of the last disease |
| radiographic scans or has ≥2 consecutive | assessment before the missed/unevaluable scans |
| unevaluable scans before progression or death | |
| No postbaseline assessment and death occurred | Censored on the date of randomization |
| after missed 2 or more planned disease | |
| assessments | |

The term “overall survival” is defined as the time from randomization to the date of death due to any cause. Survival data for subjects who are alive at the time of the analysis was to be censored on the last known date that they were alive. In addition, for subjects with no post-baseline information survival, data was to be censored on the date of randomization; for subjects who are lost to follow-up or who withdraw consent, data is censored on the last known date that they were alive. Administration of a safe and effective amount of the two-drug combination of the present disclosure provides improved anti-tumor activity as measured by overall survival.

The term “time to symptomatic progression” is defined as the time from randomization to documentation in the Case Report Form (CRF) of any of the following (whichever occurs earlier): (1) development of a skeletal-related event (SRE): pathologic fracture, spinal cord compression, or need for surgical intervention or radiation therapy to the bone; (2) pain progression or worsening of disease-related symptoms requiring initiation of a new systemic anti-cancer therapy; or (3) development of clinically significant symptoms due to loco-regional tumor progression requiring surgical intervention or radiation therapy. In some embodiments, administration of a safe and effective amount of the two-drug combination of the invention provides improved anti-tumor activity as measured by time to symptomatic progression.

Time to symptomatic progression (TSP) is also defined as the need to initiate: External beam radiation therapy (EBRT) for skeletal symptoms, tumor-related orthopedic surgical intervention, other cancer-related procedures (for example: nephrostomy insertion, bladder catheter insertion, EBRT, or surgery for tumor symptoms other than skeletal), new systemic anti-cancer therapy because of cancer pain or having cancer-related morbid events (for example: fracture, symptomatic and/or pathologic, cord compression, urinary obstructive events).

The term “time to initiation of cytotoxic chemotherapy” or “time to cytotoxic chemotherapy” (TCC) is defined as the time from randomization to documentation of a new cytotoxic chemotherapy being administered to the subject (e.g., survival follow-up CRF). Time to initiation of cytotoxic chemotherapy for subjects who do not start a cytotoxic chemotherapy is censored on the date of last contact. In some embodiments, administration of a safe and effective amount of the two-drug combination of the disclosure provides improved anti-tumor activity as measured by time to cytotoxic chemotherapy.

The time to PSA progression is defined as the time from randomization to the first date of documented PSA progression per PCWG3 criteria. There will be a PSA progression when after decline from baseline: PSA increase ≥25% and ≥2 ng/ml above the nadir, and which is confirmed by a second value ≥3 weeks later (i.e., a confirmed rising trend); and when no decline from baseline: PSA increase ≥25% and ≥2 ng/mL from baseline beyond 12 weeks.

Subjects with no PSA progression at the time of analysis will be censored on the last known date with no progression. Subjects without a baseline PSA or without any post baseline values will be censored at randomization date.

PSA response rate is the proportion of subjects achieving a PSA decline of ≥50% and confirmed at 3-4 weeks later according to PCWG3 criteria by Week 12 and during treatment period.

Time-to-pain progression is defined as the time from date of randomization to the date of the first observation of pain progression. Pain progression is defined as an average increase by 2 points from baseline in the BPI-SF worst pain intensity (item 3) observed at 2 consecutive evaluations ≥3 weeks apart. Subjects with no pain progression at the time of analysis will be censored at last date of BPI-SF pain score collection.

Time to initiation of subsequent therapy is defined as the time from the date of randomization to the date of initiation of subsequent anticancer therapy for prostate cancer. Subjects who did not initiate subsequent anticancer therapy at the time of the analysis will be censored on last visit date prior to or on last known alive date. Subsequent anticancer therapy for prostate cancer will include categories of chemotherapy, hormone therapy, PARPi and any other kind of therapy for prostate cancer.

Objective response rate (ORR) is defined as the proportion of subjects with measurable disease whose best response is either complete response (CR) or partial response (PR) by BICR as defined by RECIST 1.1 with no evidence of bone progression according to the PCWG3 criteria.

Duration of response in subjects with measurable disease (based on modified RECIST 1.1) is defined from the time of documented response to the first date of documented disease progression. This endpoint considers only the subjects who (1) had a measurable lesion at baseline according to RECIST 1.1 (i.e., having a record in the Target dataset) and (2) had a tumor response of CR or PR post baseline and before pharmacodynamic (PD) identified by RECIST. For RECIST lesions, as the scan dates associated with a given visit may span more than a single date, PD date will be the earliest scan date for the visit; all other response will be linked to the latest scan date for the visit. Definition of PD and rule for censoring are the same as that for the rPFS by BICR.

Progression-free survival on first subsequent therapy (PFS2) is defined as time from randomization to the date of progression (radiographic, clinical, or PSA progression) on the first subsequent therapy or death from any cause, whichever occurs first. General rules for PFS2 event and censoring:
1. For subjects who initiated a subsequent anti-cancer therapy:
   a. If there is a disease progression on 1^{st} subsequent anti-cancer therapy or death, this is a PFS2 event, date of PFS2=minimum of disease progression date and death date.
   b. If no disease progression on 1^{st} subsequent anticancer therapy and no death prior to start of 2^{nd} subsequent anti-cancer therapy, this is not a PFS2 event, the subject will be censored at start date of 2^{nd} subsequent anti-cancer therapy-1 day.
   c. If no disease progression on 1^{st} subsequent anticancer therapy and no death and no start of 2^{nd} subsequent anti-cancer therapy, this is not a PFS2 event, the subject will be censored at last known alive date.
2. For subjects who did not receive any subsequent anti-cancer therapy:
   a. If a subject died, this is a PFS2 event with death date as date of PFS2.
   b. If a subject did not die, this is not a PFS2 event, the subject will be censored at last known alive date.

The term “PSA₅₀ response” as used herein means a decline of serum prostate-specific antigen by 50% from baseline.

The term “survival benefit” as used herein means an increase in survival of the patient from time of randomization on the trial of administered drug to death. In some embodiments, the survival benefit is about 1, about 2, about 3, about 4, about 5, about 6, about 7, about 8, about 9, about 10, about 15, about 20, about 25, about 30, about 35, about 40, about 45, about 50, about 55, about 60, about 80, about 100 months or greater than 100 months.

The term “delay in symptoms related to disease progression” as used herein means an increase in time in the development of symptoms such as pain, urinary obstruction, and quality of life considerations from the time of randomization on the trial of administered drug.

The term “trial drug” as used herein refers to single-daily oral dose of 200 mg niraparib, 1000 mg of abiraterone acetate, and a daily oral dose of 10 mg of prednisone or prednisolone.

The term “placebo” as used in the chapters “Detailed Description”, the “Figures”, and the “Examples” refers to 1000 mg of abiraterone acetate plus 10 mg of prednisone/prednisolone.

### Embodiments of the Invention

The present disclosure relates to a method of improving the median radiographic progression-free survival (rPFS) in a patient with metastatic castration-sensitive prostate cancer (mCSPC), who is positive for germline and/or somatic homologous recombination repair (HRR) gene alterations; said method comprising administering to said patient a daily dose of 200 mg of niraparib, 1000 mg of abiraterone acetate, and 5 mg of prednisone or prednisolone; wherein the HRR gene alterations are selected from the genes BRCA2 or CHEK2, and wherein the rPFS is improved as indicated by a hazard ratio of less than 0.60 compared to a treatment with 1000 mg of abiraterone acetate and 5 mg of prednisone or prednisolone.

The present disclosure further relates to the method of improving the median rPFS as described above, wherein the patient has a BRCA2 alteration and said median rPFS is improved as indicated by a hazard ratio of 0.451 compared to a treatment with 1000 mg of abiraterone acetate and 5 mg of prednisone or prednisolone.

The present disclosure further relates to the method of improving the median rPFS as described above, wherein the median rPFS is more than 41 months, compared to 25.6 months for a treatment with 1000 mg of abiraterone acetate and 5 mg of prednisone or prednisolone.

The present disclosure further relates to the method of improving the median rPFS as described above, wherein the patient has a CHEK2 alteration and said median rPFS is improved as indicated by a hazard ratio of 0.550 compared to a treatment with 1000 mg of abiraterone acetate and 5 mg of prednisone or prednisolone.

The present disclosure further relates to the method of improving the median rPFS as described above, wherein the median rPFS is 47.87 months, compared to 30.55 months for a treatment with 1000 mg of abiraterone acetate and 5 mg of prednisone or prednisolone.

The present disclosure relates to a method of improving the median time to symptomatic progression (TSP) in a patient with metastatic castration-sensitive prostate cancer (mCSPC), who is positive for germline and/or somatic HRR gene alterations; said method comprising administering to said patient a daily dose of 200 mg of niraparib, 1000 mg of abiraterone acetate, and 5 mg of prednisone or prednisolone; wherein the HRR gene alterations are selected from non-BRCA genes defined as single or co-occurring alterations in BRIP1, PALB2, RAD51B, and RAD54L and excluding those with BRCA co-occurring.

The present disclosure further relates to the method of improving the median TSP as described above, wherein said median TSP is improved as indicated by a hazard ratio of less than 0.60 compared to a treatment with 1000 mg of abiraterone acetate and 5 mg of prednisone or prednisolone, preferably wherein the TSP is improved as indicated by a hazard ratio of 0.58 (95% CI: 0.37, 0.93; nominal p-value=0.022).

The present disclosure relates to a method of improving the median overall survival (OS) in a patient with metastatic castration-sensitive prostate cancer (mCSPC), who is positive for germline and/or somatic BRCA2 gene alterations; said method comprising administering to said patient a daily dose of 200 mg of niraparib, 1000 mg of abiraterone acetate, and 5 mg of prednisone or prednisolone.

The present disclosure relates to a method of improving time to subsequent therapy (TST) in a patient with metastatic castration-sensitive prostate cancer (mCSPC), who is positive for germline and/or somatic HRR gene alterations; said method comprising administering to said patient a daily dose of 200 mg of niraparib, 1000 mg of abiraterone acetate, and 5 mg of prednisone or prednisolone; wherein the HRR gene alterations are selected from the genes BRCA1, BRCA2, BRIP1, CDK12, CHEK2, FANCA, PALB2, RAD51B, and RAD54L.

The present disclosure further relates to the method of improving the median TST as described above, wherein said TST is improved as indicated by a hazard ratio of 0.52 (95% CI: 0.40,0.66, nominal p<0.0001) compared to a treatment with 1000 mg of abiraterone acetate and 5 mg of prednisone or prednisolone.

The present disclosure relates to a method of improving time to subsequent therapy (TST) in a patient with metastatic castration-sensitive prostate cancer (mCSPC), who is positive for germline and/or somatic HRR gene alterations; said method comprising administering to said patient a daily dose of 200 mg of niraparib, 1000 mg of abiraterone acetate, and 5 mg of prednisone or prednisolone; wherein the HRR gene alterations are selected from the genes BRCA1, BRCA2, BRIP1, PALB2, RAD51B, and RAD54L.

The present disclosure further relates to the method of improving the median TST as described above, wherein said TST is improved as indicated by a hazard ratio of 0.471 (95% CI: 0.35,0.64, nominal p-value<0.0001). compared to a treatment with 1000 mg of abiraterone acetate and 5 mg of prednisone or prednisolone.

The present disclosure relates to a method of improving time to subsequent therapy (TST) in a patient with metastatic castration-sensitive prostate cancer (mCSPC), who is positive for germline and/or somatic HRR gene alterations; said method comprising administering to said patient a daily dose of 200 mg of niraparib, 1000 mg of abiraterone acetate, and 5 mg of prednisone or prednisolone; wherein the HRR gene alterations are selected from the genes BRCA1 and BRCA2.

The present disclosure further relates to a method of improving the TST as described above, wherein said TST is improved as indicated by a hazard ratio of 0.41 (95% CI: 0.30,0.57, nominal p<0.0001) compared to a treatment with 1000 mg of abiraterone acetate and 5 mg of prednisone or prednisolone.

The present disclosure relates to a method of improving the radiographic progression-free survival (rPFS) in a patient with metastatic castration-sensitive prostate cancer (mCSPC), who is positive for germline and/or somatic HRR gene alterations; said method comprising administering to said patient a daily dose of 200 mg of niraparib, 1000 mg of abiraterone acetate, and 5 mg of prednisone or prednisolone; wherein the HRR gene alterations are selected from the genes BRCA1 and BRCA2, and wherein the rPFS is improved as indicated by a hazard ratio of 0.49 (95% CI: 0.36, 0.67; p<0.0001) compared to a treatment with 1000 mg of abiraterone acetate and 5 mg of prednisone or prednisolone.

The present disclosure relates to a method of improving the radiographic progression-free survival (rPFS) in a patient with mCSPC, who is positive for germline and/or somatic HRR gene alterations; said method comprising administering to said patient a daily dose of 200 mg of niraparib, 1000 mg of abiraterone acetate, and 5 mg of prednisone or prednisolone; wherein the HRR gene alterations are selected from the genes BRCA1, BRCA2, BRIP1, PALB2, RAD51B, and RAD54L, and wherein the rPFS is improved as indicated by a hazard ratio of 0.58 (95% CI: 0.44, 0.76; p<0.001) compared to a treatment with 1000 mg of abiraterone acetate and 5 mg of prednisone or prednisolone.

The present disclosure relates to a method of improving the radiographic progression-free survival (PFS) in a patient with mCSPC, who is positive for germline and/or somatic HRR gene alterations; said method comprising administering to said patient a daily dose of 200 mg of niraparib, 1000 mg of abiraterone acetate, and 5 mg of prednisone or prednisolone; wherein the HRR gene alterations are selected from the genes BRCA1, BRCA2, BRIP1, CDK12, CHEK2, FANCA, PALB2, RAD51B, and RAD54L, and wherein the rPFS is improved as indicated by a hazard ratio of 0.62 (95% CI: 0.49, 0.77; p<0.0001) compared to a treatment with 1000 mg of abiraterone acetate and 5 mg of prednisone or prednisolone.

The present disclosure relates to a method of improving the radiographic progression-free survival (rPFS) in a patient with mCSPC, who is positive for germline and/or somatic HRR gene alterations; said method comprising administering to said patient a daily dose of 200 mg of niraparib, 1000 mg of abiraterone acetate, and 5 mg of prednisone or prednisolone; wherein the HRR gene alterations are selected from the genes BRIP1, PALB2, RAD51B, and RAD54L, excluding those with BRCA co-occurring, and wherein the rPFS is improved as indicated by a hazard ratio of 0.81 (95% CI: 0.58, 1.14; p=0.23) compared to a treatment with 1000 mg of abiraterone acetate and 5 mg of prednisone or prednisolone.

The present disclosure relates to a method of improving the time to symptomatic progression (TSP) in a patient with mCSPC, who is positive for germline and/or somatic HRR gene alterations; said method comprising administering to said patient a daily dose of 200 mg of niraparib, 1000 mg of abiraterone acetate, and 5 mg of prednisone or prednisolone; wherein the HRR gene alterations are selected from the genes BRCA1 and BRCA2, and wherein the TSP is improved as indicated by a hazard ratio of 0.46 (95% CI: 0.31, 0.69; p=0.0001) compared to a treatment with 1000 mg of abiraterone acetate and 5 mg of prednisone or prednisolone.

The present disclosure relates to a method of improving the time to symptomatic progression (TSP) in a patient with mCSPC, who is positive for germline and/or somatic HRR gene alterations; said method comprising administering to said patient a daily dose of 200 mg of niraparib, 1000 mg of abiraterone acetate, and 5 mg of prednisone or prednisolone; wherein the HRR gene alterations are selected from the genes BRCA1, BRCA2, BRIP1, PALB2, RAD51B, and RAD54L, and wherein the TSP is improved as indicated by a hazard ratio of 0.52 (95% CI: 0.35, 0.75; p=0.0004) compared to a treatment with 1000 mg of abiraterone acetate and 5 mg of prednisone or prednisolone.

The present disclosure relates to a method of improving the time to symptomatic progression (TSP) in a patient with mCSPC, who is positive for germline and/or somatic HRR gene alterations; said method comprising administering to said patient a daily dose of 200 mg of niraparib, 1000 mg of abiraterone acetate, and 5 mg of prednisone or prednisolone; wherein the HRR gene alterations are selected from the genes BRCA1, BRCA2, BRIP1, CDK12, CHEK2, FANCA, PALB2, RAD51B, and RAD54L, and wherein the TSP is improved as indicated by a hazard ratio of 0.51 (95% CI: 0.38, 0.69; p<0.0001) compared to a treatment with 1000 mg of abiraterone acetate and 5 mg of prednisone or prednisolone.

The present disclosure relates to a method of improving the time to symptomatic progression (TSP) in a patient with mCSPC, who is positive for germline and/or somatic HRR gene alterations; said method comprising administering to said patient a daily dose of 200 mg of niraparib, 1000 mg of abiraterone acetate, and 5 mg of prednisone or prednisolone; wherein the HRR gene alterations are selected from the genes BRIP1, PALB2, RAD51B, and RAD54L, excluding those with BRCA co-occurring, and wherein the TSP is improved as indicated by a hazard ratio of 0.58 (95% CI: 0.37, 0.93; p=0.022) compared to a treatment with 1000 mg of abiraterone acetate and 5 mg of prednisone or prednisolone.

The present disclosure relates to a method of improving the time to subsequent therapy (TST) in a patient with mCSPC, who is positive for germline and/or somatic HRR gene alterations; said method comprising administering to said patient a daily dose of 200 mg of niraparib, 1000 mg of abiraterone acetate, and 5 mg of prednisone or prednisolone; wherein the HRR gene alterations are selected from the genes BRCA1 and BRCA2, and wherein the TST is improved as indicated by a hazard ratio of 0.41 (95% CI: 0.30, 0.57; p<0.0001) compared to a treatment with 1000 mg of abiraterone acetate and 5 mg of prednisone or prednisolone.

The present disclosure relates to a method of improving the time to subsequent therapy (TST) in a patient with mCSPC, who is positive for germline and/or somatic HRR gene alterations; said method comprising administering to said patient a daily dose of 200 mg of niraparib, 1000 mg of abiraterone acetate, and 5 mg of prednisone or prednisolone; wherein the HRR gene alterations are selected from the genes BRCA1, BRCA2, BRIP1, PALB2, RAD51B, and RAD54L, and wherein the TST is improved as indicated by a hazard ratio of 0.47 (95% CI: 0.35, 0.64; p<0.0001) compared to a treatment with 1000 mg of abiraterone acetate and 5 mg of prednisone or prednisolone.

The present disclosure relates to a method of improving the time to subsequent therapy (TST) in a patient with mCSPC, who is positive for germline and/or somatic HRR gene alterations; said method comprising administering to said patient a daily dose of 200 mg of niraparib, 1000 mg of abiraterone acetate, and 5 mg of prednisone or prednisolone; wherein the HRR gene alterations are selected from the genes BRCA1, BRCA2, BRIP1, CDK12, CHEK2, FANCA, PALB2, RAD51B, and RAD54L, and wherein the TST is improved as indicated by a hazard ratio of 0.52 (95% CI: 0.40, 0.66; p<0.0001) compared to a treatment with 1000 mg of abiraterone acetate and 5 mg of prednisone or prednisolone.

The present disclosure relates to a method of improving the time to subsequent therapy (TST) in a patient with mCSPC, who is positive for germline and/or somatic HRR gene alterations; said method comprising administering to said patient a daily dose of 200 mg of niraparib, 1000 mg of abiraterone acetate, and 5 mg of prednisone or prednisolone; wherein the HRR gene alterations are selected from the genes BRIP1, PALB2, RAD51B, and RAD54L, excluding those with BRCA co-occurring, and wherein the TST is improved as indicated by a hazard ratio of 0.73 (95% CI: 0.50, 1.08; p=0.12) compared to a treatment with 1000 mg of abiraterone acetate and 5 mg of prednisone or prednisolone.

The present disclosure relates to a method of improving the median radiographic progression-free survival (rPFS), the median time to symptomatic progression (TSP), the median overall survival (OS), or the time to subsequent therapy (TST), in a patient with metastatic castration-sensitive prostate cancer (mCSPC), who is positive for germline and/or somatic homologous recombination repair (HRR) gene alterations; said method comprising administering to said patient a daily dose of 200 mg of niraparib, 1000 mg of abiraterone acetate, and 5 mg of prednisone or prednisolone; wherein the HRR gene alterations are selected from the genes BRCA1, BRCA2, BRIP1, CDK12, CHEK2, FANCA, PALB2, RAD51B, and RAD54L.

The present disclosure relates to a method of improving the median radiographic progression-free survival (rPFS) in a patient with metastatic castration-sensitive prostate cancer (mCSPC), who is positive for germline and/or somatic homologous recombination repair (HRR) gene alterations; said method comprising administering to said patient a daily dose of 200 mg of niraparib, 1000 mg of abiraterone acetate, and 5 mg of prednisone or prednisolone; wherein the HRR gene alterations are selected from the genes BRCA1, BRCA2, BRIP1, CDK12, CHEK2, FANCA, PALB2, RAD51B, and RAD54L; wherein said median rPFS is improved as indicated by a hazard ratio of 0.63 (95% CI: 0.49,0.80, p=0.0001) compared to a treatment with 1000 mg of abiraterone acetate and 5 mg of prednisone or prednisolone.

The present disclosure relates to a method of improving the median radiographic progression-free survival (rPFS) in a patient with metastatic castration-sensitive prostate cancer (mCSPC), who is positive for germline and/or somatic HRR gene alterations; said method comprising administering to said patient a daily dose of 200 mg of niraparib, 1000 mg of abiraterone acetate, and 5 mg of prednisone or prednisolone; wherein the HRR gene alterations are selected from the genes BRCA1, BRCA2, BRIP1, PALB2, RAD51B, and RAD54L; wherein said median rPFS is improved as indicated by a hazard ratio of 0.57 (95% CI: 0.42,0.77, p=0.0003) compared to a treatment with 1000 mg of abiraterone acetate and 5 mg of prednisone or prednisolone.

The present disclosure relates to a method of improving the median radiographic progression-free survival (rPFS) in a patient with metastatic castration-sensitive prostate cancer (mCSPC), who is positive for germline and/or somatic HRR gene alterations; said method comprising administering to said patient a daily dose of 200 mg of niraparib, 1000 mg of abiraterone acetate, and 5 mg of prednisone or prednisolone; wherein the HRR gene alterations are selected from the genes BRCA1 and BRCA2; wherein said median rPFS is improved as indicated by a hazard ratio of 0.52 (95% CI: 0.37,0.72, p<0.0001) compared to a treatment with 1000 mg of abiraterone acetate and 5 mg of prednisone or prednisolone.

The present disclosure relates to a method of improving the median time to symptomatic progression (TSP) in a patient with metastatic castration-sensitive prostate cancer (mCSPC), who is positive for germline and/or somatic HRR gene alterations; said method comprising administering to said patient a daily dose of 200 mg of niraparib, 1000 mg of abiraterone acetate, and 5 mg of prednisone or prednisolone; wherein the HRR gene alterations are selected from the genes BRCA1, BRCA2, BRIP1, CDK12, CHEK2, FANCA, PALB2, RAD51B, and RAD54L; wherein said median TSP is improved as indicated by a hazard ratio of 0.50 (95% CI: 0.36,0.69, p<0.0001) compared to a treatment with 1000 mg of abiraterone acetate and 5 mg of prednisone or prednisolone.

The present disclosure relates to a method of improving the median time to symptomatic progression (TSP) in a patient with metastatic castration-sensitive prostate cancer (mCSPC), who is positive for germline and/or somatic HRR gene alterations; said method comprising administering to said patient a daily dose of 200 mg of niraparib, 1000 mg of abiraterone acetate, and 5 mg of prednisone or prednisolone; wherein the HRR gene alterations are selected from the genes BRCA1, BRCA2, BRIP1, PALB2, RAD51B, and RAD54L; wherein said median TSP is improved as indicated by a hazard ratio of 0.49 (95% CI: 0.33,0.74, p=0.0004) compared to a treatment with 1000 mg of abiraterone acetate and 5 mg of prednisone or prednisolone.

The present disclosure relates to a method of improving the median time to symptomatic progression (TSP) in a patient with metastatic castration-sensitive prostate cancer (mCSPC), who is positive for germline and/or somatic HRR gene alterations; said method comprising administering to said patient a daily dose of 200 mg of niraparib, 1000 mg of abiraterone acetate, and 5 mg of prednisone or prednisolone; wherein the HRR gene alterations are selected from the genes BRCA1 and BRCA2; wherein said median TSP is improved as indicated by a hazard ratio of 0.44 (95% CI: 0.29,0.68, p=0.0001) compared to a treatment with 1000 mg of abiraterone acetate and 5 mg of prednisone or prednisolone.

The present disclosure relates to a method of improving the median overall survival (OS) in a patient with metastatic castration-sensitive prostate cancer (mCSPC), who is positive for germline and/or somatic HRR gene alterations; said method comprising administering to said patient a daily dose of 200 mg of niraparib, 1000 mg of abiraterone acetate, and 5 mg of prednisone or prednisolone; wherein the HRR gene alterations are selected from the genes BRCA1, BRCA2, BRIP1, CDK12, CHEK2, FANCA, PALB2, RAD51B, and RAD54L; wherein said median OS is improved as indicated by a hazard ratio of 0.79 (95% CI: 0.59,1.04, p=0.0951) compared to a treatment with 1000 mg of abiraterone acetate and 5 mg of prednisone or prednisolone.

The present disclosure relates to a method of improving the median overall survival (OS) in a patient with metastatic castration-sensitive prostate cancer (mCSPC), who is positive for germline and/or somatic HRR gene alterations; said method comprising administering to said patient a daily dose of 200 mg of niraparib, 1000 mg of abiraterone acetate, and 5 mg of prednisone or prednisolone; wherein the HRR gene alterations are selected from the genes BRCA1, BRCA2, BRIP1, PALB2, RAD51B, and RAD54L; wherein said median OS is improved as indicated by a hazard ratio of 0.81 (95% CI: 0.57,1.16, p=0.2485) compared to a treatment with 1000 mg of abiraterone acetate and 5 mg of prednisone or prednisolone.

The present disclosure relates to a method of improving the median overall survival (OS) in a patient with metastatic castration-sensitive prostate cancer (mCSPC), who is positive for germline and/or somatic HRR gene alterations; said method comprising administering to said patient a daily dose of 200 mg of niraparib, 1000 mg of abiraterone acetate, and 5 mg of prednisone or prednisolone; wherein the HRR gene alterations are selected from the genes BRCA1 and BRCA2; wherein said median OS is improved as indicated by a hazard ratio of 0.75 (95% CI: 0.51,1.11, p=0.1454) compared to a treatment with 1000 mg of abiraterone acetate and 5 mg of prednisone or prednisolone.

The present disclosure relates to a method of improving the time to subsequent therapy (TST) in a patient with metastatic castration-sensitive prostate cancer (mCSPC), who is positive for germline and/or somatic HRR gene alterations; said method comprising administering to said patient a daily dose of 200 mg of niraparib, 1000 mg of abiraterone acetate, and 5 mg of prednisone or prednisolone; wherein the HRR gene alterations are selected from the genes BRCA1, BRCA2, BRIP1, CDK12, CHEK2, FANCA, PALB2, RAD51B, and RAD54L; wherein said TST is improved as indicated by a hazard ratio of 0.54 (95% CI: 0.41,0.70, p<0.0001) compared to a treatment with 1000 mg of abiraterone acetate and 5 mg of prednisone or prednisolone.

The present disclosure relates to a method of improving the time to subsequent therapy (TST) in a patient with metastatic castration-sensitive prostate cancer (mCSPC), who is positive for germline and/or somatic HRR gene alterations; said method comprising administering to said patient a daily dose of 200 mg of niraparib, 1000 mg of abiraterone acetate, and 5 mg of prednisone or prednisolone; wherein the HRR gene alterations are selected from the genes BRCA1, BRCA2, BRIP1, PALB2, RAD51B, and RAD54L; wherein said TST is improved as indicated by a hazard ratio of 0.50 (95% CI: 0.36,0.69, p<0.0001) compared to a treatment with 1000 mg of abiraterone acetate and 5 mg of prednisone or prednisolone.

The present disclosure relates to a method of improving the time to subsequent therapy (TST) in a patient with metastatic castration-sensitive prostate cancer (mCSPC), who is positive for germline and/or somatic HRR gene alterations; said method comprising administering to said patient a daily dose of 200 mg of niraparib, 1000 mg of abiraterone acetate, and 5 mg of prednisone or prednisolone; wherein the HRR gene alterations are selected from the genes BRCA1 and BRCA2; wherein said TST is improved as indicated by a hazard ratio of 0.47 (95% CI: 0.33,0.66, p<0.0001) compared to a treatment with 1000 mg of abiraterone acetate and 5 mg of prednisone or prednisolone.

The present disclosure relates to a method of treating a patient with metastatic castration-sensitive prostate cancer (mCSPC) or metastatic hormone-sensitive prostate cancer (mHSPC), who is positive for germline and/or somatic HRR gene alterations; said method comprising administering to said patient a daily dose of 200 mg of niraparib, 1000 mg of abiraterone acetate, and 5 mg of prednisone or prednisolone, in combination with androgen deprivation therapy (ADT); wherein the HRR gene alterations are selected from the genes BRCA1 and BRCA2.

In one embodiment, the patient has an Eastern Cooperative Oncology Group Performance Status (ECOG PS) Grade <2.

In one embodiment, the patient has received and continues receiving androgen deprivation therapy (ADT).

ADT may be selected from leuprolide, goserelin, triptorelin, buserelin, histrelin, abarelix degarelix, relugolix, cyproterone acetate, flutamide, nilutamide, bicalutamide, enzalutamide, abiraterone acetate, seviteronel, apalutamide, darolutamide, and galeterone.

In one embodiment, the patient has received prior docetaxel treatment.

In one embodiment, the patient has an absolute neutrophil count ≥1.5×10⁹/L.

In one embodiment, the patient has hemoglobin ≥9.0 g/dL.

In one embodiment, the patient has platelet count ≥100×10⁹/L.

In one embodiment, the patient has creatinine <2× upper limit of normal (ULN).

In one embodiment, the patient has serum potassium ≥3.5 mmol/L.

In one embodiment, the patient has serum total bilirubin≤1.5×ULN or direct bilirubin≤1×ULN.

In one embodiment, the patient has AST or ALT≤3×ULN.

In one embodiment, the patient has not received prior treatment with a PARP inhibitor. Examples of PARP inhibitors include niraparib, olaparib, rucaparib, talazoparib, saruparib, veliparib.

In one embodiment, the patient has not received prior androgen receptor-targeted therapy, immunotherapy, or radiopharmaceutical agents for prostate cancer.

The androgen receptor-targeted therapy is selected from apalutamide, enzalutamide, darolutamide, nilutamide, flutamide, and bicalutamide.

In one embodiment, niraparib is administered as niraparib tosylate monohydrate.

In one embodiment, niraparib and abiraterone acetate are administered as a drug product or fixed-dose combination (FDC).

In one embodiment, niraparib and abiraterone acetate are administered as a free-dose combination (FrDC).

The present disclosure further relates to niraparib and abiraterone acetate for use in a method according to any one of the previous embodiments.

The present disclosure further relates to niraparib and abiraterone acetate for use in a method in combination with prednisone, according to any one of the previous embodiments.

The present disclosure further relates to niraparib and abiraterone acetate for use in a method in combination with prednisone and ADT, according to any one of the previous embodiments.

The present disclosure further relates to the use of niraparib and abiraterone acetate for the manufacture of a medicament for a method of improvement of a clinical endpoint in a patient with mCSPC as defined in any one of the previous embodiments.

In any of the embodiments presented herein regarding the medical uses of the drug product or methods of treatment with the drug product, the patient has previously received androgen deprivation therapy (ADT) or has undergone bilateral orchiectomy.

In any of the embodiments presented herein regarding the medical uses of the drug product or methods of treatment with the drug product, the patient has previously received gonadotropin-releasing hormone (GnRH) analogue therapy or has undergone bilateral orchiectomy.

The patient preferably continues receiving ADT, if not surgically castrated.

The patient preferably continues receiving GnRH analogue therapy, if not surgically castrated.

In any of the embodiments presented herein regarding the medical uses of the drug product or methods of treatment with the drug product, the patient has previously received chemotherapy selected from taxane chemotherapy, optionally docetaxel or cabazitaxel.

In any of the embodiments presented herein regarding the medical uses of the drug product or methods of treatment with the drug product, niraparib is in a salt form selected from tosylate monohydrate, sulfate, benzenesulfate, fumarate, succinate, camphorate, mandelate, camsylate, lauryl sulfate, or a mixture of tosylate monohydrate and lauryl sulfate.

In any of the embodiments presented herein regarding the medical uses of the drug product or methods of treatment with the drug product, the drug product—comprising the two-drug combination of 100 mg niraparib and 500 mg abiraterone acetate—is in the form of a film-coated tablet consisting of: i) a tablet core with the following excipients: Colloidal anhydrous silica, Crospovidone, Hypromellose, Lactose monohydrate, Magnesium stearate, Silicified microcrystalline cellulose, Sodium lauryl sulfate; and ii) a film-coating with the following excipients: Iron oxide red (E172), Iron oxide yellow (E172), Sodium lauryl sulphate, Glycerol monocaprylocaprate, Polyvinyl alcohol, Talc, and Titanium dioxide (E171).

In a preferred embodiment, the tablet has the following composition:

| | |
|---|---|
| Tablet components | Quantity (mg) |

Granule composition:
Binder Solution:

| | |
|---|---|
| HPMC 2910 15 mPa · s | 24.00 |
| Sodium Lauryl Sulfate | 5.60 |
| Purified Water^{a} | <800.00^{a}> |

Intragranular Phase:

| | |
|---|---|
| Abiraterone acetate | 500.00 |
| Niraparib tosylate monohydrate^{b} | 159.40^{b}. |
| Lactose monohydrate | 253.20 |
| Crospovidone | 32.00 |

Extragranular Phase:

| | |
|---|---|
| Silicified Microcrystalline Cellulose | 461.80 |
| Crospovidone | 80.00 |
| Sodium Lauryl Sulfate | 56.00 |
| Colloidal Anhydrous Silica | 12.00 |
| Magnesium Stearate | 16.00 |
| Tablet weight: | 1600.00 |

^{a}wherein said Purified Water is removed during processing;
^{b}wherein the salt factor is 1.594; 159.40 mg niraparib tosylate is equivalent to 100.00 mg dose of niraparib; and wherein the tablet is film-coated with about 64 mg of the coating powder Opadry ® AMB II 88A170010 Beige and 256 mg of purified water, wherein the latter purified water is removed during processing.

In any of the embodiments presented herein regarding the medical uses of the drug product or methods of treatment with the drug product, the drug product—comprising the two-drug combination of 100 mg niraparib and 500 mg abiraterone acetate—is in the form of a capsule.

The capsule comprises: i) the two-drug combination of 100 mg niraparib and 500 mg abiraterone acetate, and ii) a pharmaceutically acceptable carrier comprising Crospovidone, Hypromellose, Sodium lauryl sulfate, Lactose monohydrate, and Magnesium stearate.

Preferably, niraparib and abiraterone acetate as a FrDC, or two film-coated tablets or two capsules are administered, daily, at least two hours after eating and food must not be eaten for at least one hour after administration.

In any of the embodiments presented herein regarding the medical uses of the drug product or methods of treatment with the drug product, the germline and/or somatic HRR gene alteration(s) are determined by using a validated test method.

Examples of Commercially Available Germline Genetic Tests and Somatic Tests for Prostate Cancer are provided herein below:

TABLE 1
A) Germline testing

| | | | Prostate-specific panels or hereditary |
|---|---|---|---|
| Test | Company | Sample type | cancer panels |
| ProstateNext ® | Ambry Genetics | Blood /saliva | Multi-gene prostate panel: 14 genes (ATM, |
| | (Aliso Viejo, CA, | | BRCA1, BRCA2, CHEK2, EPCAM, |
| | USA) | | HOXB13, MLH1, MSH2, MSH6, NBN, |
| | | | PALB2, PMS2, RAD51D, and TP53) |
| Color Hereditary | Color Genomics | Blood | 30-gene panel including BRCA1, BRCA2, |
| Cancer Test | (Burlingame, CA, | | MLH1, MSH2, MSH6, PMS2, EPCAM, |
| | USA) | | CHEK2, TP53, ATM, and NBN |
| Invitae Prostate | Invitae Corporation | Blood /saliva | Multi-gene prostate panel: 12 genes (ATM, |
| Cancer Panel | (San Francisco, CA, | | BRCA1, BRCA2, CHEK2, EPCAM, |
| | USA) | | HOXB13, MLH1, MSH2, MSH6, NBN, |
| | | | PMS2, and TP53) |
| Prostate Gene | GeneHealth | Blood | Multi-gene prostate panel: 11 genes |
| | (Cambridge, UK) | | (BRCA1, BRCA2, HOXB13, MLH1, MSH2, |
| | | | MSH6, PMS2, EPCAM, ATM, CHEK2) |
| Myriad myRisk ® | Myriad Genetics, | Blood | 35-gene panel including BRCA1, BRCA2, |
| Hereditary Cancer | Inc. (Salt Lake City, | | MLH1, MSH2, MSH6, PMS2, EPCAM, |
| Test | UT, USA) | | TP53, NBN, HOXB13, ATM, CHEK2 |

B) Somatic testing for advanced/metastatic disease

| Test | Company | Sample type | Prostate-specific panels |
|---|---|---|---|
| FoundationOne | Foundation Medicine | Tumor | 14 HRR pathway genes |
| CDx | Inc. (Cambridge, MA) | biopsy | within 324 gene panel |
| FoundationOne | Foundation Medicine | Blood | 14 prostate specific genes |
| Liquid CDx | Inc. (Cambridge, MA) | | within 324 gene panel |
| Caris Molecular | Caris Life Sciences | Tumor | NA |
| Intelligence | (Irving, TX) | biopsy | |
| Guardant360 | Guardant Health Inc. | Blood/ | 70+ genes |
| | (Redwood City, CA) | Circulating | |
| | | tumor DNA | |

The present disclosure further relates to any of the methods presented herein, said method further comprising selling such drug product, wherein a drug product label for a reference listed drug for such drug product includes instructions for treating mCSPC.

In one embodiment, the drug product label comprises rPFS, OS, TSP, TST, TCC, TPP, or ORR data.

The present disclosure further relates to selling such drug product, wherein a drug product label for a reference listed drug for such approval drug product comprises rPFS, OS, TSP, TST, TCC, TPP, or ORR data.

### Niraparib

Niraparib is an orally available highly selective poly (adenosine diphosphate [ADP]-ribose) polymerase (PARP) inhibitor, with activity against PARP-1 and PARP-2 deoxyribonucleic acid (DNA)-repair polymerases. The preparation of niraparib is described in U.S. Pat. Nos. 8,071,623 and 8,436,185, both of which are incorporated herein by reference.

Niraparib is currently marketed under the ZEJULA® brand as a capsule formulation that contains 159.4 mg niraparib tosylate monohydrate (equivalent (eq.) to 100 mg niraparib free base) as the active ingredient.

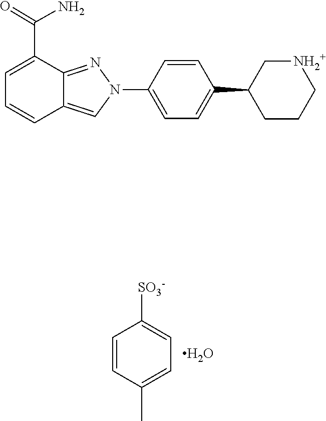

As used herein, the term “niraparib” means any of the free base compound (2-[4-[(3S)-piperidin-3-yl]phenyl]-2H-indazole-7-carboxamide), a salt form, including pharmaceutically acceptable salts, of 2-[4-[(3S)-piperidin-3-yl]phenyl]-2H-indazole-7-carboxamide (e.g., 4-methylbenzenesulfonic acid; 2-[4-[(3S)-piperidin-3-yl]phenyl]-2H-indazole-7-carboxamide), and/or a solvated form, including a hydrated form, thereof (e.g., 2-[4-[(3S)-piperidin-3-yl]phenyl]-2H-indazole-7-carboxamide tosylate monohydrate). Such forms may be individually referred to as “niraparib free base”, “niraparib tosylate” and “niraparib tosylate monohydrate”, respectively.

The term “niraparib eq.” or “niraparib equivalent” refers to the free base dose amount of niraparib.

### Abiraterone Acetate

Abiraterone acetate is a compound of formula:

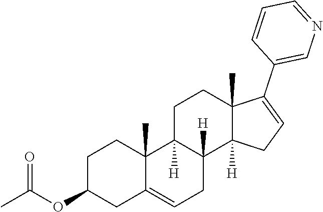

and is a prodrug of abiraterone, which is a potent selective, orally active inhibitor of the key enzyme in testosterone synthesis, 17α-hydroxylase-C17,20-lyase, also known as steroid 17a-monooxygenase inhibitor or Human Cytochrome P45017α. Suppression of testosterone synthesis has been demonstrated with abiraterone acetate in patients with prostate cancer. The compound was disclosed in WO 93/20097 (A1).

Abiraterone acetate plus prednisone is approved for use in patients with metastatic castration-resistant prostate cancer (mCRPC) or metastatic hormone-sensitive prostate cancer (mHSPC). Abiraterone acetate tablets are currently on the market as 250 or 500 mg oral tablets.

### Methods of Treatment and Medical Uses

The methods for treating a prostate cancer, or the medical uses of the present disclosure comprise, consist of and/or consist essentially of, administering to a patient in need thereof a clinically effective amount of niraparib, a clinically effective amount of abiraterone acetate, and optionally a clinically effective amount of another drug product, for example a glucocorticoid, for example prednisone or prednisolone.

The methods for treating a prostate cancer, or the medical uses of the present disclosure comprise, consist of and/or consist essentially of, administering to a patient in need thereof niraparib and abiraterone acetate, as a FrDC or formulated into a single oral dosage form and administered in a clinically effective amount. The methods for treating a prostate cancer, or the medical uses of the present disclosure comprise, consist of and/or consist essentially of, administering to a patient in need thereof the afore-mentioned combination, plus a glucocorticoid, for example prednisone or prednisolone in a clinically effective amount.

Also disclosed are dosage regimens of the oral dosage forms disclosed herein, said dosage regimens comprising, consisting of and/or consisting essentially of, administering the two-drug combination or FrDC, the dual combination, or FDC of niraparib and abiraterone acetate, and optionally plus a glucocorticoid, for example prednisone or prednisolone, in a total amount that is clinically effective for the treatment of prostate cancer in a human.

The present disclosure also discloses kits comprising, consisting of, and/or consisting essentially of, a dual combination of niraparib and abiraterone acetate, a triple combination of niraparib, abiraterone acetate, and prednisone or prednisolone, or a FDC comprising niraparib and abiraterone acetate, and an instruction print for administering said combination to a human patient having a prostate cancer.

The kits may comprise, consist, and/or consist essentially of, the dual combination, the triple combination, the FDC comprising niraparib and abiraterone acetate, a separate composition that comprises a glucocorticoid, for example prednisone or prednisolone; and an instruction print for administering the combination to a human patient having a prostate cancer.

Where a particular reference is made “prednisone” in the present disclosure, one of ordinary skill will recognize that prednisone may be substituted with a different glucocorticoid, such as prednisolone, hydrocortisone, methyl prednisolone, or dexamethasone. The person skilled in the art will know how to exchange prednisone with these other drugs and adjust their dosage, if necessary.

Particular suitable glucocorticoids include but are not limited to, (1) dexamethasone (e.g., Decadron, oral; Decadron-LA injection, etc.), (2) prednisolone (e.g., Delta-CORTEF®, prednisolone acetate (ECONOPRED®), prednisolone sodium phosphate (HYDELTRASOL®), prednisolone tebutate (HYDELTRA-TBA®, etc.)), (3) prednisone (DELTASONE®, etc.), or (4) methylprednisolone (e.g., MEDROL®), and combinations thereof. See, e g., Goodman & Gilman's The Pharmacological Basis of Therapeutics, 10th edition 2001.

The combinations and formulations described herein are used in methods of treating prostate cancer patients with homologous recombination deficiency (HRD) positive biomarker status. HRD is also referred to as homologous recombination repair (HRR) gene defects or alterations and can result from DNA repair gene defects (DRD). HRD or HRR gene alterations encompass DRD and also those gene mutations or alterations outside of the DNA-repair pathway. Said HRD—or HRR gene defects or alterations—positive status may be detected by evaluating somatic or germline alterations, or by evaluating genome-wide loss of heterozygosity (LOH), or homozygous deleterious changes in DNA repair genes. HRD—or HRR gene defects or alterations—positive status is also a synonym for PARP biomarker positive status.

The positive biomarker status may be HRR-positive status. HRR positive status may be defined as having monoallelic or biallelic, germline and/or somatic alterations in one or more DNA repair genes, including without being limited to, alterations in BRCA2 (Breast Cancer gene 2), BRCA1 (Breast Cancer gene 1), ATM (ataxia-telangiectasia mutated), BRIP1 (BRCA1 Interacting Protein C-terminal Helicase 1 gene), CHEK2 (Checkpoint Kinase 2 gene), FANCA (Fanconi Anemia Complementation Group A gene), PALB2 (Partner and Localizer of BRCA2 gene), CDK12 (Cyclin Dependent Kinase 12), RAD51B (RAD51 Paralog B), or RAD54L (RAD54 Like).

BRCA alterations refer to alterations in the BRCA1 and BRCA2 genes.

HRR Effectors alterations refer to alteration in genes: BRCA1, BRCA2, BRIP1, PALB2, RAD51B, RAD54L.

All HRR subjects or population refers to patients having alterations in genes: BRCA1, BRCA2, BRIP1, CDK12, CHEK2, FANCA, PALB2, RAD51B, and RAD54L.

The germline and/or somatic HRR gene alteration is determined by using a validated test method. HRR status may be preferably evaluated by either a plasma-(Resolution Bioscience) or tissue-based test (Foundation Medicine), particularly by detecting circulating plasma DNA or circulating tumor cells. A list of tests for determining germline and/or somatic HRR gene alterations is provided herein above.

Gene expression profile analysis and protein biomarkers may also be used to risk-stratify patients with prostate cancer to guide treatment decisions. Commercially available tests include Prolaris® (Myriad Genetics, Salt Lake City, UT); Oncotype Dx® Prostate Cancer Assay (Genomic Health, Redwood City, CA); ProMark™ Protein Biomarker Test/ProMark™ Risk Score (Metamark Genetics, Cambridge, MA); FoundationOne® CDx (Foundation Medicine, Cambridge, MA); FoundationOne® Liquid CDx (Foundation Medicine, Cambridge, MA); Caris Molecular Intelligence (Caris Life Sciences, Irving, TX); Guardant360 (Guardant Health Inc., Redwood City, CA); ProstateNext® (Ambry Genetics, Aliso Viejo, CA); Color Hereditary Cancer Test (Color Genomics, Burlingame, CA); Invitae Prostate Cancer Panel (Invitae Corp., San Francisco, CA); Prostate Gene (GeneHealth, Cambridge, UK); Myriad myRisk® Hereditary Cancer Test (Myriad Genetics Inc., Salt Lake City, UT) and Decipher® Prostate Cancer Test (GenomeDx Biosciences, San Diego, CA), this latter based on the expression pattern of 22 RNA markers in biopsy or radical prostatectomy specimens. Prolaris®, OncotypeDx®, and Decipher® are tissue-based gene expression tests.

The FrDC, FDC, or formulations described herein may be used in methods of treating prostate cancer in patients with detectable circulating tumor cells (CTC), circulating DNA, or reduction of plasma DNA. The formulations described herein may be used in methods of treating metastatic prostate cancer in patients with detectable CTCs and/or measurable and non-measurable bony disease or lesions. CTC clearance in patients with metastatic prostate cancer may be established when detecting ≥5 cells per 7.5 mL blood at baseline, detecting <5 cells per 7.5 mL blood at nadir, further confirmed by a second consecutive value obtained 4 or more weeks later.

The subject may be surgically castrated or chemically castrated.

The patient may have undergone one or more other types of treatment or therapies for prostate cancer prior to the first dose of the two-drug combination, FrDC, dual combination, or FDC of niraparib and abiraterone acetate. For example, the patient may have undergone taxane-based chemotherapy prior to administering the combination of niraparib and abiraterone acetate. Additionally, or alternatively, the patient may have undergone at least one line of androgen receptor-targeted therapy, such as nilutamide, flutamide, bicalutamide, enzalutamide, apalutamide, or darolutamide, prior to administering the combination of niraparib and abiraterone acetate. In an aspect, the patient does not respond initially or becomes refractory to previous treatments, prior to administering the combination of niraparib and abiraterone acetate. Optionally the glucocorticoid, for example prednisone or prednisolone, can also be administered in addition to the combination of niraparib and abiraterone acetate.

In one embodiment, two Akeega® tablets comprising the two-drug combination, the dual combination, or FDC of niraparib and abiraterone acetate may be administered once daily, at least 1 hour before a meal or at least two hours after a meal. In an embodiment, two Akeega® tablets comprising the two-drug combination, the dual combination, or FDC of niraparib and abiraterone acetate may be administered once daily, with water, on an empty stomach at least 1 hour before a meal or at least two hours after meal.

Alternatively, one or more tablets or capsules comprising niraparib, and one or more tablets or capsules comprising abiraterone acetate are administered once daily.

In an embodiment a glucocorticoid is administered once or twice daily. In an embodiment, prednisone or prednisolone tablets or capsules are administered once or twice daily.

In an embodiment, 1 or 2 tablets or capsules comprising the two-drug combination, the dual combination, or FDC of niraparib and abiraterone acetate are administered once daily and 1 tablet or capsule of a glucocorticoid, for example prednisone is administered once or twice daily.

When the two-drug combination, the dual combination, or FDC of niraparib and abiraterone acetate is administered to a patient, the selected dosage level for each drug will depend on a variety of factors including, but not limited to, the activity of the particular compound, the severity of the individual's symptoms, the route of administration, the time of administration, the rate of excretion of the compound, the duration of the treatment, other drugs, compounds, and/or materials used in combination, and the age, sex, weight, condition, general health, and prior medical history of the patient. The amount of niraparib, the amount of abiraterone acetate, and optionally the amount of prednisone or prednisolone, will ultimately be at the discretion of the physician, although generally the dosage will be to achieve local concentrations at the site of action which achieve the desired effect without causing substantial harmful or deleterious side-effects.

The two-drug combination, the dual combination, or FDC may comprise, for example, about 33 to about 350 mg of the niraparib, about 100 to about 1500 mg of the abiraterone acetate. Preferably the FDC comprises 100 mg of niraparib and 500 mg of abiraterone acetate. Alternatively, the FDC comprises 50 mg of niraparib and 500 mg of abiraterone acetate.

Examples of anti-cancer agent include docetaxel, mitoxantrone, cabazitaxel, cisplatin, carboplatin, oxaliplatin, and etoposide.

Examples of immunotherapeutic agents include for example pembrolizumab, sipuleucel-T. Examples of bone-targeted therapies include for example denosumab, zoledronic acid, alendronate, radium-223, strontium-89, samarium-153.

Example of gonadotropin releasing hormone agonists (GnRHa) include, without being limited to, triptorelin, nafarelin, goserelin, leuprorelin or leuprolide, histrelin, gonadorelin, and buserelin).

Example of hormone therapies include for example nilutamide, flutamide, bicalutamide, goserelin, histrelin, leuprolide, triptorelin, degarelix, enzalutamide, apalutamide, darolutamide, diethylstilbestrol, estrogens.

An example of radiation therapy is external beam radiation therapy (EBRT).

Examples of alternative energy sources include high-intensity focused ultrasound (HIFU), cryosurgery, and laser treatments.

Also disclosed herein are kits including a FrDC or FDC of niraparib and abiraterone acetate, and optionally a separate composition that comprises prednisone or prednisolone, and instructions for administering the FrDC or FDC to a human patient having prostate cancer. The instructions may provide instructions for administering the respective FrDC or FDC once daily. For example, the instruction print may provide instructions for administering the FrDC or FDC comprising niraparib and abiraterone acetate to a human patient having prostate cancer on a once daily basis, and optionally for administering the composition comprising prednisone or prednisolone to the human patient on a single or twice daily basis.

The present disclosure further relates to a method for determining the bioequivalence of a test FrDC or FDC formulation of niraparib and abiraterone acetate, relative to an oral dosage form of the present disclosure, said method comprising i) measuring a bioequivalence parameter of the test FrDC or FDC formulation and optionally measuring a bioequivalence parameter of the FrDC or FDC of the present disclosure, and ii) comparing the bioequivalence parameter of the test FrDC or FDC formulation to the corresponding bioequivalence parameter of the FrDC or FDC of the present disclosure.

In an aspect, the bioequivalence parameter is selected from AUC₍₀₋₁₎, AUC_{(0-∞)}, residual area, Cₘₐₓ and tₘₐₓ, AUC₍₀₋₇₂ₕ₎, terminal rate constant (λ_{z}), t_{1/2}, AUC_{(0-τ)}, C_{max,ss}, t_{max,ss}, Ae₍₀₋ₜ₎, and Rₘₐₓ, which bioequivalence parameters are well known to the person skilled in the arts of bioequivalence and pharmacokinetics.

### Methods of Sale

In another aspect, described herein are methods of selling the FrDC or FDC of the disclosure comprising, consisting of, or consisting essentially of placing the FrDC or FDC into the stream of commerce wherein said FrDC or FDC is accompanied with a package insert that contains instructions for safely and effectively treating prostate cancer using the FrDC or FDC.

In further aspects, described herein are methods of selling a FrDC or a pharmaceutical composition containing the two-drug combination as a FDC, comprising, consisting of, or consisting essentially of placing such FrDC or FDC pharmaceutical composition into the stream of commerce wherein such FrDC or FDC pharmaceutical composition is accompanied with a package insert that contains instructions for safely and effectively treating prostate cancer using said FrDC or FDC.

In further aspects, described herein are methods of selling the two-drug combination as a free dose combination (FrDC), comprising, consisting of, or consisting essentially of placing such niraparib and abiraterone acetate separately into the stream of commerce wherein such niraparib and abiraterone acetate are each independently accompanied with a package insert that contains instructions for safely and effectively treating prostate cancer using the two-drug combination.

In still further aspects, described herein are methods of offering for sale the two-drug combination comprising, consisting of, or consisting essentially of offering to place the two-drug combination into the stream of commerce wherein said two-drug combination is accompanied with a package insert that contains instructions for safely and effectively treating prostate cancer using the two-drug combination.

The present disclosure is further defined in the following examples which include unexpected and advantageous results. It should be understood that these examples, while indicating preferred embodiments of the disclosure, are given by way of illustration only, and should not be construed as limiting the appended claims. From the above discussion and these examples, one skilled in the art can ascertain the essential characteristics of this disclosure, and without departing from the spirit and scope thereof, can make various changes and modifications of the disclosure to adapt it to various usages and conditions.

### EXAMPLES

### Example 1—A Phase 3 Randomized, Placebo-controlled, Double-blind Study of Niraparib in Combination with Abiraterone Acetate and Prednisone Versus Abiraterone Acetate and Prednisone for the Treatment of Participants with Deleterious Germline or Somatic Homologous Recombination Repair (HRR) Gene-Mutated Metastatic Castration-Sensitive Prostate Cancer (mCSPC), AMPLITUDE

The objective of this study was: (1) to determine if niraparib and abiraterone acetate (AA), plus prednisone compared with AA plus prednisone in participants with deleterious germline or somatic HRR gene-mutated mCSPC provides superior efficacy in improving radiographic progression-free survival (rPFS); (2) to assess the clinical benefit of niraparib and AA, plus prednisone compared with AA plus prednisone in participants with deleterious germline or somatic HRR gene-mutated mCSPC; and (3) to characterize the safety profile of niraparib and AA, plus prednisone compared with AA plus prednisone in participants with deleterious germline or somatic HRR gene-mutated mCSPC.

### Overall Design

Approximately 692 participants were randomly assigned in a 1:1 ratio to either niraparib 200 mg, and AA 1000 mg, plus prednisone 5 mg daily or AA 1000 mg plus prednisone 5 mg daily. All participants had to be receiving background androgen deprivation therapy (ADT; ie, gonadotropin-releasing hormone analogue (GnRHa) or surgical castration).

The study consisted of 4 phases: a Prescreening Phase for biomarker evaluation for eligibility only, a Screening Phase, a Treatment Phase, and a Follow-up Phase.

Efficacy, safety, pharmacokinetics (PK), and biomarkers were assessed according to the Schedule of Activities (SoA).

Treatment was continuous; however, a treatment cycle was defined as 28 days. Study medication ought to be continued until disease progression, unacceptable toxicity, death, withdrawal of consent, or termination of the study by the sponsor. Patients with radiographic progression could remain on therapy if still receiving clinical benefit.

### Efficacy Evaluations

The primary endpoint for this study was rPFS as assessed by the investigator which was evaluated using computed tomography or magnetic resonance imaging scans and whole-body bone scans (Technetium 99m). Evaluation of rPFS was also assessed by blinded independent central review.

Radiographic progression was evaluated as follows:
Progression of soft tissue lesions measured by CT or MRI as defined in RECIST 1.1.
Progression by bone lesions observed by bone scan based on PCWG3. Under these criteria, any bone progression had to be confirmed by a subsequent scan ≥6 weeks later. The Week 8 scan (first post-treatment scan, ie, Cycle 3 Day 1) was to be used as the reference to which all subsequent scans were compared to determine progression. Bone progression was defined as one of the following:
   1. Participant whose Week 8 scan was observed to have ≥2 new bone lesions would fall into one of the 2 categories below:
      a) Participant whose confirmatory scan (which is performed ≥6 weeks later) showed ≥2 new lesions compared with the Week 8 scan (ie, a total of ≥4 new lesions compared to baseline scan) were to be considered to have bone scan progression at Week 8.
      b) Participant whose confirmatory scan did not show ≥2 new lesions compared with the Week 8 scan were not to be considered to have bone scan progression. The Week 8 scan was to be considered as the reference scan to which subsequent scans were compared. The FIRST scan timepoint that showed ≥2 new lesions compared with the Week 8 scan were to be considered as the bone scan progression timepoint if these new lesions were confirmed by a subsequent scan ≥6 weeks later.
   2. For a participant whose Week 8 scan did not have ≥2 new bone lesions compared with the baseline scan, the FIRST scan timepoint that showed ≥2 new lesions compared with the Week 8 scan was to be considered as the bone scan progression timepoint if these new lesions were confirmed by a subsequent scan ≥6 weeks later.

Other efficacy evaluations included the following:
Survival status
Symptomatic progression
Subsequent systemic therapy for prostate cancer
Cancer-related radiation therapy or surgical procedures
Serum PSA
PROS
ECOG PS

### Biomarker Evaluations

Deleterious germline or somatic HRR gene alterations were evaluated using the sponsor's approved assays to determine molecular eligibility at prescreening from (a) tumor tissue (archival or recently collected), and (b) germline (eg, blood, saliva), and also (c) plasma (if test was available from the sponsor).

TABLE 2
Eligible HRR Gene Alterations

| Genes | Definition |
|---|---|
| BRCA1 | Breast Cancer gene 1 |
| BRCA2 | Breast Cancer gene 2 |
| BRIP1 | BRCA1 Interacting Protein C-terminal Helicase 1 gene |
| CDK12 | Cyclin Dependent Kinase 12 |
| CHEK2 | Checkpoint Kinase 2 gene |
| FANCA | Fanconi Anemia Complementation Group A gene |
| PALB2 | Partner and Localizer of BRCA2 gene |
| RAD51B | RAD51 paralog B |
| RAD54L | RAD54-Like |

### Safety Evaluations

Safety assessments were based on review of adverse events (AE) reports and the results of heart rate and blood pressure measurements, electrocardiograms (ECGs), physical examinations, and clinical safety laboratory tests at specified timepoints.

### Justification for Dose

The dose of niraparib in this study is 200 mg administered with AA 1000 mg in combination with prednisone 5 mg; all study medications were administered once daily.

### Study Population

Men >18 years of age (or the local legal age of consent) with deleterious germline or somatic HRR gene-mutated metastatic prostate cancer were eligible for this study. The inclusion and exclusion criteria for enrolling participants in this study are described below.

### Inclusion Criteria

Each potential participant had to satisfy all of the following criteria to be enrolled in the study:
1. ≥18 years of age (or the local legal age of consent).
2. Pathological diagnosis of prostate adenocarcinoma.
3. Metastatic disease documented by conventional imaging with CT or MRI (for soft tissue lesions) or 99mTc bone scan (for bone lesions). Participants with a single bone lesion on 99mTc bone scan with no other non-nodal metastatic disease had to have confirmation of bone metastasis by CT or MRI.
   a. Participants with lymph node-only disease were not eligible.
4. Had to have at least one of the deleterious germline or somatic HRR gene alterations listed in Table 2.
5. Eastern Cooperative Oncology Group Performance Status (ECOG PS) Grade ≤2.
6. Androgen deprivation therapy (either medical or surgical castration) had to have been started ≥14 days prior to randomization and participants had to be willing to continue ADT through the treatment phase. Participants who started a GnRH agonist ≤28 days prior to randomization were required to take a first-generation anti-androgen for ≥14 days prior to randomization. The anti-androgen had to be discontinued prior to randomization.
7. Participants who had received prior docetaxel treatment had to meet the following criteria:
   a. Received a maximum of 6 cycles of docetaxel therapy for mCSPC
   b. Received the last dose of docetaxel ≤3 months prior to randomization
   c. Maintained a response to docetaxel of stable disease or better, by investigator assessment of imaging and/or PSA, prior to randomization.
8. Other allowed prior therapy for mCSPC:
   a. Maximum of 1 course of radiation and 1 surgical intervention for symptomatic control of prostate cancer (eg, uncontrolled pain, impending spinal cord compression or obstructive symptoms). Participants with radiation or surgical interventions to all known sites of metastatic disease were excluded from trial participation. Radiation had to be completed prior to randomization.
   b. Up to a maximum of 6 months of ADT prior to randomization.
   c. Up to a maximum of 45 days of AAP prior to randomization.
   d. Up to a maximum of 2 weeks of ketoconazole for prostate cancer prior to randomization.
9. Allowed prior treatments for localized prostate cancer include radical prostatectomy (with or without lymph node dissection), radiation therapy, and other locally directed treatments to the prostate per institutional standards of care.
   a. Participants who received ADT or first-generation anti-androgens for the treatment of localized prostate cancer
      i. ADT: must have had ≤3 years total and must have completed ≥1 year prior to randomization
      ii. First-generation anti-androgen: must have had ≤3 years total and must have completed ≥1 year prior to randomization.
10. Clinical laboratory values at Screening:
   a. Absolute neutrophil count ≥1.5×10°/L
   b. Hemoglobin ≥9.0 g/dL, independent of transfusions for at least 28 days
   c. Platelet count ≥100×10⁹/L
   d. Creatinine ≤2× upper limit of normal (ULN)
   e. Serum potassium ≥3.5 mmol/L
   f. Serum total bilirubin ≤1.5× ULN or direct bilirubin ≤1× ULN (Note: In participants with Gilbert's syndrome, if total bilirubin is >1.5× ULN, measure direct and indirect bilirubin, and if direct bilirubin is ≤1.5× ULN, participant may be eligible)
   g. AST or ALT≤3× ULN
11. Able to swallow the study medication tablets whole.

### Exclusion Criteria

Any potential participant who met any of the following criteria were excluded from participating in the study:
1. Pathological finding consistent with small cell or neuroendocrine carcinoma of the prostate.
2. Prior treatment with a PARP inhibitor.
3. Prior AR-targeted therapy (eg, apalutamide, enzalutamide, darolutamide), immunotherapy, or radiopharmaceutical agents for prostate cancer with the Exception: allowed prior therapies are noted in inclusion criteria 8.
4. History of adrenal dysfunction
5. Long-term use of systemically administered corticosteroids (>5 mg of prednisone or the equivalent) during the study was not allowed. Short-term use (≤4 weeks, including taper) and locally administered steroids (eg, inhaled, topical, ophthalmic, and intra-articular) were allowed, if clinically indicated.
6. Active malignancies (ie, progressing or requiring treatment change in the last 24 months) other than the disease being treated under study. The only allowed exceptions were:
   a. non-muscle invasive bladder cancer;
   b. skin cancer (non-melanoma or melanoma) treated within the last 24 months that is considered completely cured;
   c. breast cancer-adequately treated lobular carcinoma in situ or ductal carcinoma in situ;
   d. malignancy that is considered cured with minimal risk of recurrence.
7. History or contemporaneous diagnosis of MDS/AML.
8. Contemporaneous evidence within 6 months prior to randomization of any of the following: severe/unstable angina, myocardial infarction, symptomatic congestive heart failure, clinically significant arterial or venous thromboembolic events (ie. pulmonary embolism), or clinically significant ventricular arrhythmias.
9. Presence of sustained uncontrolled hypertension (systolic blood pressure >160 mm Hg or diastolic blood pressure >100 mm Hg). Participants with a history of hypertension were allowed, provided that blood pressure was controlled to within these limits by an anti-hypertensive treatment.
10. Known allergies, hypersensitivity, or intolerance to the excipients of niraparib, AA, or niraparib/AA FDC.
11. Contemporaneous evidence of any medical condition that would have made prednisone use contraindicated.
12. Received an investigational intervention (including investigational vaccines) or used an invasive investigational medical device within 30 days before the planned first dose of study medication.
13. Participants who had had the following ≤28 days prior to randomization:
   a. A transfusion (platelets or red blood cells);
   b. Hematopoietic growth factors;
   c. Major surgery (sponsor should be consulted regarding what constitutes major surgery).
14. Human immunodeficiency virus positive participants with 1 or more of the following:
   a. Not receiving highly active antiretroviral therapy or on antiretroviral therapy for less than 4 weeks.
   b. Receiving antiretroviral therapy that might interfere with the study medication (consult the sponsor for review of medication prior to enrollment).
   c. A change in antiretroviral therapy within 6 months of the start of screening (except if, after consultation with the sponsor on exclusion criterion 14.b, a change was made to avoid a potential drug-drug interaction with the study medication).
   d. CD4 count <350 at screening.
   e. An acquired immunodeficiency syndrome-defining opportunistic infection within 6 months of the start of screening.
   f. Human immunodeficiency virus load ≥400 copies/mL.
15. Active or symptomatic viral hepatitis or chronic liver disease; encephalopathy, ascites or bleeding disorders secondary to hepatic dysfunction.
16. Moderate or severe hepatic impairment (Class B and C per Child-Pugh classification system.

### Dose Modification

Any dose/dosage adjustment had to be overseen by medically qualified study-site personnel (principal or sub-investigator unless an immediate safety risk appears to be present).

All dose interruptions and dose reductions (including missed doses) and the reason for the interruption/reduction were to be recorded. Management of toxicities had to be performed as detailed in Table 3.

Once the dose of study medication was reduced, any re-escalation had to be discussed in advance with the sponsor's medical monitor.

In this section, the terms niraparib and AA refer to FDC (regular or low strength), single-agents (niraparib and AA) or relevant placebos.

In general, dose interruptions/modifications were to be managed as follows:
The dose of prednisone could remain unchanged with dose modifications of niraparib and AA.
If niraparib was interrupted or permanently discontinued due to toxicity, then AA could be continued. If AA was interrupted or permanently discontinued due to toxicity, then niraparib could be continued. If AA was permanently discontinued, prednisone could also be discontinued (with a taper if clinically indicated).
Niraparib had to be discontinued for non-hematologic treatment-related Grade ≥3 toxicities lasting more than 28 days while the participant was administered niraparib 100 mg once daily.

General guidelines for dose modifications are provided in Table 3.

TABLE 3
General Dose Reduction Guidelines for Participants on the FDC Treatment Regimen

| | Dose of | | |
|---|---|---|---|
| Dose Required | Niraparib | Dose of AA | Instruction |
| Full dose | 200 mg | 1000 mg | Participants were to receive regular-strength FDC |
| | | | tablets (active or placebo) and AA tablets (active |
| | | | or placebo). |
| Reduced | 100 mg | 1000 mg | Participants were to receive low-strength FDC |
| niraparib and full | | | (active or placebo) and AA tablets (active or |
| dose AA | | | placebo). |
| Full dose | 200 mg | 500 mg | Participants were to receive a single-agent |
| niraparib and | | | combination of niraparib (active or placebo) and |
| reduced AA | | | AA. |
| Reduced both | 100 mg | 500 mg | Participants were to receive one regular-strength |
| niraparib and | | | FDC tablet (active or placebo) and two AA tablets |
| AA | | | (active or placebo). |
| AA only | | 500 or | Participants were to receive single-agent AA if |
| | | 1000 mg | niraparib (active or placebo) was interrupted or |
| | | | discontinued. |
| Niraparib only | 100 or | | Participants were to receive single-agent niraparib |
| | 200 mg | | (active or placebo) if AA was interrupted or |
| | | | discontinued. |

AA = abiraterone acetate;
FDC = fixed-dose combination

### Statistical Analyses

The Statistical Analysis Plan (SAP) was finalized prior to database lock.

All continuous variables were summarized using number of participant (n), mean, standard deviation (SD), median, minimum, and maximum. Discrete variables were summarized with number and percent. All efficacy endpoints were analyzed using the FAS population. The Kaplan Meier product limit method and a stratified Cox model were used to estimate the time-to-event variables and to obtain the HR along with the associated confidence intervals. Unless otherwise specified, stratified log-rank tests were used to test the treatment effect for time-to-event variables; response rate variables were evaluated using the chi-square statistic or the Fisher's exact test if the cell counts were small.

### Multiplicity Adjustment for Testing of Primary and Key Secondary Endpoints

The SAP provides details on the graphical testing framework that was used to strictly control the familywise type 1 error rate at 2-sided 0.05 level.

### Primary Endpoint

The primary endpoint was rPFS, as assessed by the investigator, and defined as the time from the date of randomization to the date of radiographic progression or death, whichever occured first.

### Secondary Endpoints

The secondary endpoints were defined as below:

OS: defined as the time from date of randomization to date of death due to any cause. Participants alive at the time of analysis were censored on the last date the participant was known to be alive.

Time to symptomatic progression: defined as time from date of randomization to the date of any of the following (whichever occurred first):
The use of external beam radiation for skeletal or pelvic symptoms. Note: Only radiation planned prior to randomization was not considered as symptomatic progression.
The need for tumor-related orthopedic surgical intervention.
Other cancer-related procedures (eg, nephrostomy insertion, bladder catheter insertion, or surgery for tumor symptoms)
Cancer-related morbid events (ie, fracture [symptomatic and/or pathologic], cord compression, urinary obstructive events)
Initiation of a new systemic anti-cancer therapy because of cancer symptoms.

Time to subsequent therapy: defined as the time from date of randomization to the date of initiation subsequent therapy for prostate cancer.

### Other Endpoints

Other endpoints included the following:
Patient-reported outcomes, including TTPP as defined as the time from the date of randomization to the date of deterioration in worst pain intensity (BPI question #3) observed at 2 consecutive evaluations ≥3 weeks apart.
PFS2—Time from date of randomization to date of first occurrence of disease progression (radiographic, clinical, or PSA progression) on first subsequent therapy for prostate cancer or death, whichever occured first
Objective response was defined as achieving a partial response or complete response according to modified RECIST 1.1.
Time to PSA progression was defined as the time from the date of randomization to the date of PSA progression based on PCWG3 criteria.
Objective response, rPFS, and PSA response across individual and subgroups of deleterious germline or somatic HRR gene alterations.

### Example 2—Primary Analysis (CCO: 7 Jan. 2025): Final Analysis for Primary Endpoint: Investigator-Assessed Radiographic Progression-Free Survival (rPFS). Interim Analysis 1 (IA1) for Secondary Endpoints: Time to Symptomatic Progression (TSP) and Overall Survival (OS)

Population—Gene subgroups: Primary and secondary end points were formally evaluated in HRR target subgroups: BRCA alterations (BRCA1, BRCA2), HRR Effectors (BRCA1, BRCA2, BRIP1, PALB2, RAD51B, RAD54L), and in All HRR subjects (BRCA1, BRCA2, BRIP1, CDK12, CHEK2, FANCA, PALB2, RAD51B, RAD54L).

Primary analysis set: The Full Analysis Set (FAS), including all participants classified according to their assigned treatment arm, was used to conduct efficacy, demographic, baseline disease characteristics, and disposition. The Safety Analysis Set (SAF), including all participants who received at least 1 dose of study medication, was used to conduct safety and exposure analyses.

Primary efficacy endpoint: The primary efficacy endpoint was investigator-assessed radiographic progression-free survival (rPFS), defined as time from randomization to radiographic progression (soft tissue progression by Response Evaluation Criteria In Solid Tumors v1.1 and/or bone progression defined by Prostate Cancer Working Group 3) or death, whichever came first.

Secondary efficacy endpoints: Secondary endpoints are overall survival (OS), time to symptomatic progression (TSP), and time to subsequent therapy (TST) for prostate cancer.

The primary analysis: The primary analysis includes the single formal analysis for rPFS (i.e. final analysis of the primary efficacy endpoint) and the first interim analysis of the secondary endpoints.

Interim analysis for secondary endpoints: Two interim analyses (IA1, IA2) and a final analysis were planned for OS. Formal analyses for TSP were to be performed at IA1 and IA2 only. IA1 coincided with the timing of the primary efficacy endpoint analysis.

Analysis—Testing Strategy: The SAP provided details on the graphical testing framework with group sequential design that was used to strictly control the familywise type 1 error rate at 2-sided alpha at 0.05 level. The level of significance to be used for each endpoint, based on the null hypotheses which were rejected and the proportion of events observed, was provided in each corresponding result section.

### Primary and Secondary Objective(s)

The primary objective is to determine if niraparib and AAP compared with AAP in participants with HRR gene-mutated mCSPC provides superior efficacy in improving rPFS. The secondary objectives are to assess the clinical benefit (OS, TSP, TST) of niraparib and AAP compared with AAP in participants with HRR gene-mutated mCSPC and to characterize its safety profile.

### Topline Results Summary

A statistically significant and clinically meaningful benefit in rPFS for the combination of niraparib and abiraterone acetate in mHSPC was demonstrated in the BRCA, HRR Effectors, and All HRR populations, supported by statistically significant benefit in TSP, and a trend in OS across all subgroups. The safety profile was consistent with prior experience from the MAGNITUDE study and the known safety profile of the single agents.

### Demographics:

This double-blind, placebo-controlled, multicenter study randomized 696 subjects overall from December 2020 to July 2023, 348 into each treatment arm. At the time of Clinical Cut-Off (CCO, 7 Jan. 2025) 69% of subjects were still on study, and 49% were continuing study treatment (54% in the nira+AAP arm and 44% in the placebo+AAP arm).

Demographics and baseline characteristics in the All HRR population were generally balanced between arms. The BRCA and HRR Effectors subgroups were mostly similar to the All HRR population and generally balanced between arms. The median age was 68 years (range: 40 to 88) in the nira+AAP arm and 67 years (range: 40 to 92) in the placebo+AAP arm. Most subjects were white (72.3%), had high volume disease (77.6%) and no prior docetaxel (84.2%).

### Efficacy:

Following the hierarchy of testing in the graphical testing framework, efficacy results for the primary and key secondary endpoints were presented to match the hypothesis testing order (first in the BRCA subgroup, HRR Effectors subgroup, and then All HRR population) followed by other secondary endpoints.

With an overall 30.8 months median follow-up in All HRR, the data for OS were not fully mature at IA1.

Efficacy data in the BRCA subgroup, the HRR Effectors subgroup and the All HRR population, are summarized in Table 4 and detailed in sections below (primary efficacy endpoint and secondary efficacy endpoints).

TABLE 4
Summary of Primary and Secondary Efficacy Endpoints

| | | | | | |
|---|---|---|---|---|---|
| | | | | Stratified | Prespecified |
| | Placebo + | Nira + | | 2-sided | Significance |
| | AAP | AAP | | Log-rank | Boundary |
| | Median | Median | Hazard ratio\hat{ } | Test | per Graph. |
| Analysis set: FAS | (in months) | (in months) | (95% CI) | p-value | Approach |

Radiographic Progression-free Survival (Final Analysis)

| | | | | | |
|---|---|---|---|---|---|
| BRCA | 26.0 | NR | 0.52 (0.37, 0.72) | <0.0001* | 0.05 |
| HRR Effectors | 27.6 | NR | 0.57 (0.42, 0.77) | 0.0003* | 0.025 |
| All HRR | 29.5 | NR | 0.63 (0.49, 0.80) | 0.0001* | 0.02475 |

Time to Symptomatic Progression (Interim Analysis 1)

| | | | | | |
|---|---|---|---|---|---|
| BRCA | NR | NR | 0.44 (0.29, 0.68) | 0.0001* | 0.03426 |
| HRR Effectors | NR | NR | 0.49 (0.33, 0.74) | 0.0004* | 0.02977 |
| All HRR | NR | NR | 0.50 (0.36, 0.69) | <0.0001* | 0.03207 |

Overall Survival (Interim Analysis 1)

| | | | | | |
|---|---|---|---|---|---|
| BRCA | NR | NR | 0.75 (0.51, 1.11) | 0.1454 | 0.007047 |
| HRR Effectors | NR | NR | 0.81 (0.57, 1.16) | 0.2485 | 0.000034 |
| All HRR | NR | NR | 0.79 (0.59, 1.04) | 0.0951 | 0.000043 |

Time to Subsequent Therapy

| | | | | | |
|---|---|---|---|---|---|
| BRCA | 30.0 | NV | 0.47 (0.33, 0.66) | <0.0001 | nominal |
| HRR Effectors | 33.6 | NV | 0.50 (0.36, 0.69) | <0.0001 | nominal |
| All HRR | NR | NV | 0.54 (0.41, 0.70) | <0.0001 | nominal |

NR = Not Reached;
NV = Not valid - KM plot artefact, a subject in the 3 subjects with longest follow-up is an event;
\hat{ }Stratified Cox regression model;
*Statistically significant result

In the BRCA subgroup (387 subjects), treatment with nira+AAP resulted in statistically significant and clinically meaningful improvement in the primary endpoint of investigator-assessed rPFS compared to the placebo+AAP arm (median 26 months vs not reached, HR=0.52; 95% CI: 0.37,0.72; p-value<0.0001) and in TSP (HR=0.44; 95% CI: 0.29,0.68; p-value=0.0001). The benefit in rPFS was supported by a strong trend for OS improvement (HR-0.75; 95% CI: 0.51,1.11, p-value=0.1454) and clinically meaningful improvement in TST (HR=0.47; 95% CI: 0.33,0.66; nominal p-value=<0.0001). The 2-sided log-rank test stratified by the volume of disease was used for those analyses.

In HRR Effectors subgroup (456 subjects), treatment with nira+AAP resulted in statistically significant and clinically meaningful improvement of investigator-assessed rPFS compared to those in the placebo+AAP arm (median 27.6 months vs not reached, HR=0.57; 95% CI: 0.42,0.77; p=value=0.0003) and in TSP (HR=0.50; 95% CI: 0.33,0.74; p-value=0.0004). The benefit in rPFS was supported by a trend for OS improvement (HR-0.81; 95% CI: 0.57,1.16; p-value=0.2485) and clinically meaningful improvement in TST (HR=0.50; 95% CI: 0.36,0.69; nominal p-value=<0.0001). The 2-sided log-rank test stratified by the volume of disease was used for those analyses.

In All HRR population (696 subjects), treatment with nira+AAP resulted in statistically significant and clinically meaningful improvement of investigator-assessed rPFS compared to those in the placebo+AAP arm (median 29.5 months vs not reached, HR=0.63; 95% CI: 0.49,0.80; p=value=0.0001) and in TSP (HR-0.50; 95% CI: 0.36,0.69; p-value<0.0001). The benefit in rPFS was supported by a trend for OS improvement (HR=0.79; 95% CI: 0.59,1.04; p-value=0.0951) and clinically meaningful improvement in TST (HR=0.54; 95% CI: 0.41,0.70; nominal p-value=<0.0001). The 2-sided log-rank test stratified by the volume of disease and gene status at study entry (BRCA2 versus all other pathogenic alterations) was used for those analyses.

Although the study was not powered to test for treatment difference in the Non-BRCA subgroup (309 subjects), treatment with nira+AAP resulted in clinically meaningful improvement in the investigator-assessed rPFS compared to those in the placebo+AAP arm (HR=0.81; 95% CI: 0.56,1.18; nominal p=0.2749). In addition, treatment with nira+AAP resulted in clinically meaningful improvements in TSP (HR=0.59; 95% CI: 0.36,0.98; nominal p-value=0.0385). The benefit in rPFS was supported by a trend for OS improvement (HR=0.83; 95% CI: 0.55,1.26; nominal p=0.3799) and a clinically meaningful improvement in TST (HR=0.68; 95% CI: 0.45,1.04; nominal p=0.0767). The 2-sided log-rank test stratified by the volume of disease was used for those analyses.

### Safety:

Overall, the safety profile of nira+AAP in the mCSPC setting was consistent with the known safety profile of the nira+AAP combination in mCRPC based on the MAGNITUDE study. The most common treatment-emergent adverse events (PT, more than 20%) were anemia (51.6%), hypertension (43.9%), constipation (35.2%), nausea (30.8%), fatigue (26.2%), hypokalemia (25.9%), neutropenia (21.9%), and arthralgia (21.0%). The regimen of nira+AAP was tolerable despite the high baseline disease burden in an elderly population, of whom 77.3% had high volume disease and where 24.1% were over 75 years of age. AEs were generally manageable with dose modifications and supportive care. The majority of subjects were able to continue treatment until disease progression with only 11.0% discontinuing treatment due to an AE.

### Conclusion:

AMPLITUDE is a multicenter, international, double-bind, randomized trial with an active standard of care comparator arm having enrolled a large sample of subjects with HRR, including BRCA alterations, with mCSPC.

The primary efficacy analysis of AMPLITUDE demonstrated the benefit of nira+AAP in patients with mCSPC and HRR alterations, particularly in the BRCA subpopulation. This was evidenced by statistically significant and clinically meaningful improvements in rPFS and TSP with a trend for improvement in OS at this early stage across the tested populations. In particular for the BRCA population, a reduction in risk of ~50% was observed in rPFS and TSP and an unequivocal trend in OS.

This was the first phase 3 study in the mCSPC population with HRR alterations to demonstrate efficacy with combination of PARPi with an androgen receptor pathway inhibitor. This also highlighted the importance of a priori testing for HRR gene alterations in patients with mCSPC to identify those who will derive optimal benefit from the combination of nira+AAP.

The safety profile in the mCSPC population was consistent with the known profile of the nira+AAP combination and AEs were manageable with dose modifications and supportive care when applicable.

Overall, the primary analysis of the AMPLITUDE data strongly supported the benefit of nira+AAP for the treatment of patients with mCSPC with HRR alterations, and in particular BRCA alterations, representing a new standard of care in these populations.

### Primary Endpoint Analysis

### Radiographic Progression-free Survival in BRCA

The primary efficacy endpoint was investigator-assessed rPFS defined as time from randomization to radiographic progression (soft tissue progression by RECIST v1.1 and/or bone progression defined by Prostate Cancer Working Group 3) or death, whichever came first.

This primary analysis included the final analysis for rPFS. Following the hierarchy of testing, the type I error for the rPFS analysis was 0.05 in the BRCA subgroup, 0.025 in HRR Effectors subgroup, and 0.02475 in All HRR population. The efficacy results for the primary endpoint were presented in this order: BRCA subgroup (Hypothesis 1), HRR Effectors subgroup (Hypothesis 2), All HRR population (Hypothesis 3).

The 2-sided log-rank test stratified by the volume of disease was used for the rPFS analysis in the BRCA and HRR Effectors subgroups, and by the volume of disease and gene status at study entry (BRCA2 versus all other pathogenic alterations) in All HRR population.

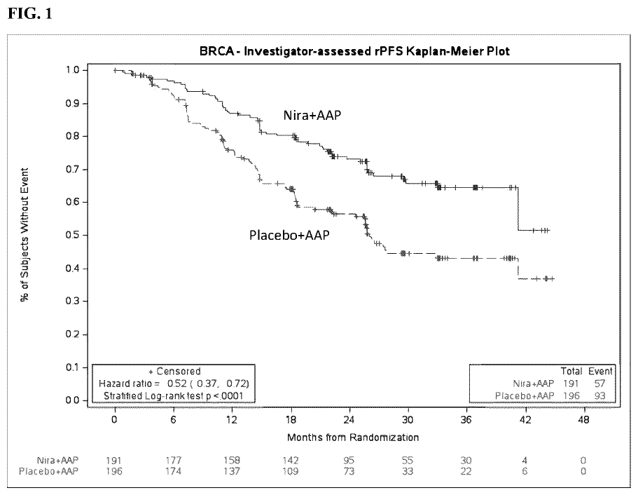

There was a statistically significant and clinically meaningful benefit in rPFS for subjects with BRCA gene alterations in the nira+AAP arm (median not reached) compared to those in the placebo+AAP arm (median: 26 months), with a 48% reduction in the risk of radiographic progression or death with nira+AAP treatment as shown on FIG. 1 (stratified 2-sided log-rank p-value of <0.0001, which is below the prespecified significance boundary of 0.05).

### Radiographic Progression-Free Survival in HRR Effectors

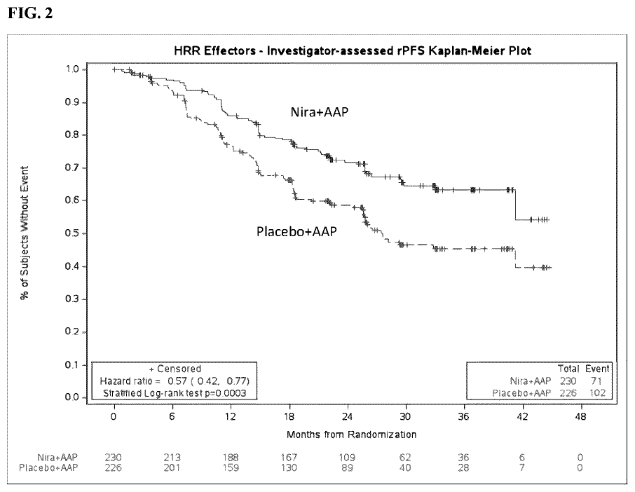

There was a statistically significant and clinically meaningful benefit in rPFS for subjects with HRR Effectors gene alterations in the nira+AAP arm (median not reached) compared to those in the placebo+AAP arm (median: 27.6 months), with a 43% reduction in the risk of radiographic progression or death with nira+AAP treatment as shown on the FIG. 2 (stratified 2-sided log-rank p-value of 0.0003, which is below the prespecified significance boundary of 0.025).

### Radiographic Progression-Free Survival in all HRR

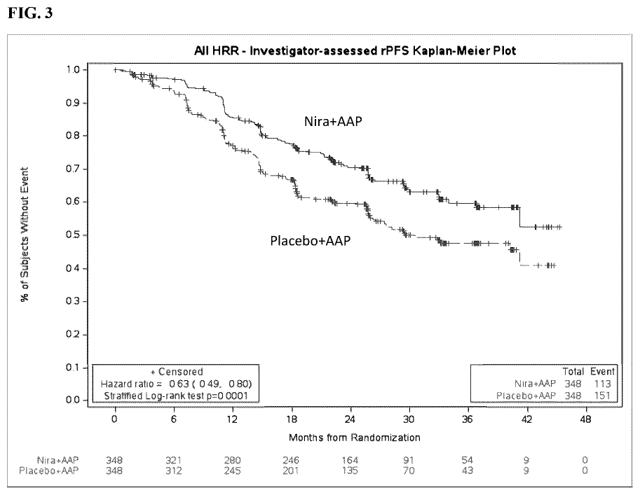

There was a statistically significant and clinically meaningful benefit in rPFS in the All HRR population in the nira+AAP arm (median not reached) compared to those in the placebo+AAP arm (median: 29.5 months), with a 37% reduction in the risk of radiographic progression or death with nira+AAP treatment as shown on the FIG. 3 (stratified 2-sided log-rank p-value of 0.0001, which is below the prespecified significance boundary of 0.02475).

### Secondary Endpoint(s) Analysis

The secondary efficacy endpoints were overall survival (OS), time to symptomatic progression (TSP), and time to subsequent therapy (TST) for prostate cancer.

This primary analysis included Interim Analysis 1 (IA1) of OS and TSP. Following the hierarchy of testing in the graphical approach with a group sequential design, the type I error for the TSP and OS analysis were dependent on the testing order, the results at prior tests and the proportion of events observed. The level of significance to be used for each endpoint was provided in each corresponding result section. The efficacy results for TSP were presented first and in the following order: BRCA subgroup, HRR Effectors subgroup, and All HRR population; then similarly for OS in the BRCA subgroup, HRR Effectors subgroup, and All HRR population.

With an overall 30.8 months median follow-up in All HRR, the data for TSP and OS were not fully mature at IA1.

The 2-sided log-rank test stratified by the volume of disease was used for the OS, TSP and TST analyses in the BRCA and HRR Effectors subgroups, and by the volume of disease and gene status at study entry (BRCA2 versus all other pathogenic alterations) in the OS, TSP and TST analyses in the All HRR population.

Time to symptomatic progression was defined as time from the date of randomization to the date of any of the following (whichever occured first):
The use of external beam radiation therapy for skeletal or pelvic symptoms.
The need for tumor-related orthopedic surgical intervention
Other cancer-related procedures (eg, nephrostomy insertion, bladder catheter insertion, external beam radiation therapy, or surgery for tumor symptoms)
Cancer-related morbid events (ie, fracture [symptomatic and/or pathologic], cord compression, urinary obstructive events)
Initiation of a new systemic anti-cancer therapy because of cancer symptoms.

### Time to Symptomatic Progression in BRCA

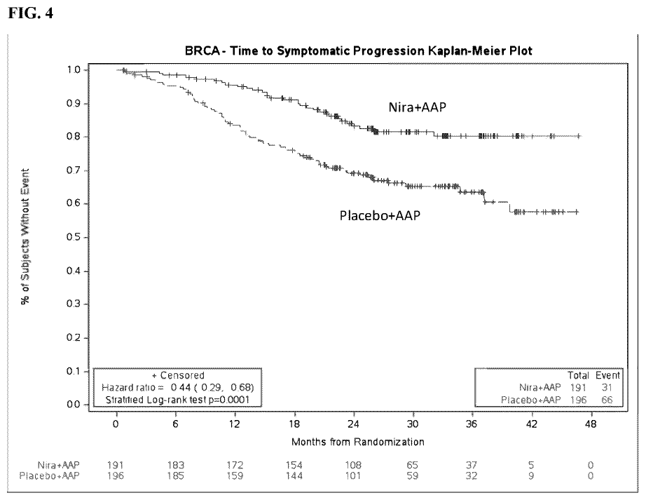

A statistically significant and clinically meaningful prolongation in TSP was observed in the nira+AAP arm (median not reached) compared to the placebo+AAP arm (median not reached), with a 56% reduction in the risk of symptomatic progression with nira+AAP treatment (FIG. 4).

### Time to Symptomatic Progression in HRR Effectors

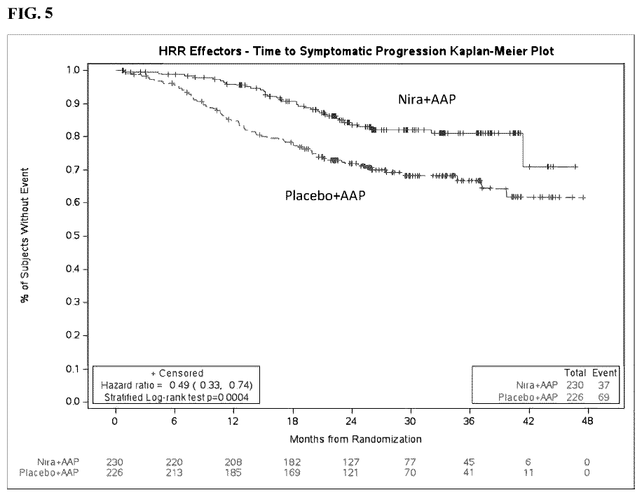

A statistically significant and clinically meaningful prolongation in TSP was observed in the nira+AAP arm (median not reached) compared to the placebo+AAP arm (median not reached), with a 51% reduction in the risk of symptomatic progression with nira+AAP treatment (FIG. 5).

### Time to Symptomatic Progression in All HRR

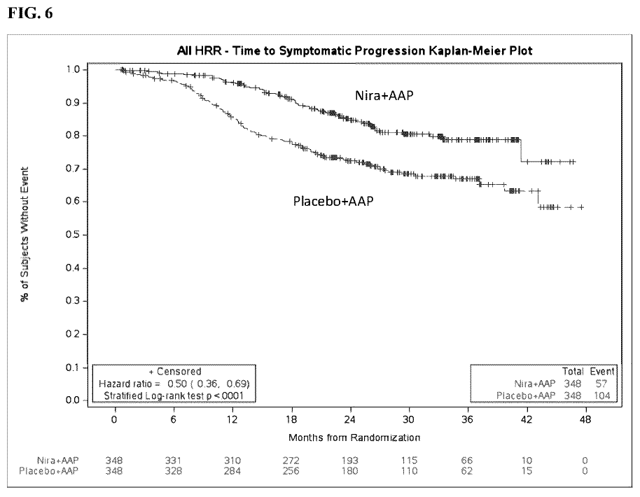

A statistically significant and clinically meaningful prolongation in TSP was observed in the nira+AAP arm (median not reached) compared to the placebo+AAP arm (median not reached), with a 50% reduction in the risk of symptomatic progression with nira+AAP treatment (FIG. 6).

### Overall Survival in BRCA

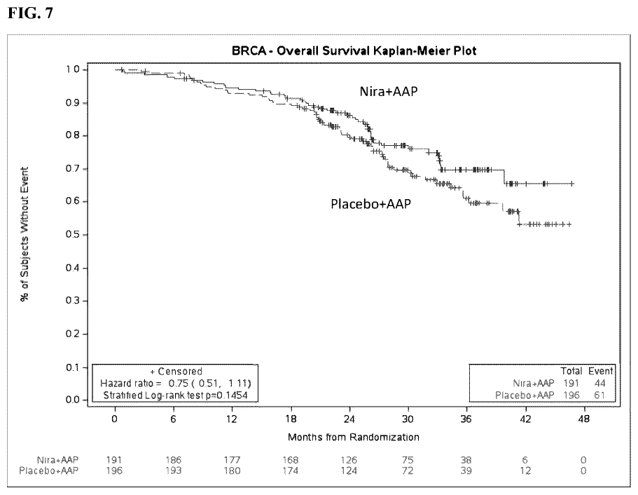

Median OS was not reached in either arm. The difference between arms showed a strong trend of improvement in the nira+AAP arm compared to the placebo+AAP arm with a 25% reduction in the risk of death with nira+AAP treatment (FIG. 7) which did not meet statistical significance at IA1 with a p-value of 0.145 above the prespecified significance boundary of 0.007047 for IA1.

### Overall Survival in HRR Effectors

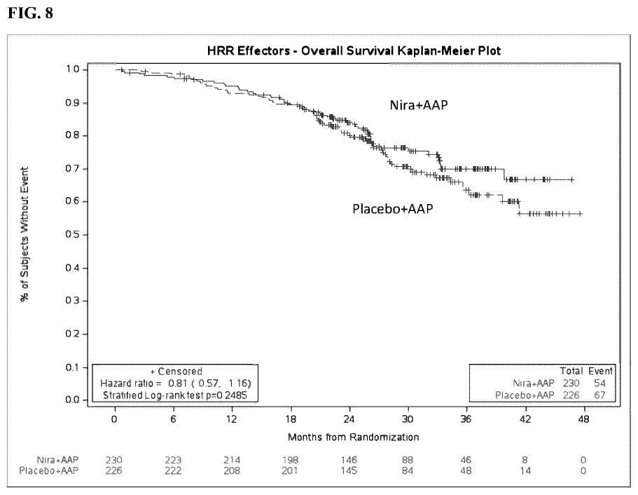

Median OS was not reached in either arm. The difference between arms showed a trend of improvement in the nira+AAP arm compared to the placebo+AAP arm with a 19% reduction in the risk of death with nira+AAP treatment (FIG. 8) which did not meet statistical significance at IA1 with a p-value of 0.2485 above the prespecified significance boundary of 0.000034 for IA1.

### Overall Survival in all HRR Population

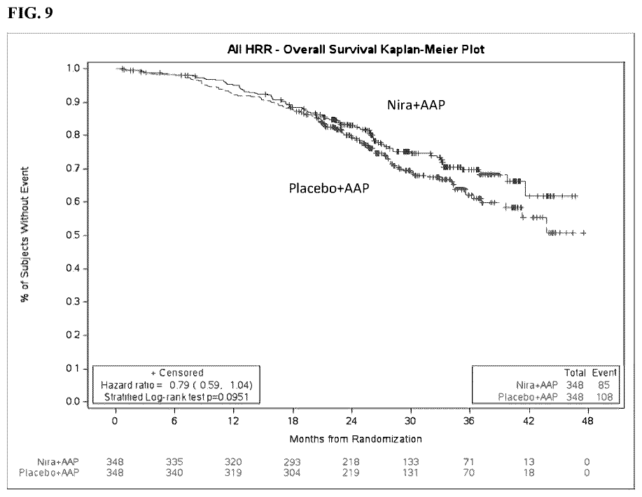

Median OS was not reached in either arm. The difference between arms showed a trend of improvement in the nira+AAP arm compared to the placebo+AAP arm with a 21% reduction in the risk of death with nira+AAP treatment (FIG. 9) which did not meet statistical significance at IA1 with a p-value of 0.0951 above the prespecified significance boundary of 0.000043 for IA1.

### Example 3—Interim Analysis 2 (IA2) (CCO: 3 Oct. 2025) Results

Primary efficacy endpoint: The primary efficacy endpoint was investigator-assessed radiographic progression-free survival (rPFS), defined as time from randomization to radiographic progression (soft tissue progression by Response Evaluation Criteria In Solid Tumors v1.1 and/or bone progression defined by Prostate Cancer Working Group 3) or death, whichever came first.

The primary analysis and interim analyses for secondary endpoints: The primary analysis (PA-IA1, with a CCO date of 7 Jan. 2025) included the single formal analysis for rPFS (i.e. final analysis of the primary efficacy endpoint) and the first interim analysis (IA1) for secondary endpoints. Two interim analyses (IA1, IA2) and a final analysis were planned for OS. Formal analyses for TSP were planned at IA1 and IA2 only. IA1 coincided with the timing of the primary efficacy endpoint analysis. A statistically significant and clinically meaningful benefit in rPFS and TSP for the combination of niraparib and abiraterone acetate in mHSPC was demonstrated in all study populations: the BRCA and the HRR Effectors subgroups, and the All HRR population at that first analysis.

Analysis—Testing Strategy: The SAP provided details on the graphical testing framework with group sequential design that was used to strictly control the familywise type 1 error rate at 2-sided alpha at 0.05 level. The level of significance to be used for each endpoint, based on the null hypotheses which were rejected and the proportion of events observed, was provided in each corresponding result section.

### Primary and Secondary Objective(s)

The primary objective, to determine if niraparib and AAP compared with AAP in participants with HRR gene-mutated mHSPC provided superior efficacy in improving rPFS, was met at the primary analysis.

The secondary objective, to assess the clinical benefit (TSP, TST) of niraparib and AAP compared with AAP in participants with HRR gene-mutated mHSPC and to characterize its safety profile, was also met at the time of the first interim analysis (primary analysis for rPFS).

### Topline Results Summary

At the time of the primary analysis (PA-IA1), a statistically significant and clinically meaningful benefit in rPFS and TSP for the combination of niraparib and abiraterone acetate in mHSPC was demonstrated in all study populations: the BRCA subpopulation, the HRR Effectors subpopulation, and the All HRR population. Therefore, this second planned interim analysis consisted of an update of the primary (rPFS) and other secondary endpoints.

### Demographics:

A total of 696 participants were randomized from December 2020 to July 2023, 348 into each treatment arm. BRCA gene alteration was observed in 387 participants. At the time of CCO for IA2 (3 Oct. 2025) 41.6% of participants remained on study treatment (47.0% in the Nira+AAP arm and 36.2% in the PBO+AAP arm) and 58.9% of participants were still on study for follow-up.

Demographics and baseline characteristics in the All HRR population remain unchanged from IA1. They were generally balanced between arms. The BRCA and HRR Effectors subgroups were mostly similar to the All HRR population and generally balanced between arms. The median age was 68 years (range: 40 to 88) in the Nira+AAP arm and 67 years (range: 40 to 92) in the PBO+AAP arm. Most participants were white (72.3%), had high volume disease (77.6%) and had no prior docetaxel (84.2%).

### Efficacy:

Following the hierarchy of testing in the graphical testing framework, OS results were presented to match the hypothesis testing order, first in the BRCA subgroup, then in the HRR Effectors subgroup, and finally in the All HRR population. Tests were performed according to the prespecified significance boundary as defined per the group sequential design within the graphical approach.

With an overall 40.5 months median follow-up in the All HRR population, 67.1% (261/389) of the total number of events anticipated at final OS analyses were observed at IA2, that is 68 OS events more than at IA1.

With statistically significant results already achieved overall in the All HRR population at IA1, an update of the efficacy results for rPFS and TSP, as well as TST was provided for assessing the consistency of the results with further follow-up. In particular, the statistically significant rPFS and TSP results in the BRCA subgroup, the HRR Effectors subgroup, and the All HRR population, were consistent with IA1 results.

TABLE 5
Summary of Primary and Secondary
Efficacy Endpoints at IA1 and IA2

| | | | | |
|---|---|---|---|---|
| | Placebo + | Nira + | | Stratified |
| | AAP | AAP | | 2-sided |
| | Median | Median | Hazard | Log-rank |
| | (in | (in | ratio\hat{ } | Test |
| Analysis set: FAS | months) | months) | (95% CI) | p-value |

Radiographic Progression-free Survival
(Updated analysis at time of Interim Analysis 2)

| | | | | |
|---|---|---|---|---|
| BRCA | 25.8 | NR | 0.49 (0.36, 0.67) | <0.0001^{#} |
| HRR Effectors | 27.6 | NR | 0.58 (0.44, 0.76) | <0.001^{#} |
| All HRR | 29.6 | 48.8 | 0.62 (0.49, 0.77) | <0.0001^{#} |
| Non-BRCA | 40.2 | 44.2 | 0.81 (0.58, 1.14) | 0.23# |

(Primary Analysis)

| | | | | |
|---|---|---|---|---|
| BRCA | 26.0 | NR | 0.52 (0.37, 0.72) | <0.0001* |
| HRR Effectors | 27.6 | NR | 0.57 (0.42, 0.77) | 0.0003* |
| All HRR | 29.5 | NR | 0.63 (0.49, 0.80) | 0.0001* |
| Non-BRCA | 40.2 | NR | 0.81 (0.56, 1.18) | 0.27^{#} |

Time to Symptomatic Progression
(Updated Analysis at time of Interim Analysis 2)

| | | | | |
|---|---|---|---|---|
| BRCA | NR | NR | 0.46 (0.31, 0.69) | 0.0001^{#} |
| HRR Effectors | NR | NR | 0.52 (0.35, 0.75) | 0.0004^{#} |
| All HRR | NR | NR | 0.51 (0.38, 0.69) | <0.0001^{#} |
| Non-BRCA | NR | NR | 0.58 (0.37, 0.93) | 0.022^{#} |

(Interim Analysis 1)

| | | | | |
|---|---|---|---|---|
| BRCA | NR | NR | 0.44 (0.29, 0.68) | 0.0001* |
| HRR Effectors | NR | NR | 0.49 (0.33, 0.74) | 0.0004* |
| All HRR | NR | NR | 0.50 (0.36, 0.69) | <0.0001* |
| Non-BRCA | NR | NR | 0.59 (0.36, 0.98) | 0.039^{#} |

Time to Subsequent Therapy
(Updated analysis at time of Interim Analysis 2)

| | | | | |
|---|---|---|---|---|
| BRCA | 28.6 | NR | 0.41 (0.30, 0.57) | <0.0001^{#} |
| HRR Effectors | 33.6 | NR | 0.47 (0.35, 0.64) | <0.0001^{#} |
| All HRR | 36.1 | NR | 0.52 (0.40, 0.66) | <0.0001* |
| Non-BRCA | NR | NR | 0.73 (0.50, 1.08) | 0.12^{#} |

(Analysis at time of Interim Analysis 1)

| | | | | |
|---|---|---|---|---|
| BRCA | 30.0 | NV | 0.47 (0.33, 0.66) | <0.0001^{#} |
| HRR Effectors | 33.6 | NV | 0.50 (0.36, 0.69) | <0.0001^{#} |
| All HRR | NR | NV | 0.54 (0.41, 0.70) | <0.0001^{#} |
| Non-BRCA | NR | NR | 0.68 (0.45, 1.04) | 0.38^{#} |

NR = Not Reached
NV = Not valid - KM plot artifact: a participant of the last 5 participant at risk was an event
\hat{ }Stratified Cox regression model
*Formal statistically significant result (ie. included in the overall 0.05 alpha)
^{#}Nominal p-value

In the BRCA subgroup (n=387), at IA2 a trend for OS improvement (HR=0.80, 95% CI: 0.58,1.11; p-value=0.18) with Nira+AAP compared to the PBO+AAP was observed, consistent with the results reported for IA1. Statistically significant improvement in rPFS was demonstrated at PA-IA1. The updated analysis at IA2 further corroborated the conclusion drawn at IA1, that treatment with Nira+AAP results in clinically meaningful improvement of investigator-assessed rPFS compared to the PBO+AAP arm (median not reached vs 25.8 months, HR=0.49, 95% CI: 0.36,0.67; nominal p-value<0.0001), as well as in TSP (HR=0.46, 95% CI: 0.31,0.69; nominal p-value=0.0001) and in TST (HR=0.41, 95% CI: 0.30,0.57; nominal p-value<0.0001). The 2-sided log-rank test stratified by the volume of disease was used for those analyses.

In the HRR Effectors subgroup (n=456), at IA2 the trend for OS improvement continues to be observed (HR=0.87, 95% CI: 0.64,1.17; p-value=0.35) with Nira+AAP compared to the PBO+AAP, consistent with the results reported for IA1. Benefit in rPFS was demonstrated at IA1. The updated IA2 analysis corroborated the clinically meaningful improvement of investigator-assessed rPFS with Nira+AAP compared to PBO+AAP (median not reached vs 27.6 months, HR=0.58, 95% CI: 0.44,0.76; nominal p-value=<0.0001) as well as the benefit observed in TSP (HR=0.52, 95% CI: 0.35,0.75; nominal p-value=0.0004) and TST (HR=0.47, 95% CI: 0.35,0.64; nominal p-value<0.0001). The 2-sided log-rank test stratified by the volume of disease was used for those analyses.

In the All HRR population (n=696), at IA2 a trend for OS improvement (HR=0.82, 95% CI: 0.65,1.05; p-value=0.12) with Nira+AAP compared to the PBO+AAP was observed, consistent with the results reported for IA1. The benefit in rPFS was demonstrated at IA1 and was observed in the updated IA2 analysis, resulting in an over 19-month improvement in the median (median of 48.8 months in Nira+AAP vs 29.6 months in PBO+AAP, HR-0.62, 95% CI: 0.49,0.77 nominal p-value<0.001). Benefit was also observed in TSP (HR=0.51, 95% CI: 0.38,0.69; nominal p-value<0.0001) and in TST (HR=0.52, 95% CI: 0.40,0.66; nominal p-value<0.0001). The 2-sided log-rank test stratified by the volume of disease and gene status at study entry (BRCA2 versus all other pathogenic alterations) was used for those analyses.

Although the study was not powered to test for treatment difference in the Non-BRCA subgroup (n=309), at IA2, treatment with Nira+AAP resulted in clinically meaningful improvement in OS compared to PBO+AAP (HR=0.85, 95% CI: 0.59,1.22; nominal p-value=0.38). In addition, treatment with Nira+AAP resulted in clinically meaningful improvement in investigator-assessed rPFS compared to PBO+AAP (HR=0.81, 95% CI: 0.58,1.14; nominal p-value=0.23), which was also observed in a sensitivity analysis of rPFS not censored for subsequent therapy (HR-0.73, 95% CI: 0.53,1.01; nominal p-value=0.056). Clinically meaningful improvement was also demonstrated in TSP (HR=0.58, 95% CI: 0.37,0.93; nominal p-value=0.022) and in TST (HR=0.73, 95% CI: 0.50,1.08; nominal p-value=0.12). The 2-sided log-rank test stratified by the volume of disease was used for those analyses.

### Conclusion

The primary efficacy analysis of AMPLITUDE demonstrated the benefit of Nira+AAP in participants with mHSPC and HRR alterations, particularly in the BRCA subpopulation. This was based on statistically significant and clinically meaningful improvements in rPFS and TSP across all tested subpopulations. The trend for improvement in OS, already apparent at IA1, was observed at this second interim analysis. A clinically meaningful benefit in OS (HR=0.82; p-value=0.12) for the combination of niraparib and abiraterone acetate in mHSPC was observed in the All HRR population, with a similar hazard ratio observed in the BRCA subgroup (HR=0.80; p-value=0.18). While the trial was not powered to test efficacy in individual genes or the Non-BRCA subgroup, the observed hazard ratios support the potential benefit of nira+AAP in patients harboring Non-BRCA gene alterations. In particular, the OS hazard ratio (HR=0.85; nominal p-value=0.38) was similar to that of the All HRR population.

The safety profile was consistent with the known profile of the Nira+AAP combination and AEs were managed with dose modifications and supportive care when applicable. About half of the participants continue study treatment.

This is the first phase 3 study in the mHSPC population with HRR alterations to demonstrate clear evidence of clinical benefit with the combination of a PARPi with an androgen receptor pathway inhibitor.

The second interim analysis of the AMPLITUDE data further supports a favorable benefit-risk profile of Nira+AAP for the treatment of patients with mHSPC and HRR alterations. The observed effects were most pronounced in the BRCA subgroup, and in particular BRCA2, and address the unmet medical need, providing a new standard of care in this population. Surprisingly, the non-BRCA subgroup also experienced better response with Nira+AAP, when compared to PBO+AAP, in terms of TSP. Also unexpectedly, the CHEK2 altered population with mCSPC also showed better response with Nira+AAP in terms of rPFS, when compared to PBO+AAP.

TABLE 6
Primary and Secondary Endpoint by Gene Alteration
Groupings - Stratified Analysis; Full Analysis Set

| | | rPFS PA | | rPFS IA2 | |
|---|---|---|---|---|---|
| | Treatment | N | | N | |
| Gene^{a} | group | (events) | HR(95% CI)^{b} | (events) | HR(95% CI)^{b} |
| BRCA2 | PBO + AAP | 163(83) | | 163(94) | |
| | Nira + AAP | 163(48) | 0.456 (0.319, 0.652) | 163(55) | 0.436 (0.312, 0.609) |
| | | TSP IA1 | | TSP IA2 | |
| | Treatment | N | | N | |
| Gene^{a} | group | (events) | HR(95% CI)^{b} | (events) | HR(95% CI)^{b} |
| Non- | PBO + AAP | 152(38) | | 152(44) | |
| BRCA | | | | | |
| | Nira + AAP | 157(26) | 0.593 (0.360, 0.978) | 157(30) | 0.584 (0.367, 0.930) |

Key: AAP = abiraterone acetate plus prednisone, PBO = placebo, PA = primary analysis, IA1 = interim analysis 1, IA2 = interim analysis 2.
Key: rPFS = radiographic progression-free survival, TSP = time to symptomatic progression, CI = confidence interval, HR = hazard ratio, BRCA2 = breast cancer gene 2.
^{a}BRCA2: subjects with single or co-occurring BRCA2; Non-BRCA HRR Effectors: subjects with single or co-occurring BRIP1, PALB2, RAD51B, RAD54L alterations excluding those with BRCA co-occurring.
^{b}Hazard ratio is from stratified proportional hazards model by stratification factors: volume of disease (high versus low) and only for All HRR, the gene status (BRCA2 versus all other pathogenic alterations) is also used. Non estimable HRs are due to few or no events.

For the mCSPC BRCA2 altered population, consistent with IA1 results, treatment with Nira+AAP resulted in clinically meaningful improvement of investigator-assessed rPFS compared to the PBO+AAP arm (median not reached vs 25.6 months, HR=0.436, 95% CI: 0.312,0.609;), in TSP (HR=0.428, 95% CI: 0.279,0.658) and in TST (median not reached vs 26.3 months, HR=0.351, 95% CI: 0.245,0.504) (Table 6 and 7). A trend for OS improvement (HR=0.712, 95% CI: 0.500,1.014) was also observed for patients receiving with Nira+AAP compared to the PBO+AAP cohort.

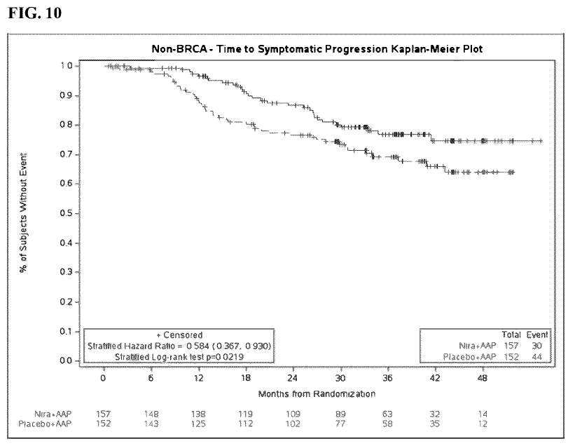

For the non-BRCA Subgroup, treatment with Nira+AAP resulted in improvement in TSP (FIG. 10; HR-0.584, 95% CI: 0.367,0.930; nominal p-value=0.0219; median not reached in both arms) with clear and sustained separation in the KM curves in this highly patient relevant endpoint.

TABLE 7
Radiographic Progression-free Survival by Investigator
Review Not Censored for Subsequent Therapy by Gene
Alteration Groupings - Stratified Analysis; Full Analysis Set
rPFS Not Censored for Subsequent Therapy

| | | N | Median | |
|---|---|---|---|---|
| Genea | Treatment group | (events) | (95% CI) | HR(95% CI)b |
| BRCA2 | PBO + AAP | 163(105) | 22.34 (17.35, 25.79) | |
| | Nira + AAP | 163(62) | NE (41.20, NE) | 0.451 (0.329, 0.619) |
| CHEK2 | PBO + AAP | 80(45) | 30.55 (18.33, 41.33) | |
| excluding: BRCA | | | | |
| | Nira + AAP | 76(30) | 47.87 (32.92, NE) | 0.550 (0.345, 0.877) |

aBRCA2: subjects with single or co-occurring BRCA2; CHEK2 excluding BRCA: includes subjects with single or co-occurring CHEK2 excluding co-occurring BRCA1 or BRCA2;

Because subsequent therapy does not censor the analysis, the “rPFS Not Censored for Subsequent Therapy” approach reflects the full duration until radiographic progression regardless of additional treatments, generally resulting in longer rPFS estimates. Table 7 provides a more “real-world” estimate of disease control, while Table 6 aligns with the protocol-defined primary endpoint.

### Example 4: Dec. 12, 2025—Letter from the Food and Drug Administration (FDA) Approving the Drug Product

### NDA 216793/S-001

### Supplement Approval

Janssen Biotech, Inc.
Attention: Elise D Mazzola, MPH
Director, Global Regulatory Affairs
920 U.S. Highway 202, P.O. Box 300
Raritan, NJ 08869

Dear Elise D Mazzola:

Please refer to your supplemental new drug application (sNDA) received Jun. 23, 2025, submitted under section 505(b) of the Federal Food, Drug, and Cosmetic Act (FDCA) for Akeega (niraparib/abiraterone acetate fixed-dose combination) 50/500 mg and 100/500 mg film-coated tablets.

This Prior Approval supplemental new drug application provides for Akeega (niraparib/abiraterone acetate fixed-dose combination) 50/500 mg and 100/500 mg film-coated tablets indicated with prednisone for adults with deleterious or suspected deleterious BRCA2-mutated metastatic castration-sensitive prostate cancer.

### Approval & Labeling

We have completed our review of this application. It is approved, effective on the date of this letter, for use as recommended in the enclosed agreed-upon labeling with minor editorial revisions reflected in the enclosed labeling.

### Content of Labeling

As soon as possible, but no later than 14 days from the date of this letter, submit the content of labeling [21 CFR 314.50 (0) in structured product labeling (SPL) format using the FDA automated drug registration and listing system (eLIST), as described at FDA.gov.¹ Content of labeling must be identical to the enclosed labeling (text for the Prescribing Information and Patient Package Insert), with the addition of any labeling changes in pending “Changes Being Effected” (CBE) supplements, as well as annual reportable changes not included in the enclosed labeling. ¹ http-www-fda.gov/ForIndustry/DataStandards/StructuredProductLabeling/default.htm

Information on submitting SPL files using eList may be found in the guidance for industry SPL Standard for Content of Labeling Technical Qs and As.²

The SPL will be accessible from publicly available labeling repositories.

Also within 14 days, amend all pending supplemental applications that include labeling changes for this NDA, including CBE supplements for which the FDA has not yet issued an action letter, with the content of labeling [21 CFR 314.50(I)(1)0A in Microsoft Word format, that includes the changes approved in this supplemental application, as well as annual reportable changes. To facilitate review of your submission(s), provide a highlighted or marked-up copy that shows all changes, as well as a clean Microsoft Word version. The marked-up copy should provide appropriate annotations, including supplement number(s) and annual report date(s).

### Required Pediatric Assessments

Under the Pediatric Research Equity Act (PREA) (21 U.S.C. 355c), all applications for new active ingredients (which includes new salts and new fixed combinations), new indications, new dosage forms, new dosing regimens, or new routes of administration are required to contain an assessment of the safety and effectiveness of the product for the claimed indication in pediatric patients unless this requirement is waived, deferred, or inapplicable.

We are waiving the pediatric study requirement for this application because necessary studies are impossible or highly impracticable.

### Postmarketing Commitments Subject to Reporting Requirements Under Section 506B

We remind you of your postmarketing commitments:
4943-1 Complete the trial AMPLITUDE (NCT04497844), which is an ongoing phase 3, randomized, double-blind, placebo-controlled clinical trial evaluating the efficacy and safety of niraparib in combination with abiraterone acetate in patients with homologous recombination repair gene-mutated metastatic castration-sensitive prostate cancer, to obtain the trial's final overall survival (OS) analysis. ² We update guidances periodically. For the most recent version of a guidance, check the FDA Guidance Documents Database http-www-fda.gov/RegulatoryInformation/Guidances/default.htm

The timetable you submitted on Nov. 19, 2025, states that you will conduct this study according to the following schedule:
Trial Completion: 12/2027
Final Report Submission: 06/2028
U.S. Food and Drug Administration
Silver Spring, MD 20993
www-fda.gov
Reference ID: 5710704
NDA 216793/S-001
Page 3
   4943-2 Conduct an analytical and clinical validation study using clinical trial data, adequate to support the availability of an in vitro diagnostic device using tissue samples that is essential to the safe and effective use of niraparib and abiraterone acetate (AKEEGA) for patients diagnosed with metastatic castration-sensitive prostate cancer (mCSPC), whose tumors harbor BRCA2 mutations.
The timetable you submitted on Nov. 19, 2025, states that you will conduct this study according to the following schedule:
   Final Report Submission: 03/2027
Submit clinical protocols to your IND 131190 for this product. Submit nonclinical and chemistry, manufacturing, and controls protocols and all postmarketing final reports to this NDA. In addition, under 21 CFR 314.81 (b)(2)(vii) and 314.81(b)(2)(viii) you should include a status summary of each commitment in your annual report to this NDA. The status summary should include expected summary completion and final report submission dates, any changes in plans since the last annual report, and, for clinical studies/trials, number of patients/subjects entered into each study/trial. All submissions, including supplements, relating to these postmarketing commitments should be prominently labeled “Postmarketing Commitment Protocol, “Postmarketing Commitment Final Report;” or “Postmarketing Commitment Correspondence.”

### Promotional Materials

You may request advisory comments on proposed introductory advertising and promotional labeling. For information about submitting promotional materials, see the final guidance for industry Providing Regulatory Submissions in Electronic and Non-Electronic Format-Promotional Labeling and Advertising Materials for Human Prescription Drugs.³

You must submit final promotional materials and Prescribing Information, accompanied by a Form FDA 2253, at the time of initial dissemination or publication [21 CFR 314.81(b)(3)(i)]. Form FDA 2253 is available at FDA.gov.⁴ Information and Instructions for completing the form can be found at FDA.gov.⁵

All promotional materials that include representations about your drug product must be promptly revised to be consistent with the labeling changes approved in this supplement, including any new safety-related information [21 CFR 314.70 (a) (4)]. The revisions in your promotional materials should include prominent disclosure of the important new safety-related information that appears in the revised labeling. Within 7 days of receipt of this letter, submit your statement of intent to comply with 21 CFR 314.70 (a) (4).

### Patent Listing Requirements

Pursuant to 21 CFR 314.53(d)(2) and 314.70(f), certain changes to an approved NDA submitted in a supplement require you to submit patent information for listing in the Orange Book upon approval of the supplement. You must submit the patent information required by 21 CFR 314.53(d)(2)(i)(A) through (C) and 314.53(d)(2)ii(A) and (C), as applicable, to the FDA on Form FDA 3542 within 30 days after the date of approval of the supplement for the patent information to be timely filed (see 21 CFR 314.53(c)(2)(ii)). You also must ensure that any changes to your approved NDA that require the submission of a request to remove patent information from the Orange Book are submitted to the FDA at the time of approval of the supplement pursuant to 21 CFR 314.53(d)(2)(ii)(B) and 314.53(f)(2)(iv). ³ For the most recent version of a guidance, check the FDA guidance web page at https-www-fdamov/media/128163/download.⁴ http-www-fda.gov/downloads/AboutFDA/ReportsManualsForms/Forms/UCM083570.pdf⁵ http-www-fda.gov/downloads/AboutFDA/ReportsManualsForms/Forms/UCM375154.pdf

### Reporting Requirements

We remind you that you must comply with reporting requirements for an approved NDA (21 CFR 314.80 and 314.81).

Your product is a Part 3 combination product (21 CFR 3.2(e)); therefore, you must also comply with postmarketing safety reporting requirements for an approved combination product (21 CFR 4, Subpart B). Additional information on combination product postmarketing safety reporting is available at FDA.gov.⁶

If you have any questions, contact Anna Lananh Nguyen, PharmD, Regulatory Project Manager, via email at Lananh Nguyen@fda.hhs.gov.

Sincerely,

{See appended electronic signature page}
Daniel Suzman, MD
Deputy Division Director
Division of Oncology 1
Office of Oncologic Diseases
Center for Drug Evaluation and Research

### Enclosure(s):

Content of Labeling
Prescribing Information
Patient Package Insert ⁶ https-www-fda.gov/combination-products/guidance-regulatory-information/postmarketing-safety-reporting-combination-products

### Example 5—Drug Product Label Revised by the Food and Drug Administration (FDA)

### Highlights of Prescribing Information

These highlights do not include all the information needed to use AKEEGA safely and effectively. See full prescribing information for AKEEGA.

AKEEGA® (niraparib and abiraterone acetate) tablets, for oral use Initial U.S. Approval: 2023

### Recent Major Changes

Indications and Usage (1) 12/2025
Dosage and Administration (2.1, 2.2) 12/2025
Warnings and Precautions (5.1, 5.2, 5.3, 5.4) 12/2025

### Indications and Usage

AKEEGA is a combination of niraparib, a poly (ADP-ribose) polymerase (PARP) inhibitor, and abiraterone acetate, a CYP17 inhibitor indicated with prednisone for the treatment of adult patients with:
deleterious or suspected deleterious BRCA2-mutated (BRCA2m) metastatic castration-sensitive prostate cancer (mCSPC).
deleterious or suspected deleterious BRCA-mutated (BRCAm) metastatic castration-resistant prostate cancer (mCRPC).
Select patients for therapy based on an FDA-approved test for AKEEGA. (1, 2.1)

### Dosage and Administration

BRCA2m mCSPC:
The recommended dosage of AKEEGA is 200 mg niraparib/1,000 mg abiraterone acetate orally once daily in combination with 5 mg prednisone daily until disease progression or unacceptable toxicity. (2.2)

BRCAm mCRPC:
The recommended dosage of AKEEGA is 200 mg niraparib/1,000 mg
abiraterone acetate orally once daily in combination with 10 mg prednisone daily until disease progression or unacceptable toxicity. (2.2)
Patients receiving AKEEGA should also receive a gonadotropin-releasing hormone (GnRH) analog concurrently or should have had bilateral orchiectomy. (2.2)
Take AKEEGA on an empty stomach at least one hour before or two hours after food. (2.2)
For adverse reactions, consider interruption of treatment, dose reduction, or dose discontinuation. (2.3)

### Dosage Forms and Strengths

Tablets:
50 mg niraparib/500 mg abiraterone acetate (3)
100 mg niraparib/500 mg abiraterone acetate (3)

### Contraindications

None. (4)

### Warnings and Precautions

Myelodysplastic Syndrome/Acute Myeloid Leukemia (MDS/AML): MDS/AML, including a case with fatal outcome, has been observed in patients treated with AKEEGA. Monitor patients for hematological toxicity and discontinue if MDS/AML is confirmed. (5.1)
Myelosuppression: Test complete blood counts weekly for the first month, every two weeks for the next two months, monthly for the remainder of the first year, then every other month, and as clinically indicated. (2.3, 5.2)
Hypokalemia, Fluid Retention, and Cardiovascular Adverse Reactions: Monitor patients for hypertension, hypokalemia, and fluid retention at least weekly for the first two months, then once a month. Closely monitor patients whose underlying medical conditions might be compromised by increases in blood pressure, hypokalemia, or fluid retention. Control hypertension and correct hypokalemia before and during treatment with AKEEGA. (5.3)
Hepatotoxicity: Can be severe and fatal. Monitor liver function and modify, interrupt, or discontinue treatment as recommended. (2.3, 5.4)
Adrenocortical insufficiency: Monitor for symptoms and signs of adrenocortical insufficiency. Increased dosage of corticosteroids may be indicated before, during and after stressful situations. (5.5)
Hypoglycemia: Severe hypoglycemia has been reported when abiraterone acetate, a component of AKEEGA, was administered to patients receiving medications containing thiazolidinediones (including pioglitazone) or repaglinide. Monitor blood glucose in patients with diabetes during and assess if antidiabetic agent dose modifications are required. (5.6)
Increased fractures and mortality in combination with radium Ra 223 dichloride: Use of AKEEGA plus prednisone in combination with radium Ra 223 dichloride is not recommended. (5.7)
Posterior Reversible Encephalopathy Syndrome (PRES): PRES has been observed in patients treated with niraparib, a component of AKEEGA. Discontinue AKEEGA if PRES is confirmed. (5.8)
Embryo-Fetal Toxicity: AKEEGA can cause fetal harm. Advise males with female partners of reproductive potential to use effective contraception. (5.9, 8.1, 8.3)

### Adverse Reactions

The most common adverse reactions (≥20%), including laboratory abnormalities, are decreased hemoglobin, decreased lymphocytes, musculoskeletal pain, fatigue, decreased platelets, increased alkaline phosphatase, constipation, hypertension, nausea, decreased neutrophils, increased creatinine, increased potassium, decreased potassium, increased AST, fluid retention/edema, increased bilirubin, respiratory tract infection and arrhythmia. (6)

To report SUSPECTED ADVERSE REACTIONS, contact Janssen Biotech, Inc. at 1-800-526-7736 (1-800-JANSSEN) or FDA at 1-800-FDA-1088 or www.fda.gov/medwatch.

### Drug Interactions

Strong CYP3A4 Inducers: Avoid coadministration. (7.1)
CYP2D6 Substrates: Avoid coadministration of AKEEGA with CYP2D6 substrates for which minimal changes in concentration may lead to serious toxicities. If alternative treatments cannot be used, consider a dose reduction of the concomitant CYP2D6 substrate. (7.2)

### Use in Specific Populations

Moderate or Severe Hepatic impairment: Avoid use. (8.6)

See 17 for PATIENT COUNSELING INFORMATION and FDA-approved patient labeling.

Revised: 12/2025

### Full Prescribing Information: Contents*

1 INDICATIONS AND USAGE *Sections or subsections omitted from the full prescribing information are not listed.
2 DOSAGE AND ADMINISTRATION
   2.1 Patient Selection
   2.2 Recommended Dosage
   2.3 Dosage Modification for Adverse Reactions
3 DOSAGE FORMS AND STRENGTHS
4 CONTRAINDICATIONS
5 WARNINGS AND PRECAUTIONS
   5.1 Myelodysplastic Syndrome/Acute Myeloid Leukemia
   5.2 Myelosuppression
   5.3 Hypokalemia, Fluid Retention, and Cardiovascular Adverse Reactions
   5.4 Hepatotoxicity
   5.5 Adrenocortical Insufficiency
   5.6 Hypoglycemia
   5.7 Increased Fractures and Mortality in Combination with Radium 223 Dichloride
   5.8 Posterior Reversible Encephalopathy Syndrome
   5.9 Embryo-Fetal Toxicity
6 ADVERSE REACTIONS
   6.1 Clinical Trial Experience
7 DRUG INTERACTIONS
   7.1 Effect of Other Drugs on AKEEGA
   7.2 Effects of AKEEGA on Other Drugs
8 USE IN SPECIFIC POPULATIONS
   8.1 Pregnancy
   8.2 Lactation
   8.3 Females and Males of Reproductive Potential
   8.4 Pediatric Use
   8.5 Geriatric Use
   8.6 Hepatic Impairment
   8.7 Renal Impairment
10 OVERDOSAGE
11 DESCRIPTION
12 CLINICAL PHARMACOLOGY
   12.1 Mechanism of Action
   12.2 Pharmacodynamics
   12.3 Pharmacokinetics
13 NONCLINICAL TOXICOLOGY
   13.1 Carcinogenesis, Mutagenesis, Impairment of Fertility
   13.2 Animal Toxicology and/or Pharmacology
14 CLINICAL STUDIES
   14.1 BRCA2-mutated Metastatic Castration-Sensitive Prostate Cancer (mCSPC)
   14.2 BRCA-mutated Metastatic Castration-Resistant Prostate Cancer (mCRPC)
16 HOW SUPPLIED/STORAGE AND HANDLING
17 PATIENT COUNSELING INFORMATION

### Full Prescribing Information

### 1 Indications and Usage

AKEEGA with prednisone is indicated for the treatment of adult patients with deleterious or suspected deleterious BRCA2-mutated (BRCA2m) metastatic castration-sensitive prostate cancer (mCSPC).

AKEEGA with prednisone is indicated for the treatment of adult patients with deleterious or suspected deleterious BRCA-mutated (BRCAm) metastatic castration-resistant prostate cancer (mCRPC).

Select patients for therapy based on an FDA-approved test for AKEEGA [see Dosage and Administration (2.1)].

### 2 Dosage and Administration

### 2.1 Patient Selection

Select patients for the treatment of mCSPC with AKEEGA based on the presence of a BRCA2 gene alteration [see Clinical Studies (14.1)].

Select patients for the treatment of mCRPC with AKEEGA based on the presence of a BRCA gene alteration [see Clinical Studies (14.2)].

Information on FDA-approved tests is available at: http://www.fda.gov/CompanionDiagnostics.

### 2.2 Recommended Dosage

BRCA2-Mutated (BRCA2m) Metastatic Castration-Sensitive Prostate Cancer (mCSPC)
The recommended dosage of AKEEGA is 200 mg niraparib/1,000 mg abiraterone acetate orally once daily in combination with 5 mg prednisone once daily until disease progression or unacceptable toxicity.

BRCA-Mutated (BRCAm) Metastatic Castration-Resistant Prostate Cancer (mCRPC)
The recommended dosage of AKEEGA is 200 mg niraparib/1,000 mg abiraterone acetate orally once daily in combination with 10 mg prednisone once daily until disease progression or unacceptable toxicity.

Patients receiving AKEEGA should also receive a gonadotropin-releasing hormone (GnRH) analog concurrently or should have had bilateral orchiectomy.

Take AKEEGA on an empty stomach at least one hour before or two hours after food. Swallow tablets whole with water. Do not break, crush, or chew tablets.

If a patient misses a dose, instruct patients to take the dose as soon as possible on the same day and resume their next dose at the normal schedule the following day.

### 2.3 Dosage Modification for Adverse Reactions

The recommended dosage modifications for AKEEGA are provided in Table 1.

Treatment with AKEEGA should not be reinitiated until the toxicity has resolved to Grade 1 or baseline. If the toxicity is attributed to one component of AKEEGA, the other component of AKEEGA may be continued as a single agent at the current dose until the adverse reaction resolves and AKEEGA can be resumed (see Table 1).

TABLE 1
Dosage Modifications for Adverse Reactions

| Adverse Reaction | Severity | Dosage Modification |
|---|---|---|
| Myelosuppression | Hemoglobin < 8 g/dL | Withhold AKEEGA and monitor |
| [see Warnings and | | blood counts weekly. |
| Precautions (5.2)] | | When hemoglobin returns to ≥9 |
| | | g/dL, resume at the reduced dose of |
| | | AKEEGA 100 mg/1,000 mg once |
| | | daily and monitor blood counts |
| | | weekly for 28 days and as clinically |
| | | indicated. |
| | | Permanently discontinue AKEEGA |
| | | if hemoglobin has not returned to |
| | | acceptable levels within 28 days of |
| | | the dose interruption period or if |
| | | the patient has already undergone |
| | | dose reduction to 100 mg/1,000 mg |
| | | once daily.^{a} |
| | Platelet count < 100, 000/mcL | First occurrence: |
| | | Withhold AKEEGA for a |
| | | maximum of 28 days and monitor |
| | | blood counts weekly until platelet |
| | | counts return to ≥100,000/mcL. |
| | | Resume AKEEGA at same or the |
| | | reduced dose of 100 mg/1,000 mg |
| | | once daily. |
| | | If platelet count is <75,000/mcL, |
| | | resume at the reduced dose of |
| | | AKEEGA 100 mg/1,000 mg once |
| | | daily. |
| | | Second occurrence: |
| | | Withhold AKEEGA for a |
| | | maximum of 28 days and monitor |
| | | blood counts weekly until platelet |
| | | counts return to ≥100,000/mcL. |
| | | Resume at the reduced dose of |
| | | AKEEGA 100 mg/1,000 mg once |
| | | daily. |
| | | Permanently discontinue AKEEGA |
| | | if the platelet count has not |
| | | returned to acceptable levels within |
| | | 28 days of the dose interruption |
| | | period or if the patient has already |
| | | undergone dose reduction to 100 |
| | | mg/1,000 mg once daily.^{a} |
| | Neutrophil < 1,000/mcL | Withhold AKEEGA and monitor |
| | | blood counts weekly. |
| | | When neutrophil counts return |
| | | to ≥1,500/mcL, resume at the |
| | | reduced dose of AKEEGA |
| | | 100 mg/1,000 mg once daily and |
| | | monitor blood counts weekly for |
| | | 28 days and as clinically indicated. |
| | | Permanently discontinue AKEEGA |
| | | if neutrophils have not returned to |
| | | acceptable levels within 28 days of |
| | | the dose interruption period or if |
| | | the patient has already undergone |
| | | dose reduction to 100 mg/1,000 mg |
| | | once daily.^{a} |
| | Hematologic adverse reaction | Consider platelet transfusion for |
| | requiring transfusion | patients with platelet count ≤ 10,000/mcL. |
| | | If there are other risk factors such as |
| | | coadministration of anticoagulation |
| | | or antiplatelet drugs, consider |
| | | interrupting these drugs and/or |
| | | transfusion at a higher platelet |
| | | count. |
| | | Resume at the reduced dose of |
| | | AKEEGA 100 mg/1,000 mg once |
| | | daily. |
| Hepatotoxicity | ALT and/or AST greater than | Withhold AKEEGA and closely |
| [see Warnings and | 5 × ULN or total bilirubin | monitor liver function. |
| Precautions (5.4)] | greater than 3 × ULN | Permanently discontinue AKEEGA |
| | | if: |
| | | ALT or AST ≥ 20 times the ULN |
| | | - OR- |
| | | ALT > 3 × ULN and total bilirubin > 2 × |
| | | ULN in the absence of biliary obstruction |
| | | or other causes responsible for the |
| | | concurrent elevation |
| | | -OR- |
| | | Hepatotoxicity recurs at the |
| | | reduced dose 100 mg/500 mg. |
| | | When AST and ALT resolves to |
| | | less ≤2.5 × ULN and total |
| | | bilirubin ≤ 1.5 × ULN, AKEEGA |
| | | may be resumed at the reduced |
| | | dose of 100 mg/500 mg once daily. |
| | | When resumed, monitor serum |
| | | transaminases every two weeks for |
| | | three months, monthly thereafter, |
| | | and as clinically indicated. |
| Other non-hematological | Grade 3 or 4^{b} | Withhold AKEEGA until |
| adverse reactions that persist | | resolution of adverse reaction or for |
| despite medical management | | a maximum of 28 days. |
| [see Warnings and | | If resolves in 28 days or less, |
| Precautions (5) and Adverse | | AKEEGA may be resumed at the |
| Reactions (6.1)] | | reduced dose. |
| | | Permanently discontinue AKEEGA |
| | | if adverse reaction(s) has not |
| | | resolved after 28 days or Grade 3 or |
| | | 4 adverse reaction reoccurs after |
| | | dose reduction. |

^{a}If myelodysplastic syndrome or acute myeloid leukemia (MDS/AML) is confirmed, discontinue AKEEGA [see Warnings and Precautions (5.1)].
^{b}Discontinue AKEEGA in patients who develop hypertensive crisis or other severe cardiovascular adverse reactions [see Warnings and Precautions (5.3)].

### 3 Dosage Forms and Strengths

Tablets
50 mg niraparib/500 mg abiraterone acetate: yellowish orange to yellowish brown, oval, film-coated tablets debossed with “N 50 A” on one side and plain on the other side.
100 mg niraparib/500 mg abiraterone acetate: orange, oval, film-coated tablets debossed with “N 100 A” on one side and plain on the other side.

### 4 Contraindications

None.

### 5 Warnings and Precautions

### 5.1 Myelodysplastic Syndrome/Acute Myeloid Leukemia

AKEEGA may cause myelodysplastic syndrome/acute myeloid leukemia (MDS/AML).

In the individual AMPLITUDE and MAGNITUDE studies, MDS or AML, including cases with fatal outcomes, were reported in 0.6% (2/347) and 0.5% (1/212) of patients treated with AKEEGA plus prednisone, respectively.

All patients in other tumor types treated with niraparib, a component of AKEEGA, who developed secondary MDS/cancer-therapy-related AML had received previous chemotherapy with platinum agents and/or other DNA-damaging agents, including radiotherapy.

For suspected MDS/AML or prolonged hematological toxicities, refer the patient to a hematologist for further evaluation. Discontinue AKEEGA if MDS/AML is confirmed.

### 5.2 Myelosuppression

AKEEGA may cause myelosuppression (anemia, thrombocytopenia, or neutropenia).

In AMPLITUDE, Grade 3-4 anemia, neutropenia, and thrombocytopenia were reported, respectively in 29%, 10%, and 4.9% of patients receiving AKEEGA. Overall, 25% of patients with anemia required a red blood cell transfusion, including 15% who required more than one transfusion. Discontinuation due to anemia occurred in 1.2% of patients.

In MAGNITUDE Cohort 1, Grade 3-4 anemia, thrombocytopenia, and neutropenia were reported, respectively in 28%, 8%, and 7% of patients receiving AKEEGA. Overall, 27% of patients with anemia required a red blood cell transfusion, including 19.5% who required more than one transfusion. Discontinuation due to anemia occurred in 3% of patients.

Monitor complete blood counts weekly during the first month of AKEEGA treatment, every two weeks for the next two months, monthly for the remainder of the first year and then every other month, and as clinically indicated. Do not start AKEEGA until patients have adequately recovered from hematologic toxicity caused by previous therapy. If hematologic toxicities do not resolve within 28 days following interruption, discontinue AKEEGA and refer the patient to a hematologist for further investigations, including bone marrow analysis and blood sample for cytogenetics [see Dosage and Administration (2.3)].

### 5.3 Hypokalemia, Fluid Retention, and Cardiovascular Adverse Reactions

AKEEGA may cause hypokalemia and fluid retention as a consequence of increased mineralocorticoid levels resulting from CYP17 inhibition [see Clinical Pharmacology (12.1)]. In post-marketing experience, QT prolongation and Torsades de Pointes have been observed in patients who develop hypokalemia while taking abiraterone acetate, a component of AKEEGA. Hypertension and hypertensive crisis have also been reported in patients treated with niraparib, a component of AKEEGA.

In AMPLITUDE, which used prednisone 5 mg daily in combination with AKEEGA, Grades 3-4 hypokalemia was detected in 9% of patients on the AKEEGA arm, and Grades 3-4 hypertension was observed in 30% of patients on the AKEEGA arm.

In MAGNITUDE Cohort 1, which used prednisone 10 mg daily in combination with AKEEGA, Grade 3-4 hypokalemia was detected in 2.7% of patients on the AKEEGA arm and Grade 3-4 hypertension was observed in 14% of patients on the AKEEGA arm.

Monitor patients for hypertension, hypokalemia, and fluid retention at least weekly for the first two months, then once a month. Closely monitor patients whose underlying medical conditions might be compromised by increases in blood pressure, hypokalemia, or fluid retention, such as those with heart failure, recent myocardial infarction, cardiovascular disease, or ventricular arrhythmia. Control hypertension and correct hypokalemia before and during treatment with AKEEGA. Discontinue AKEEGA in patients who develop hypertensive crisis or other severe cardiovascular adverse reactions.

The safety of AKEEGA in patients with New York Heart Association (NYHA) Class II to IV heart failure has not been established because these patients were excluded from AMPLITUDE and MAGNITUDE.

### 5.4 Hepatotoxicity

AKEEGA may cause hepatotoxicity.

Hepatotoxicity in patients receiving abiraterone acetate, a component of AKEEGA, has been reported in clinical trials. In post-marketing experience, there have been abiraterone acetate-associated severe hepatic toxicity, including fulminant hepatitis, acute liver failure, and deaths.

In AMPLITUDE, Grade 3-4 ALT or AST increases (at least 5× ULN) were reported in 1.9% and 1.3% of patients, respectively.

In MAGNITUDE Cohort 1, Grade 3-4 ALT or AST increases (at least 5× ULN) were reported in 1.8% and 0.9% of patients, respectively.

The safety of AKEEGA in patients with moderate or severe hepatic impairment has not been established as these patients were excluded from AMPLITUDE and MAGNITUDE.

Measure serum transaminases (ALT and AST) and bilirubin levels prior to starting treatment with AKEEGA, every two weeks for the first three months of treatment and monthly thereafter. Promptly measure serum total bilirubin, AST, and ALT if clinical symptoms or signs suggestive of hepatotoxicity develop. Elevations of AST, ALT, or bilirubin from the patient's baseline should prompt more frequent monitoring and may require dosage modifications [see Dosage and Administration (2.3)].

Permanently discontinue AKEEGA for patients who develop a concurrent elevation of ALT greater than 3× ULN and total bilirubin greater than 2× ULN in the absence of biliary obstruction or other causes responsible for the concurrent elevation, or in patients who develop ALT or AST ≥20× ULN at any time after receiving AKEEGA.

### 5.5 Adrenocortical Insufficiency

AKEEGA may cause adrenal insufficiency.

Adrenocortical insufficiency has been reported in clinical trials in patients receiving abiraterone acetate, a component of AKEEGA, in combination with prednisone, following interruption of daily steroids and/or with concurrent infection or stress. Monitor patients for symptoms and signs of adrenocortical insufficiency, particularly if patients are withdrawn from prednisone, have prednisone dose reductions, or experience unusual stress. Symptoms and signs of adrenocortical insufficiency may be masked by adverse reactions associated with mineralocorticoid excess seen in patients treated with abiraterone acetate. If clinically indicated, perform appropriate tests to confirm the diagnosis of adrenocortical insufficiency. Increased doses of corticosteroids may be indicated before, during, and after stressful situations.

### 5.6 Hypoglycemia

AKEEGA may cause hypoglycemia in patients being treated with other medications for diabetes.

Severe hypoglycemia has been reported when abiraterone acetate, a component of AKEEGA, was administered to patients receiving medications containing thiazolidinediones (including pioglitazone) or repaglinide [see Drug Interactions (7.2)].

Monitor blood glucose in patients with diabetes during and after discontinuation of treatment with AKEEGA. Assess if antidiabetic drug dosage needs to be adjusted to minimize the risk of hypoglycemia.

5.7 Increased Fractures and Mortality in Combination with Radium 223 Dichloride

AKEEGA with prednisone is not recommended for use in combination with Ra-223 dichloride outside of clinical trials.

The clinical efficacy and safety of concurrent initiation of abiraterone acetate plus prednisone/prednisolone and radium Ra 223 dichloride was assessed in a randomized, placebo-controlled multicenter study (ERA-223 trial) in 806 patients with asymptomatic or mildly symptomatic castration-resistant prostate cancer with bone metastases. The study was unblinded early based on an Independent Data Monitoring Committee recommendation.

At the primary analysis, increased incidences of fractures (29% vs 11%) and deaths (39% vs 36%) have been observed in patients who received abiraterone acetate plus prednisone/prednisolone in combination with radium Ra 223 dichloride compared to patients who received placebo in combination with abiraterone acetate plus prednisone.

It is recommended that subsequent treatment with Ra-223 not be initiated for at least five days after the last administration of AKEEGA, in combination with prednisone.

### 5.8 Posterior Reversible Encephalopathy Syndrome

AKEEGA may cause Posterior Reversible Encephalopathy Syndrome (PRES).

PRES has been observed in patients treated with niraparib as a single agent at higher than the recommended dose of niraparib included in AKEEGA.

Monitor all patients treated with AKEEGA for signs and symptoms of PRES. If PRES is suspected, promptly discontinue AKEEGA and administer appropriate treatment. The safety of reinitiating AKEEGA in patients previously experiencing PRES is not known.

### 5.9 Embryo-Fetal Toxicity

The safety and efficacy of AKEEGA have not been established in females. Based on animal reproductive studies and mechanism of action, AKEEGA can cause fetal harm and loss of pregnancy when administered to a pregnant female [see Clinical Pharmacology (12.1)].

Niraparib has the potential to cause teratogenicity and/or embryo-fetal death since niraparib is genotoxic and targets actively dividing cells in animals and patients (e.g., bone marrow) [see Warnings and Precautions (5.2) and Nonclinical Toxicology (13.1)].

In animal reproduction studies, oral administration of abiraterone acetate to pregnant rats during organogenesis caused adverse developmental effects at maternal exposures approximately ≥0.03 times the human exposure (AUC) at the recommended dose.

Advise males with female partners of reproductive potential to use effective contraception during treatment and for 4 months after the last dose of AKEEGA [see Use in Specific Populations (8.1, 8.3)]. Females who are or may become pregnant should handle AKEEGA with protection, e.g., gloves [see How Supplied/Storage and Handling (16)].

### 6 Adverse Reactions

The following adverse reactions are discussed elsewhere in the labeling:
Myelodysplastic syndrome/acute myeloid leukemia [see Warnings and Precautions (5.1)]
Myelosuppression [see Warnings and Precautions (5.2)]
Hypokalemia, fluid retention, and cardiovascular adverse reactions [see Warnings and Precautions (5.3)]
Hepatotoxicity [see Warnings and Precautions (5.4)]
Adrenocortical insufficiency [see Warnings and Precautions (5.5)]
Hypoglycemia [see Warnings and Precautions (5.6)]
Increased fractures and mortality in combination with Radium 223 Dichloride [see Warnings and Precautions (5.7)]
Posterior reversible encephalopathy syndrome [see Warnings and Precautions (5.8)]

### 6.1 Clinical Trial Experience

Because clinical trials are conducted under widely varying conditions, adverse reaction rates observed in the clinical trials of a drug cannot be directly compared to rates in the clinical trials of another drug and may not reflect the rates observed in practice.

The safety population described in the WARNINGS and PRECAUTIONS reflect exposure to AKEEGA (niraparib 200 mg and abiraterone acetate 1,000 mg) in BRCA2m patients (N=162) in the AMPLITUDE study and in BRCAm patients in Cohort 1 (N=113) in the MAGNITUDE study unless otherwise specified.

BRCA2-mutated Metastatic Castration-Sensitive Prostate Cancer (mCSPC)

The safety of AKEEGA in patients with BRCA2m mCSPC was evaluated in AMPLITUDE [see Clinical Studies (14.1)]. Patients were randomized to receive either AKEEGA (niraparib 200 mg and abiraterone acetate 1,000 mg once daily) (n=162), or placebo and abiraterone acetate (n=161) until unacceptable toxicity or progression. Patients in both arms also received prednisone 5 mg daily. The median duration of exposure for AKEEGA was 26 months (range: 0 to 48 months).

Serious adverse reactions occurred in 36% of patients who received AKEEGA. Serious adverse reactions reported in >2% of patients included anemia (4.9%), and pneumonia (3.7%). Fatal adverse reactions occurred in 4.9% of patients who received AKEEGA, including sudden death (1.9%), COVID-19 pneumonia (1.2%), pneumocystis jirovecii pneumonia (0.6%), pneumonia (0.6%), and cardio-respiratory arrest (0.6%).

Permanent discontinuation of any component of AKEEGA due to an adverse reaction occurred in 13% of patients.

Dosage interruptions of any component of AKEEGA due to an adverse reaction occurred in 67% of patients. Adverse reactions which required dosage interruption in >2% of patients included anemia (30%), COVID-19 (10%), hypertension (9%), neutropenia (8%), thrombocytopenia (8%), hypokalemia (7%), vomiting (4.9%), fatigue (4.3%), diarrhea (2.5%), and pneumonia (2.5%).

Dose reductions of any component of AKEEGA due to an adverse reaction occurred in 25% of patients. Adverse reactions which required dose reductions in >2% of patients included anemia (17%).

The most common adverse reactions (>20%), including laboratory abnormalities, in patients who received AKEEGA were decreased hemoglobin, decreased lymphocyte count, hypertension, decreased neutrophil count, musculoskeletal pain, decreased platelet count, constipation, fatigue, decreased potassium, increase creatinine, nausea, increased alkaline phosphate, increased aspartate aminotransferase, respiratory tract infection, arrhythmia, increased blood bilirubin, and fluid retention/edema.

TABLE 2
Adverse Reactions (>20%) in Patients with BRCA2m
mCSPC Who Received AKEEGA (with a Difference of ≥5%
Compared to Placebo) in AMPLITUDE

| | AKEEGA | | Placebo with Abiraterone | |
|---|---|---|---|---|
| | (N = 162) | | Acetate (N = 161) | |
| Adverse | All Grades | Grade 3 or 4 | All Grades | Grade 3 or 4 |
| Reaction | % | % | % | % |

Vascular disorders

| | | | | |
|---|---|---|---|---|
| Hypertension^{a} | 51 | 31 | 36 | 19 |

Musculoskeletal and connective tissue disorders

| | | | | |
|---|---|---|---|---|
| Musculoskeletal | 45 | 6 | 58 | 4.3 |
| pain^{a} | | | | |

Gastrointestinal disorders

| | | | | |
|---|---|---|---|---|
| Constipation | 41 | 0 | 17 | 0.6 |
| Nausea | 30 | 0 | 17 | 0 |

General disorders and administration

| | | | | |
|---|---|---|---|---|
| Fatigue^{a} | 39 | 4.3 | 29 | 3.1 |

Respiratory, thoracic and mediastinal disorders

| | | | | |
|---|---|---|---|---|
| Respiratory | 23 | 0.6 | 13 | 0.6 |
| Tract Infection^{a} | | | | |

Cardiac disorders

| | | | | |
|---|---|---|---|---|
| Arrhythmia^{a} | 23 | 3.7 | 9 | 2.5 |

^{a}Grouped terms including multiple similar terms

Clinically relevant adverse reactions that occurred in ≤20% of patients receiving AKEEGA plus prednisone were hot flush (18%), vomiting (17%), dizziness (17%), abdominal pain (15%), weight decreased (14%), diarrhea (14%), decreased appetite (12%), headache (12%), hemorrhage (12%), dyspnea (10%), urinary tract infection (8%), pneumonia (7%), osteoporosis (4.9%), rash (3.7%), cardiac failure (3.1%), ischemic heart disease (4.9%), acute kidney injury (2.5%), pulmonary embolism (2.5%), and urosepsis (0.6%).

The most common select laboratory abnormalities (>20%) that worsened from baseline in patients who received AKEEGA are in Table 3.

TABLE 3
Select Laboratory Abnormalities (>20%) That
Worsened from Baseline in Patients with BRCA2m
mCSPC Who Received AKEEGA in AMPLITUDE

| | AKEEGA¹ | | Placebo with Abiraterone | |
|---|---|---|---|---|
| | (N = 162) | | Acetate¹ (N = 161) | |
| Laboratory | All Grades | Grade 3 or 4 | All Grades | Grade 3 or 4 |
| Abnormality | (%) | (%) | (%) | (%) |

Hematology

| | | | | |
|---|---|---|---|---|
| Decreased | 74 | 29 | 53 | 1.9 |
| Hemoglobin | | | | |
| Decreased | 59 | 20 | 37 | 13 |
| Lymphocyte Count | | | | |
| Decreased | 49 | 10 | 19 | 3.1 |
| Neutrophil Count | | | | |
| Decreased Platelet | 41 | 4.9 | 23 | 0.6 |
| Count | | | | |

Chemistry

| | | | | |
|---|---|---|---|---|
| Decreased | 38 | 9 | 29 | 10 |
| Potassium | | | | |
| Increased Creatinine | 30 | 1.3 | 16 | 2.5 |
| Increased Alkaline | 28 | 0.6 | 24 | 3.1 |
| Phosphatase | | | | |
| Increased Aspartate | 24 | 1.3 | 33 | 2.5 |
| Aminotransferase | | | | |
| Increased Blood | 22 | 0 | 11 | 0 |
| Bilirubin | | | | |

¹The denominator used to calculate the rate varied from 160 to 161 for placebo with abiraterone acetate plus prednisone and 159 to 162 for AKEEGA with prednisone based on the number of patients with a baseline value and at least one post-treatment value.

### BRCA-mutated Metastatic Castration-Resistant Prostate Cancer

The safety of AKEEGA in patients with BRCAm mCRPC was evaluated in Cohort 1 of MAGNITUDE [see Clinical Studies (14.2)]. Patients were randomized to receive either AKEEGA (niraparib 200 mg and abiraterone acetate 1,000 mg once daily) (n=113), or placebo and abiraterone acetate (n=112) until unacceptable toxicity or progression. Patients in both arms also received prednisone 10 mg daily. The median duration of exposure for AKEEGA was 18 months (range: 0 to 37 months).

Serious adverse reactions occurred in 41% of patients who received AKEEGA. Serious adverse reactions reported in >2% of patients included COVID-19 (7%), anemia (4.4%), pneumonia (3.5%), and hemorrhage (3.5%). Fatal adverse reactions occurred in 9% of patients who received AKEEGA, including COVID-19 (5%), cardiopulmonary arrest (1%), dyspnea (1%), pneumonia (1%), and septic shock (1%).

Permanent discontinuation of any component of AKEEGA due to an adverse reaction occurred in 15% of patients. Adverse reactions which resulted in permanent discontinuation of AKEEGA in >2% of patients included COVID-19 (4.4%), anemia (2.7%), asthenia (2.7%), and vomiting (2.7%).

Dosage interruptions of any component of AKEEGA due to an adverse reaction occurred in 50% of patients. Adverse reactions which required dosage interruption in >2% of patients included anemia (23%), thrombocytopenia (12%), neutropenia (7%), COVID-19 (6%), fatigue (3.5%), asthenia (3.5%), nausea (3.5%), pneumonia (2.7%), hematuria (2.7%), and vomiting (2.7%).

Dose reductions of any component of AKEEGA due to an adverse reaction occurred in 28% of patients. Adverse reactions which required dose reductions in >2% of patients included anemia (12%), thrombocytopenia (4.4%), and fatigue (2.7%).

The most common adverse reactions (>20%), including laboratory abnormalities, in patients who received AKEEGA were hemoglobin decreased, lymphocyte decreased, musculoskeletal pain, fatigue, platelets decreased, constipation, alkaline phosphatase increased, hypertension, nausea, neutrophils decreased, creatinine increased, potassium increased, potassium decreased, and aspartate aminotransferase increased.

Tables 4 and 5 summarize adverse reactions and laboratory abnormalities for patients with BRCAm mCRPC in MAGNITUDE, respectively.

TABLE 4
Adverse Reactions (>10%) in Patients with
BRCAm mCRPC Who Received AKEEGA in MAGNITUDE

| | AKEEGA | | Placebo with Abiraterone | |
|---|---|---|---|---|
| | (N = 113) | | Acetate (N = 112) | |
| | All | Grade | All | Grade |
| | Grades | 3 or 4 | Grades | 3 or 4 |
| Adverse Reaction | % | % | % | % |

Musculoskeletal and connective tissue disorders

| | | | | |
|---|---|---|---|---|
| Musculoskeletal pain^{a} | 44 | 4 | 42 | 5 |

General disorders and administration site conditions

| | | | | |
|---|---|---|---|---|
| Fatigue^{a} | 43 | 5 | 30 | 4 |
| Edema^{a} | 17 | 0 | 9 | 0 |
| Pyrexia^{a} | 10 | 2 | 6 | 0 |

Gastrointestinal disorders

| | | | | |
|---|---|---|---|---|
| Constipation | 34 | 1 | 20 | 0 |
| Vomiting | 15 | 0 | 7 | 1 |
| Nausea | 33 | 1 | 21 | 0 |
| Abdominal pain^{a} | 12 | 2 | 12 | 1 |

Vascular disorders

| | | | | |
|---|---|---|---|---|
| Hypertension^{a} | 33 | 14 | 27 | 17 |
| Hemorrhage^{a} | 12 | 2 | 8 | 1 |

Respiratory, thoracic and mediastinal disorders

| | | | | |
|---|---|---|---|---|
| Dyspnea^{a} | 15 | 1 | 8 | 3 |
| Cough^{a} | 12 | 0 | 6 | 0 |

Metabolism and nutrition disorders

| | | | | |
|---|---|---|---|---|
| Decreased appetite | 15 | 2 | 8 | 0 |

Nervous system disorders

| | | | | |
|---|---|---|---|---|
| Dizziness^{a} | 14 | 0 | 10 | 0 |
| Headache | 12 | 1 | 9 | 0 |

Infections and infestations

| | | | | |
|---|---|---|---|---|
| COVID-19^{a} | 13 | 7 | 9 | 4 |
| Urinary tract infection^{a} | 12 | 3 | 9 | 1 |

Psychiatric disorders

| | | | | |
|---|---|---|---|---|
| Insomnia | 12 | 0 | 4 | 0 |

Investigations

| | | | | |
|---|---|---|---|---|
| Weight decreased | 10 | 1 | 4 | 1 |

Cardiac disorders

| | | | | |
|---|---|---|---|---|
| Arrhythmia^{a} | 10 | 2 | 4 | 1 |

Injury, poisoning and procedural complications

| | | | | |
|---|---|---|---|---|
| Fall | 10 | 1 | 13 | 4 |

^{a}Grouped terms including multiple similar terms.

Clinically relevant adverse events that occurred in <10% of patients receiving AKEEGA plus prednisone were rash (7%), alanine aminotransferase increased (5%), aspartate aminotransferase increased (5%), cerebrovascular accident (4.4%), pulmonary embolism (2.7%), deep vein thrombosis (2.7%), and acute kidney injury (2.7%).

TABLE 5
Select Laboratory Abnormalities (>20%) That Worsened from Baseline
in Patients with BRCAm mCRPC Who Received AKEEGA in MAGNITUDE

| | AKEEGA¹ | | Placebo with Abiraterone | |
|---|---|---|---|---|
| | (N = 113) | | Acetate¹ (N = 112) | |
| | All Grades | Grade 3 or 4 | All Grades | Grade 3 or 4 |
| Laboratory Abnormality | (%) | (%) | (%) | (%) |

Hematology

| | | | | |
|---|---|---|---|---|
| Hemoglobin decreased | 67 | 26 | 53 | 7 |
| Lymphocyte decreased | 55 | 22 | 32 | 13 |
| Platelets decreased | 37 | 8 | 22 | 1.8 |
| Neutrophils decreased | 32 | 7 | 16 | 2.7 |

Chemistry

| | | | | |
|---|---|---|---|---|
| Alkaline Phosphatase increased | 34 | 1.8 | 29 | 1.8 |
| Creatinine increased | 30 | 0 | 13 | 1.8 |
| Potassium increased | 25 | 0.9 | 21 | 3.6 |
| Potassium decreased | 20 | 5 | 20 | 5 |
| Aspartate Aminotransferase | 20 | 1.8 | 25 | 2.7 |
| increased | | | | |

¹The denominator used to calculate the rate varied from 111 to 112 for placebo with abiraterone acetate plus prednisone and 113 for AKEEGA with prednisone based on the number of patients with a baseline value and at least one post-treatment value.

### Other Clinical Trial Experience

The following adverse reactions have been reported with the individual components of AKEEGA but were not observed in AMPLITUDE or MAGNITUDE Cohort 1: myopathy, rhabdomyolysis, adrenal insufficiency, allergic alveolitis, febrile neutropenia, anaphylactic reaction, posterior reversible encephalopathy (PRES), and hypertensive crisis.

### 7 Drug Interactions

### 7.1 Effect of Other Drugs on AKEEGA

### Effect of CYP3A4 Inducers

Avoid coadministration with strong CYP3A4 inducers [see Clinical Pharmacology (12.3)].

Abiraterone is a substrate of CYP3A4. Strong CYP3A4 inducers may decrease abiraterone concentrations [see Clinical Pharmacology (12.3)], which may reduce the effectiveness of abiraterone.

### 7.2 Effects of AKEEGA on Other Drugs

### CYP2D6 Substrates

Avoid coadministration unless otherwise recommended in the Prescribing Information for CYP2D6 substrates for which minimal changes in concentration may lead to serious toxicities. If alternative treatments cannot be used, consider a dose reduction of the concomitant CYP2D6 substrate drug.

Abiraterone is a CYP2D6 moderate inhibitor. AKEEGA increases the concentration of CYP2D6 substrates [see Clinical Pharmacology (12.3)], which may increase the risk of adverse reactions related to these substrates.

### CYP2C8 Substrates

Monitor patients for signs of toxicity related to a CYP2C8 substrate for which a minimal change in plasma concentration may lead to serious or life-threatening adverse reactions.

Abiraterone is a CYP2C8 inhibitor. AKEEGA increases the concentration of CYP2C8 substrates [see Clinical Pharmacology (12.3)], which may increase the risk of adverse reactions related to these substrates.

### 8 Use in Specific Populations

### 8.1 Pregnancy

### Risk Summary

The safety and efficacy of AKEEGA have not been established in females. Based on findings from animal studies and mechanism of action [see Clinical Pharmacology (12.1)], AKEEGA can cause fetal harm and potential loss of pregnancy.

There are no human data on the use of AKEEGA in pregnant women.

Niraparib has the potential to cause teratogenicity and/or embryo-fetal death since niraparib is genotoxic and targets actively dividing cells in animals and patients (e.g., bone marrow) [see Warnings and Precautions (5.2) and Nonclinical Toxicology (13.1)]. Due to the potential risk to a fetus based on its mechanism of action, animal developmental and reproductive toxicology studies were not conducted with niraparib.

In animal reproduction studies, oral administration of abiraterone acetate to pregnant rats during organogenesis caused adverse developmental effects at maternal exposures approximately ≥0.03 times the human exposure (AUC) at the recommended dose (see Data).

### Data

### Animal Data

### Niraparib

Niraparib is genotoxic and targets actively dividing cells. Animal developmental and reproductive toxicology studies were not conducted with niraparib.

### Abiraterone Acetate

In an embryo-fetal developmental toxicity study in rats, abiraterone acetate caused developmental toxicity when administered at oral doses of 10, 30 or 100 mg/kg/day throughout the period of organogenesis (gestational days 6-17). Findings included embryo-fetal lethality (increased post implantation loss and resorptions and decreased number of live fetuses), fetal developmental delay (skeletal effects) and urogenital effects (bilateral ureter dilation) at doses ³10 mg/kg/day, decreased fetal ano-genital distance at 330 mg/kg/day, and decreased fetal body weight at 100 mg/kg/day. Doses ³10 mg/kg/day caused maternal toxicity. The doses tested in rats resulted in systemic exposures (AUC) approximately 0.03, 0.1 and 0.3 times, respectively, the AUC in patients receiving 1,000 mg daily of abiraterone acetate.

### 8.2 Lactation

### Risk Summary

The safety and efficacy of AKEEGA have not been established in females. There is no information available on the presence of niraparib or abiraterone in human milk, or on the effects on the breastfed child or milk production.

### 8.3 Females and Males of Reproductive Potential

### Contraception

### Males

Based on findings in animal reproduction studies and its mechanism of action, advise males with female partners of reproductive potential to use effective contraception during treatment and for 4 months after the last dose of AKEEGA [see Use in Specific Populations (8.1)].

### Infertility

Based on animal studies, AKEEGA may impair fertility in males of reproductive potential [see Nonclinical Toxicology (13.1)].

### 8.4 Pediatric Use

Safety and effectiveness of AKEEGA in pediatric patients have not been established.

### 8.5 Geriatric Use

Of the 162 patients with BRCA2 gene alteration(s) who received AKEEGA in AMPLITUDE, 40% of patients were less than 65 years, 36% of patients were 65 years to 74 years, and 23% were 75 years and over.

Of the 113 patients with BRCA gene alteration(s) who received AKEEGA in MAGNITUDE, 34.5% of patients were less than 65 years, 38.9% of patients were 65 years to 74 years, and 26.5% were 75 years and over.

No overall differences in effectiveness were observed between patients 65 years of age or older and younger patients in AMPLITUDE or MAGNITUDE. Patients 75 years of age or older who received AKEEGA experienced a higher incidence of fatal adverse reactions than younger patients. The incidence of fatal adverse reactions was 4.3% in patients younger than 75 and 13% in patients 75 or older.

### 8.6 Hepatic Impairment

Avoid use of AKEEGA in patients with moderate or severe hepatic impairment [see Warnings and Precautions (5.4) and Clinical Pharmacology (12.3)].

No dosage modification is necessary for patients with mild hepatic impairment.

### 8.7 Renal Impairment

Monitor patients with severe renal impairment for increased adverse reactions and modify dosage as recommended for adverse reactions [see Clinical Pharmacology (12.3)].

No dosage modification is recommended for patients with mild to moderate renal impairment.

### 10 Overdosage

In the event of an overdose, administration of AKEEGA should be stopped and general supportive measures undertaken, including monitoring for arrhythmias and cardiac failure and assessing liver function.

There is no specific treatment in the event of AKEEGA overdose.

### 11 Description

AKEEGA® (niraparib and abiraterone acetate) tablets contain niraparib tosylate (as the monohydrate) and abiraterone acetate.

### Niraparib

Niraparib is a poly (ADP-ribose) polymerase (PARP) inhibitor. The chemical name for niraparib tosylate monohydrate is 2-{4-[(3S)-piperidin-3-yl]phenyl}-2H-indazole 7-carboxamide 4-methylbenzenesulfonate hydrate (1:1:1). The molecular formula is C₂₆H₃₀N₄O₅S and it has a molecular weight of 510.61 g/mol. The molecular structure is shown below:

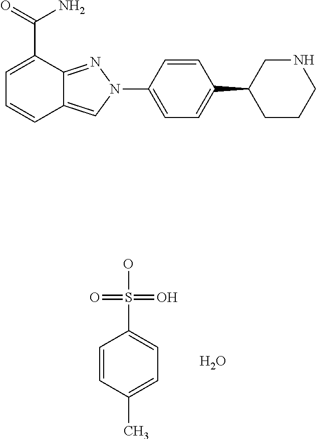 [SMILES: NC(=O)c1cccc2cn(-c3ccc([C@@H]4CCCNC4)cc3)nc12 ; Cc1ccc(S(=O)(=O)O)cc1 ; O]

Niraparib tosylate monohydrate is a white to off-white, non-hygroscopic crystalline solid. Niraparib tosylate monohydrate is highly soluble in aqueous media over the pH range 1.2 to 6.8 (1.65-1.77 mg/mL determined at 37±1° C.).

### Abiraterone Acetate

Abiraterone acetate is the acetyl ester of abiraterone. Abiraterone is an inhibitor of CYP17 (17α-hydroxylase/C17,20-lyase). Its molecular formula is C₂₆H₃₃NO₂ and it has a molecular weight of 391.55 g/mol. Abiraterone acetate is designated chemically as (3B)-17-(3-pyridinyl) androsta-5,16-dien-3-yl acetate and its structure is:

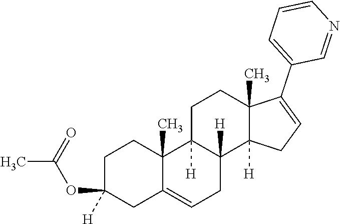

Abiraterone acetate is a white to off-white, non-hygroscopic, crystalline powder. Abiraterone acetate is a lipophilic compound with an octanol-water partition coefficient of 5.12 (Log P) and is practically insoluble in water. The pKa of the aromatic nitrogen is 5.19.

AKEEGA tablets are supplied as 50 mg/500 mg niraparib/abiraterone acetate and 100 mg/500 mg niraparib/abiraterone acetate film-coated tablets for oral administration.

Each AKEEGA tablet (50 mg/500 mg) contains 50 mg of niraparib (equivalent to 76.9 mg niraparib tosylate) and 500 mg of abiraterone acetate.

Each AKEEGA tablet (100 mg/500 mg) contains 100 mg of niraparib (equivalent to 153.7 mg niraparib tosylate) and 500 mg of abiraterone acetate.

AKEEGA tablet core contains the following inactive ingredients: colloidal anhydrous silica, crospovidone, hypromellose, lactose monohydrate, magnesium stearate, silicified microcrystalline cellulose, sodium lauryl sulfate.
The 50 mg/500 mg tablets are finished with film-coating comprising the following inactive ingredients: iron oxide black, iron oxide red, iron oxide yellow, sodium lauryl sulphate, glycerol monocaprylocaprate, polyvinyl alcohol, talc, and titanium dioxide.
The 100 mg/500 mg tablets are finished with film-coating comprising the following inactive ingredients: iron oxide red, iron oxide yellow, sodium lauryl sulphate, glycerol monocaprylocaprate, polyvinyl alcohol, talc, and titanium dioxide.

### 12 Clinical Pharmacology

### 12.1 Mechanism of Action

Niraparib is an inhibitor of PARP enzymes, including PARP-1 and PARP-2, that play a role in DNA repair. In vitro studies have shown that niraparib-induced cytotoxicity may involve inhibition of PARP enzymatic activity and increased formation of PARP-DNA complexes resulting in DNA damage, apoptosis, and cell death. Increased niraparib-induced cytotoxicity was observed in tumor cell lines with or without deficiencies in BRCA1/2. Niraparib decreased tumor growth in mouse xenograft models of human cancer cell lines with deficiencies in BRCA1/2 and in human patient-derived xenograft tumor models with homologous recombination deficiency (HRD) that had either mutated or wild-type BRCA1/2.

Abiraterone acetate is converted in vivo to abiraterone, an androgen biosynthesis inhibitor, that inhibits 17 α-hydroxylase/C17,20-lyase (CYP17). This enzyme is expressed in testicular, adrenal, and prostatic tumor tissues and is required for androgen biosynthesis.

CYP17 catalyzes two sequential reactions: 1) the conversion of pregnenolone and progesterone to their 17α-hydroxy derivatives by 17a-hydroxylase activity and 2) the subsequent formation of dehydroepiandrosterone (DHEA) and androstenedione, respectively, by C17, 20 lyase activity. DHEA and androstenedione are androgens and are precursors of testosterone. Inhibition of CYP17 by abiraterone can also result in increased mineralocorticoid production by the adrenals [see Warnings and Precautions (5.9)].

Androgen sensitive prostatic carcinoma responds to treatment that decreases androgen levels. Androgen deprivation therapies, such as treatment with GnRH agonists or orchiectomy, decrease androgen production in the testes but do not affect androgen production by the adrenals or in the tumor.

Abiraterone decreased serum testosterone and other androgens in patients in the placebo-controlled clinical trial. It is not necessary to monitor the effect of abiraterone on serum testosterone levels.

Changes in serum prostate specific antigen (PSA) levels may be observed but have not been shown to correlate with clinical benefit in individual patients.

In mouse xenograft models of prostate cancer, the combination of niraparib and abiraterone acetate increased anti-tumor activity when compared to either drug alone.

### 12.2 Pharmacodynamics

The exposure-response relationship and time-course of pharmacodynamic response for the safety and effectiveness of AKEEGA have not been fully characterized.

### Hypertension and Cardiovascular Effects

Niraparib has the potential to cause effects on pulse rate and blood pressure in patients, which may be related to pharmacological inhibition of the dopamine transporter (DAT), norepinephrine transporter (NET), and serotonin transporter (SERT) [see Nonclinical Toxicology (13.2)].

Niraparib increased mean pulse rate by 22.4 to 24.1 beats/min, mean systolic blood pressure by 24.5 mmHg, and mean diastolic pressure by 16.5 mmHg relative to 14.0 to 15.8 beats per min, 18.3 to 19.6 mmHg, and 11.6 mmHg in the placebo arm.

### Cardiac Electrophysiology

No large (>20 ms) increases in the mean QTc interval were detected following the treatment of niraparib 300 mg once daily or 1,000 mg of abiraterone acetate once daily.

12.3 Pharmacokinetics

### Niraparib

Following the administration of AKEEGA, the mean (coefficient of variation [CV %]) C_{max,ss} was 831 ng/ml (32%) and AUC_{0-24h,ss} was 13,616 ng·h/mL (36%). The accumulation ratio following daily administration of AKEEGA was 3.5-, and 2.6-fold for niraparib AUC₀₋₂₄ₕ and Cₘₐₓ.

Niraparib exhibits dose proportional increase in Cₘₐₓ and AUC in the dose range of 30 mg (0.15 times the recommended dosage) to 400 mg (2 times the recommended dosage).

### Abiraterone Acetate

Following the administration of AKEEGA, the mean (CV %) C_{max,ss} was 151 ng/ml (59%) and AUC_{0-24h,ss} was 707 ng·h/mL (59%) for abiraterone. The accumulation ratio following daily administration of AKEEGA was 2-, and 1.8-fold for abiraterone AUC₀₋₂₄ₕ and Cₘₐₓ.

No major deviation from dose proportionality was observed for abiraterone acetate in the dose range of 250 mg (0.25 times the recommended dosage) to 1,000 mg (the recommended dosage).

### Absorption

### Niraparib

The median Tₘₐₓ was 3 hours after dosing. The absolute bioavailability of niraparib is approximately 73%.

### Abiraterone Acetate

The median Tₘₐₓ of abiraterone was 1.5 hours after dosing.

Administration of abiraterone acetate with food, compared with administration in a fasted state, results in up to a 10-fold (AUC) and up to a 17-fold (Cₘₐₓ) increase in mean systemic exposure of abiraterone, depending on the fat content of the meal. Given the normal variation in the content and composition of meals, taking abiraterone acetate with meals has the potential to result in increased and highly variable exposures.

### Distribution

### Niraparib

The apparent volume of distribution of niraparib was 1,117 L. Niraparib is 83% bound to human plasma proteins.

### Abiraterone Acetate

The apparent volume of distribution of abiraterone was 25,774 L. Abiraterone is highly bound (>99%) to the human plasma proteins, albumin and alpha-1 acid glycoprotein.

### Elimination

### Niraparib

The mean t_{1/2} of niraparib when given in combination was approximately 62 hours (CV %: 42%) and apparent CL/F was 16.7 L/h (CV %: 27%).

### Abiraterone Acetate

The mean t_{1/2} of abiraterone when given in combination was approximately 20 hours (CV %: 7.8%) and apparent CL/F was 1673 L/h (CV %: 24%).

### Metabolism

### Niraparib

Niraparib is metabolized by carboxylesterases.

### Abiraterone Acetate

Abiraterone acetate is rapidly converted in vivo to abiraterone. CYP3A4 and SULT2A1 are the enzymes involved in the metabolism of abiraterone.

### Excretion

### Niraparib

About 48% (33% to 60%) of the radiolabeled dose was recovered in urine and 39% (28% to 47%) in feces. Unchanged niraparib accounted for 11% and 19% of the administered dose recovered in urine and feces, respectively.

### Abiraterone Acetate

Approximately 88% of the radiolabeled dose is recovered in feces and 5% in urine. Unchanged abiraterone acetate and abiraterone accounted for 55% and 22% of the administered dose recovered in the feces, respectively.

### Specific Populations

No clinically significant effects on the PK of niraparib and abiraterone were observed based on body weight (43.3-165 kg for niraparib and 46-165 kg for abiraterone), age (45-90 years for niraparib and 43-90 years for abiraterone), race/ethnicity (White, Asian, and Hispanic) and mild to moderate renal impairment (CLcr: 30-90 mL/min). Severe renal impairment (CLcr: 15-30 mL/min) has not been studied.

### Hepatic Impairment

### Niraparib

Mild hepatic impairment did not affect the exposure of niraparib. Moderate hepatic impairment (Total bilirubin >1.5 to 3× ULN and any aspartate aminotransferase value) increased niraparib AUC by 56% compared to that of patients with normal hepatic function.

### Abiraterone Acetate

Mild (Child-Pugh score of 5 to 6; Child-Turcotte-Pugh Class A) hepatic impairment increased abiraterone (AUC) by 1.1-fold and moderate (Child-Pugh score of 7 to 9; Child-Turcotte-Pugh Class B) hepatic impairment increased abiraterone (AUC) by 3.6-fold compared to subjects with normal hepatic function.

Severe (Child-Pugh score of 10 to 15; Child-Turcotte-Pugh Class C) hepatic impairment increased abiraterone AUC by 7-fold and the fraction of free drug increased by 2-fold in subjects compared to subjects with normal hepatic function.

### Drug Interactions Studies

### Niraparib

### In Vitro Studies

Inhibition of Cytochrome P450 (CYP) Enzymes: Niraparib is not an inhibitor of CYP1A2, CYP2B6, CYP2C8, CYP2C9, CYP2C19, CYP2D6, and CYP3A4.
Induction of CYP Enzymes: Niraparib is not a CYP3A4 inducer. Niraparib induces CYP1A2 in vitro.
Inhibition of Uridine 5′-Diphospho-Glucuronosyltransferases (UGTs): Niraparib did not inhibit UGT1A1, UGT1A4, UGT1A9, and UGT2B7.
Inhibition of Transporter Systems: Niraparib inhibits BCRP, but does not inhibit P-gp, BSEP, or MRP2.
Niraparib inhibits MATE 1 and 2.
Substrate of Transporter Systems: Niraparib is a substrate of P-gp and BCRP. Niraparib is not a substrate of BSEP, MRP2, or MATE1 or 2.

### Abiraterone Acetate

### Clinical Studies.

Effect of Strong CYP3A4 Inducers on Abiraterone: Coadministration of rifampin (strong CYP3A4 inhibitor) decreased abiraterone mean AUC by 55%.
Effect of Strong CYP3A4 Inhibitors on Abiraterone: Coadministration of ketoconazole (strong CYP3A4 inhibitor) had no clinically meaningful effect on the pharmacokinetics of abiraterone.
Effect of Abiraterone Acetate on CYP2D6 Substrates: The Cₘₐₓ and AUC of dextromethorphan (CYP2D6 substrate) were increased 2.8- and 2.9-fold, respectively when dextromethorphan 30 mg was given with abiraterone acetate 1,000 mg daily (plus prednisone). The AUC for dextrorphan, the active metabolite of dextromethorphan, increased approximately 1.3-fold.
Effect of Abiraterone Acetate on CYP1A2 Substrates: When abiraterone acetate (plus prednisone) was given with a single dose of 100 mg theophylline (CYP1A2 substrate), no increase in systemic exposure of theophylline was observed.
Effect of Abiraterone Acetate on CYP2C8 Substrates: The AUC of pioglitazone (CYP2C8 substrate) was increased by 46% when pioglitazone was given to healthy subjects with a single dose of abiraterone acetate.
   In vitro Studies
Cytochrome P450 (CYP) Enzymes: Abiraterone is a substrate of CYP3A4 and has the potential to inhibit CYP1A2, CYP2D6, CYP2C8 and to a lesser extent CYP2C9, CYP2C19 and CYP3A4/5.
Transporter Systems: Abiraterone acetate and abiraterone are not substrates of P-gp. Abiraterone acetate is an inhibitor of P-gp. Abiraterone and its major metabolites were inhibitors of OATP1B1.

### 13 Nonclinical Toxicology

### 13.1 Carcinogenesis, Mutagenesis, Impairment of Fertility

### Niraparib

Carcinogenicity studies have not been conducted with niraparib.

Niraparib was clastogenic in an in vitro mammalian chromosomal aberration assay and in an in vivo rat bone marrow micronucleus assay. This clastogenicity is consistent with genomic instability resulting from the primary pharmacology of niraparib and indicates potential for genotoxicity in humans. Niraparib was not mutagenic in a bacterial reverse mutation assay (Ames) test.

Fertility studies in animals have not been conducted with niraparib. In repeat-dose oral toxicity studies, niraparib was administered daily for up to 3 months' duration in rats and dogs. Reduced sperm, spermatids, and germ cells in epididymides and testes were observed at doses ≥10 mg/kg and ≥1.5 mg/kg in rats and dogs, respectively. These dose levels resulted in systemic exposures approximately 0.5 and 0.02 times, respectively, the human exposure (AUC₀₋₂₄ₕ) at the dose of 200 mg daily. There was a trend toward reversibility of these findings 4 weeks after dosing was stopped.

### Abiraterone Acetate

A two-year carcinogenicity study was conducted in rats at oral abiraterone acetate doses of 5, 15, and 50 mg/kg/day for males and 15, 50, and 150 mg/kg/day for females. Abiraterone acetate increased the combined incidence of interstitial cell adenomas and carcinomas in the testes at all dose levels tested. This finding is considered to be related to the pharmacological activity of abiraterone. Rats are regarded as more sensitive than humans to developing interstitial cell tumors in the testes. Abiraterone acetate was not carcinogenic in female rats at exposure levels up to 0.8 times the human clinical exposure (1,000 mg daily) based on AUC. Abiraterone acetate was not carcinogenic in a 6-month study in the transgenic (Tg.rasH2) mouse.

Abiraterone acetate and abiraterone were not mutagenic in an in vitro microbial mutagenesis (Ames) assay or clastogenic in an in vitro cytogenetic assay using primary human lymphocytes or an in vivo rat micronucleus assay.

In repeat-dose toxicity studies in male rats (13- and 26-weeks) and monkeys (39-weeks), atrophy, aspermia/hypospermia, and hyperplasia in the reproductive system were observed at ≥50 mg/kg/day in rats and ≥250 mg/kg/day in monkeys and were consistent with the antiandrogenic pharmacological activity of abiraterone. These effects were observed in rats at systemic exposures similar to humans and in monkeys at exposures approximately 0.6 times the AUC in humans at 1,000 mg daily.

In a fertility study in male rats, reduced organ weights of the reproductive system, sperm counts, sperm motility, altered sperm morphology and decreased fertility were observed in animals dosed for 4 weeks at ≥30 mg/kg/day orally. Mating of untreated females with males that received 30 mg/kg/day oral abiraterone acetate resulted in a reduced number of corpora lutea, implantations and live embryos and an increased incidence of pre-implantation loss. Effects on male rats were reversible after 16 weeks from the last abiraterone acetate administration.

In a fertility study in female rats, animals dosed orally for 2 weeks until day 7 of pregnancy at ≥30 mg/kg/day had an increased incidence of irregular or extended estrous cycles and pre-implantation loss (300 mg/kg/day). There were no differences in mating, fertility, and litter parameters in female rats that received abiraterone acetate. Effects on female rats were reversible after 4 weeks from the last abiraterone acetate administration.

The dose of 30 mg/kg/day in rats is approximately 0.3 times the recommended dose of 1,000 mg/day based on body surface area.

In 13- and 26-week studies in rats and 13- and 39-week studies in monkeys, a reduction in circulating testosterone levels occurred with abiraterone acetate at approximately one half the human clinical exposure based on AUC. As a result, decreases in organ weights and toxicities were observed in the male and female reproductive system, adrenal glands, liver, pituitary (rats only), and male mammary glands. The changes in the reproductive organs are consistent with the antiandrogenic pharmacological activity of abiraterone acetate.

13.2 Animal Toxicology and/or Pharmacology

### Niraparib

In vitro, niraparib bound to DAT, NET, and SERT and inhibited uptake of norepinephrine and dopamine in cells with IC₅₀ values that were lower than the Cₘᵢₙ at steady-state in patients receiving the 200 mg dose. Niraparib has the potential to cause effects in patients related to inhibition of these transporters (e.g., cardiovascular, central nervous system). Intravenous administration of niraparib to vagotomized dogs over 30 minutes at 1, 3, and 10 mg/kg resulted in an increased range of arterial pressures of 13% to 20%, 18% to 27%, and 19% to 25%, respectively, and increased range of heart rates of 2% to 11%, 4% to 17%, and 12% to 21%, respectively, above pre-dose levels. The unbound plasma concentrations of niraparib in dogs at these dose levels were approximately 1.2, 3.9, and 15.5 times the unbound Cₘₐₓ at steady state in patients receiving the 200 mg dose.

In addition, niraparib crossed the blood-brain barrier in rats and monkeys following oral administration. The cerebrospinal fluid plasma Cₘₐₓ ratios of niraparib administered at 10 mg/kg orally to two rhesus monkeys were 0.10 and 0.52.

### Abiraterone Acetate

A dose-dependent increase in cataracts was observed in rats after daily oral abiraterone acetate administration for 26 weeks starting at 350 mg/kg/day (similar to the human clinical exposure (AUC) at 1,000 mg dose daily). In a 39-week monkey study with daily oral abiraterone acetate administration, no cataracts were observed at higher doses (2 times greater than the clinical exposure (AUC) at 1,000 mg dose daily).

### 14 Clinical Studies

14.1 BRCA2-mutated Metastatic Castration-Sensitive Prostate Cancer (mCSPC)

The efficacy of AKEEGA was investigated in AMPLITUDE (NCT04497844), a randomized double-blind, placebo-controlled, multi-cohort, multi-center study in which 696 patients with homologous recombination repair (HRR) gene-mutated (HRRm) mCSPC were randomized (1:1) to receive niraparib 200 mg and abiraterone acetate 1,000 mg (N=348) or placebo and abiraterone acetate (N=348). All patients received prednisone 5 mg daily and were required to have androgen deprivation therapy (ADT) (medical or surgical)>14 days prior to randomization. The only allowable prior systemic therapy in the mCSPC setting, was up to 45 days of abiraterone acetate, up to 6 cycles of docetaxel, and up to 6 months of ADT.

Randomization was stratified by HRR gene alteration (BRCA2 versus CDK12 versus all other pathogenic alterations), prior docetaxel use (yes versus no), and volume of disease at screening (high versus low).

Of the 696 patients enrolled, 323 were randomized as having BRCA2 gene mutation (BRCA2m). Mutation status was determined prospectively using the Foundation One CDx tissue assay or other clinical trial assays.

Among the 323 patients with BRCA2m the median age was 66 years (range 41; 92); 68% were White, 25% Asian, 4% Black, and 3% other or not reported; 10% were Hispanic or Latino; and baseline ECOG performance status was 0 (68%), 1 (30%) or 2 (1.2%). 16% had received prior docetaxel and 11% received prior abiraterone acetate for up to 45 days for mCSPC. 40% had bone-only metastases and 15% had visceral metastases, 10% had BRCA2 mutations in combination with mutations in other HRR genes.

The major efficacy outcome measure was radiographic progression free survival (rPFS) determined by investigator-assessed radiographic progression by bone scan (according to PCWG3 criteria) or soft tissue lesions by CT or MRI (according to RECIST 1.1 criteria) or death, whichever occurred first. Overall Survival (OS) and Time to Symptomatic Progression (TSP) were additional efficacy outcome measures. A statistically significant improvement in rPFS for niraparib and abiraterone acetate compared to placebo and abiraterone acetate was observed in the overall population of patients with HRRm. In an exploratory analysis in the subgroup of 373 patients with non-BRCA2 mutations, the investigator-assessed rPFS hazard ratio was 0.88 (95% CI: 0.63, 1.24), indicating that the improvement in the overall population was primarily attributed to the results seen in the subgroup of patients with BRCA2 mutation.

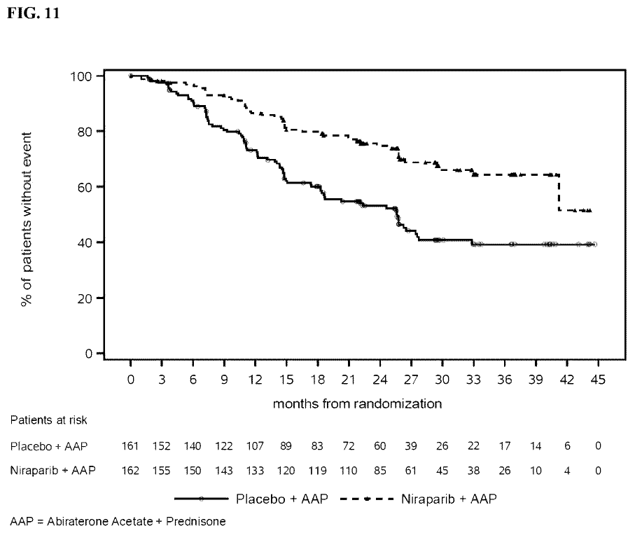

The efficacy results are presented in Table 6 and FIG. 11 for patients with BRCA2 mutations in AMPLITUDE.

TABLE 6
Efficacy Results from the BRCA2m
Subgroup of the AMPLITUDE Study

| | | |
|---|---|---|
| | | Placebo + |
| | | Abiraterone |
| | AKEEGA | Acetate |
| Endpoints | (N = 162) | (N = 161) |

Radiographic Progression-free Survival^{a}

| | | |
|---|---|---|
| Events | 48 (30%) | 82(51%) |
| Median (95% CI) time to event (months) | NE (41, NE) | 26 (18, 28) |

| | |
|---|---|
| Hazard Ratio (95% CI)^{b} | 0.46 (0.32, 0.66) |

^{a}Investigator-assessed
^{b}Calculated using an unstratified Cox proportional hazards model
NE = not estimable

At the first interim analysis for OS, 91 deaths occurred in the BRCA2m population, 36 [22%] in the AKEEGA arm) and 55 [34%] in the placebo and abiraterone acetate arm.

Treatment with AKEEGA resulted in a delay in TSP (HR=0.41, 95% CI=0.26, 0.65). TSP was defined as the time from randomization to the time of symptomatic progression, which included use of external beam radiation for skeletal or pelvic symptoms, cancer-related morbid events, initiation of new systemic anti-cancer therapy, and other cancer-related procedures.

14.2 BRCA-mutated Metastatic Castration-Resistant Prostate Cancer (mCRPC)

The efficacy of AKEEGA was investigated in Cohort 1 of MAGNITUDE (NCT03748641), a randomized double-blind, placebo-controlled, multi-cohort, multi-center study in which 423 patients with homologous recombination repair (HRR) gene-mutated (HRRm) mCRPC were randomized (1:1) to receive niraparib 200 mg and abiraterone 1,000 mg (N=212) or placebo and abiraterone (N=211) until unacceptable toxicity or progression. All patients received prednisone 10 mg daily and a GnRH analog or had prior bilateral orchiectomy. Patients with mCRPC who had not received prior systemic therapy in the mCRPC setting except for a short duration of prior abiraterone acetate plus prednisone (up to four months) and ongoing ADT, were eligible. Patients could have received prior docetaxel or androgen-receptor (AR) targeted therapies in either the metastatic castration-sensitive prostate cancer (mCSPC) or non-metastatic castration-resistant prostate cancer (nmCRPC) setting.

Randomization was stratified by prior docetaxel for mCSPC (yes or no), prior AR targeted therapy for mCSPC or nmCRPC (yes or no), prior abiraterone acetate for mCRPC (yes or no), and BRCA-status (BRCAm vs. other).

Of the 423 patients enrolled, 225 (53%) had BRCA gene mutations (BRCAm). Mutation status of BRCA genes was determined prospectively using the Foundation One CDx tissue assay or other clinical trial assays.

Among the 225 patients with BRCAm, the median age was 68 years (range 43-100) and 66% were 65 years of age or older; 72% were White, 17% Asian, and 1% Black, and 10% other or not reported; 12% were Hispanic or Latino; and baseline ECOG performance status (PS) was 0 (66%) or 1 (34%). Twenty-four percent had received prior docetaxel, 5% received prior AR-targeted therapy for mCSPC or nmCRPC, and 26% received prior abiraterone acetate plus prednisone for up to 4 months for mCRPC. Thirty-seven percent had bone-only metastases and 21% had visceral metastases. Seven percent had BRCA1 mutations, 78% had BRCA2 mutations, and 15% had BRCA mutations in combination with mutations in other HRR genes.

The major efficacy outcome measure was radiographic progression free survival (rPFS) determined by blinded independent central radiology (BICR) review evaluated per Response Evaluation Criteria In Solid Tumors (RECIST) 1.1 (soft tissue lesions) and Prostate Cancer Working Group-3 (PCWG-3) criteria (bone lesions). Overall survival (OS) was an additional efficacy outcome measure.

A statistically significant improvement in rPFS for niraparib plus abiraterone compared to placebo plus abiraterone was observed in BRCAm patients, and the Cohort 1 intention to treat (ITT) population. In an exploratory analysis in the subgroup of 198 (47%) patients with non-BRCA mutations, the rPFS hazard ratio was 0.99 (95% CI: 0.67, 1.44) and the OS hazard ratio was 1.13 (95% CI: 0.77, 1.64), indicating that the improvement in the ITT population was primarily attributed to the results seen in the subgroup of patients with BRCAm.

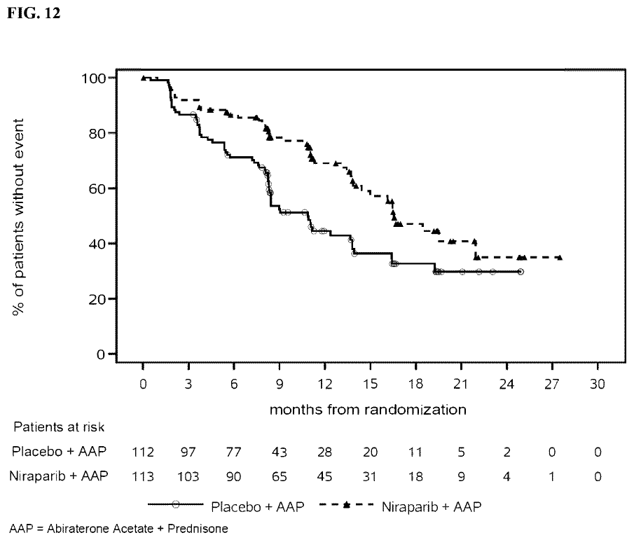

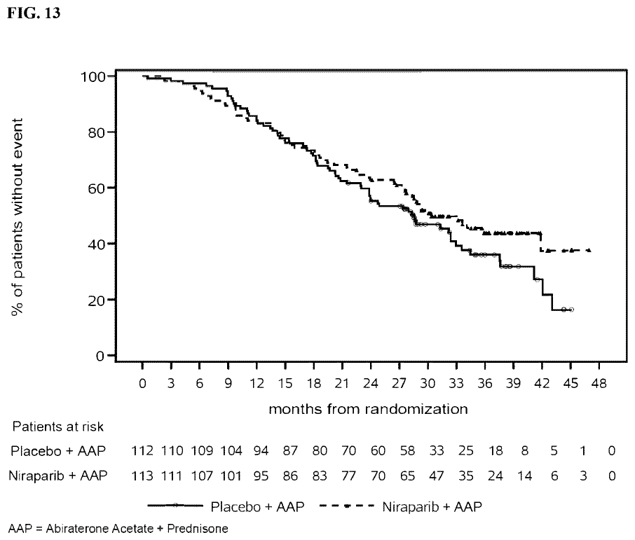

The efficacy results are presented in Table 7 and FIGS. 12 and 13 for patients in Cohort 1 with BRCA mutations.

TABLE 7
Efficacy Results from the BRCAm
Subgroup of the MAGNITUDE Study

| | | |
|---|---|---|
| | | Placebo + |
| | | Abiraterone |
| | AKEEGA | Acetate |
| Endpoints | (N = 113) | (N = 112) |

Radiographic Progression-free Survival^{a}

| | | |
|---|---|---|
| Event of disease | 45 (40%) | 64 (57%) |
| progression or death (%) | | |
| Median, months^{b} (95% CI) | 16.6 (13.9, NE) | 10.9 (8.3, 13.8) |

| | |
|---|---|
| Hazard Ratio (95% CI) | 0.53 (0.36, 0.79) |
| p-value^{c} | 0.0014 |

^{a}rPFS results based on blinded independent central review at primary analysis.
^{b}Cox proportional hazards model stratified by prior docetaxel (yes vs. no) and prior abiraterone (yes vs. no).
^{c}Stratified log-rank test two-sided p-value
NE = not estimable

At the protocol pre-specified final OS analysis in Cohort 1, 60 (53%) deaths and 70 (63%) deaths were observed in the AKEEGA arm and placebo arm, respectively, for patients with BRCAm. In an exploratory OS analysis in the subgroup of patients with BRCAm, the median in the AKEEGA arm was 30.4 (95% CI: 27.6, NE) and 28.6 months (95% CI: 23.8, 33.0) in the placebo arm, with an OS hazard ratio of 0.79 (95% CI: 0.55, 1.12).

### 16 how Supplied/Storage and Handling

AKEEGA® (niraparib and abiraterone acetate) tablets are available in the strengths and packages listed below:
AKEEGA 50 mg/500 mg film-coated tablets
Yellowish orange to yellowish brown, oval, film-coated tablets debossed with “N 50 A” on one side and plain on the other side. They are available in bottles of 60 tablets.
NDC 57894-050-60
AKEEGA 100 mg/500 mg film-coated tablets
Orange, oval, film-coated tablets debossed with “N 100 A” on one side and plain on the other side. They are available in bottles of 60 tablets.
NDC 57894-100-60

### Storage and Handling

Store at 20° C. to 25° C. (68° F. to 77° F.); excursions permitted to 15° C. to 30° C. (59° F. to 86° F.) [see USP Controlled Room Temperature].

Based on its mechanism of action, AKEEGA may harm a developing fetus. Females who are or may become pregnant should handle AKEEGA tablets with protection, e.g., gloves [see Use in Specific Populations (8.1)].

### 17 Patient Counseling Information

Advise the patient to read the FDA-approved patient labeling (Patient Information).

### Hematologic Adverse Reactions

Advise patients that periodic monitoring of their blood counts is recommended. Advise patients to contact their healthcare provider for new onset of pallor, weakness, dyspnea, fatigue, bleeding, fever, or symptoms of infection [see Warnings and Precautions (5.1, 5.2)].

### Hypokalemia, Fluid Retention, and Cardiovascular Adverse Reactions

Inform patients that AKEEGA is associated with, hypokalemia that may lead to QT prolongation. Advise patients that hypertension, hypokalemia, and fluid retention will be monitored at least weekly for the first two months, then once a month. Advise patients to adhere to corticosteroids and to report symptoms of hypokalemia or edema to their healthcare provider [see Warnings and Precautions (5.3)].

### Hepatotoxicity and Hepatic Impairment

Inform patients that AKEEGA is associated with severe hepatotoxicity. Inform patients that their liver function will be monitored using blood tests. Advise patients to immediately report symptoms of hepatotoxicity to their healthcare provider [see Warnings and Precautions (5.4)].

### Adrenocortical Insufficiency

Inform patients that AKEEGA with prednisone is associated with adrenal insufficiency. Advise patients to report symptoms of adrenocortical insufficiency to their healthcare provider [see Warnings and Precautions (5.5)].

### Hypoglycemia

Inform patients that AKEEGA is associated with hypoglycemia. Advise patients with diabetes to monitor blood glucose during and after discontinuation of treatment with AKEEGA [see Warnings and Precautions (5.6)].

### Posterior Reversible Encephalopathy Syndrome

Inform patients that they are at risk of developing posterior reversible encephalopathy syndrome (PRES) that can present with signs and symptoms including seizure, headaches, altered mental status, or vision changes. Advise patients to contact their healthcare provider if they develop any of these signs or symptoms [see Warnings and Precautions (5.8)].

### Dosage and Administration

Inform patients that AKEEGA is taken orally once daily with prednisone daily (according to their healthcare provider's instructions) and to not interrupt or stop either of these medications without consulting their healthcare provider [see Dosage and Administration (2.2)].
Inform patients coadministered a gonadotropin-releasing hormone (GnRH) analog therapy that they need to maintain this treatment during the course of treatment with AKEEGA [see Dosage and Administration (2.2)].
Inform patients that in the event of a missed daily dose of AKEEGA, they should take their normal dose as soon as possible on the same day and resume their next dose at the normal schedule on the following day. The patient should not take extra tablets to make up the missed dose [see Dosage and Administration (2.2)].
Instruct patients to take AKEEGA tablets as a single dose once daily on an empty stomach. Instruct patients to take AKEEGA on an empty stomach at least one hour before or two hours after food. AKEEGA taken with food causes increased exposure and may result in adverse reactions. Instruct patients to swallow tablets whole with water and not to break, crush, or chew the tablets [see Dosage and Administration (2.2)].

### Embryo-Fetal Toxicity

Inform patients that AKEEGA may harm a developing fetus and can cause loss of pregnancy [see Warnings and Precautions (5.9) and Use in Specific Populations (8.1)].
Advise males with female partners of reproductive potential to use effective contraception during treatment and for 4 months after the last dose of AKEEGA [see Use in Specific Populations (8.3)].
Advise females who are pregnant or may become pregnant to handle AKEEGA tablets with protection, e.g., gloves [see Use in Specific Populations (8.1) and How Supplied/Storage and Handling (16)].

### Infertility

Advise male patients that AKEEGA may impair fertility [see Use in Specific Populations (8.3)].

### Product of France

Manufactured for:
Janssen Biotech, Inc.
Horsham, PA 19044, USA
For patent information: www.janssenpatents.com
C Johnson & Johnson and its affiliates 2025

PATIENT INFORMATION
AKEEGA ® (a kee' gah)
(niraparib and abiraterone acetate)
tablets

What is the most important information I should know about AKEEGA?
AKEEGA may cause serious side effects including:
Bone marrow problems called myelodysplastic syndrome (MDS) or a type of cancer of the blood called acute
myeloid leukemia (AML). MDS or AML that may lead to death has happened in people treated with AKEEGA. If
you develop MDS or AML, your healthcare provider will stop treatment with AKEEGA.
Symptoms of low blood cell counts (low red blood cells, low white blood cells, and low platelets) are common during
treatment with AKEEGA but can also be a sign of serious bone marrow problems, including MDS and AML. Tell
your healthcare provider if you have any of the following symptoms during treatment with AKEEGA:

| | |
|---|---|
| pale skin | fever |
| weakness | frequent infections |
| shortness of breath | blood in urine or stool |
| feeling tired | weight loss |
| bruising or bleeding more easily | |

Your healthcare provider will do blood tests to check your blood cell counts:
weekly during the first month of treatment,
every 2 weeks for the next 2 months of treatment,
monthly for the remainder of the year,
then every other month and as needed during treatment with AKEEGA.
See “What are the possible side effects of AKEEGA?” for more information about side effects.
What is AKEEGA?
AKEEGA is a prescription medicine used with prednisone to treat adults with prostate cancer:
who have a certain type of abnormal BRCA gene, and
whose prostate cancer has spread to other parts of the body (metastatic prostate cancer).
Your healthcare provider will perform a test to make sure AKEEGA is right for you.
It is not known if AKEEGA is safe and effective in females.
It is not known if AKEEGA is safe and effective in children.
Before taking AKEEGA, tell your healthcare provider about all of your medical conditions, including if you:
have high blood pressure or heart problems
have low blood potassium levels
have liver or kidney problems
have a history of adrenal problems
have diabetes
are receiving any other treatment for prostate cancer
are pregnant or plan to become pregnant. AKEEGA can cause harm to your unborn baby and loss of pregnancy
(miscarriage). Females who are or may become pregnant should handle AKEEGA tablets with protection, such as
gloves.
have a partner who is pregnant or may become pregnant.
Males with female partners who are able to become pregnant should use effective birth control (contraception) during
treatment and for 4 months after the last dose of AKEEGA.
are breastfeeding or plan to breastfeed. It is not known if AKEEGA passes into your breastmilk.
Tell your healthcare provider about all the medicines you take, including prescription and over-the-counter
medicines, vitamins, and herbal supplements. AKEEGA may affect the way other medicines work, and other medicines
may affect how AKEEGA works.
How should I take AKEEGA?
Take AKEEGA and prednisone exactly as your healthcare provider tells you.
Your healthcare provider may change your dose, temporarily stop, or permanently stop treatment with AKEEGA if
you have certain side effects.
Do not change or stop taking your prescribed dose of AKEEGA or prednisone without talking with your
healthcare provider first.
Take your prescribed dose of AKEEGA 1 time a day.
Take AKEEGA on an empty stomach at least 1 hour before or 2 hours after food. Taking AKEEGA with food may
cause more of the medicine to be absorbed by the body than is needed and this may cause side effects.
Swallow AKEEGA tablets whole with water. Do not break, crush, or chew tablets.
If you miss a dose of AKEEGA, take the dose as soon as possible on the same day. Return to your normal schedule on
the following day. Do not take extra tablets to make up the missed dose.
You should start or continue a gonadotropin-releasing hormone (GnRH) analog therapy during your treatment with
AKEEGA unless you have had a surgery to lower the amount of testosterone in your body (surgical castration).
If you take too much AKEEGA, call your healthcare provider or go to the nearest hospital emergency room right away.
What are the possible side effects of AKEEGA?
AKEEGA may cause serious side effects, including:
See “What is the most important information I should know about AKEEGA?”
Low blood potassium levels (hypokalemia), fluid retention (edema), high blood pressure (hypertension) and
heart problems. To decrease the chance of this happening, you must take prednisone with AKEEGA exactly as your
healthcare provider tells you. Your healthcare provider will check your blood pressure, do blood tests to check your
potassium levels, and check for any signs and symptoms of fluid retention at least weekly for the first 2 months of
treatment, then 1 time a month during treatment with AKEEGA. Tell your healthcare provider if you have any of the
following symptoms:

| | |
|---|---|
| dizziness | confusion |
| fast or irregular heartbeats | muscle weakness |
| feel faint or lightheaded | pain in your legs |
| headache | swelling in your hands, ankles, legs or feet |

Liver problems. Severe liver problems, liver failure and death has happened in people treated with abiraterone acetate,
one of the medicines in AKEEGA. Your healthcare provider will do blood tests to check your liver function before
starting treatment with AKEEGA, every 2 weeks for the first 3 months of treatment, and then monthly thereafter during
treatment with AKEEGA. Tell your healthcare provider right away if you develop any symptoms of liver problems,
including:

| | |
|---|---|
| yellowing of the skin or eyes | severe nausea or vomiting |
| darkening of the urine | |

Adrenal problems. Adrenal problems may happen if you stop taking prednisone, get an infection, or are under stress.
Tell your healthcare provider right away if you develop any symptoms of adrenal problems, including:

| | |
|---|---|
| feeling tired | nausea or vomiting |
| weakness | weight loss |
| feeling dizzy or lightheaded | |

Low blood sugar (hypoglycemia). AKEEGA may cause low blood sugar in people taking medicines for diabetes.
Severe low blood sugar has happened in people who take certain medicines for diabetes and were treated with
abiraterone acetate, one of the medicines in AKEEGA. You and your healthcare provider should check your blood
sugar levels during treatment and after you stop treatment with AKEEGA. Your healthcare provider may need to
change the dose of your diabetes medicine to decrease your risk of low blood sugar. Tell your healthcare provider
right away if you have any of the following signs or symptoms of low blood sugar, including:
headache
drowsiness
weakness
dizziness
confusion
Increased risk of bone fracture and death when abiraterone acetate, one of the medicines in AKEEGA, and
prednisone or prednisolone is used in combination with a type of radiation called Radium 223 (Ra-223) dichloride.
You should not receive treatment with Ra-223 dichloride for at least 5 days after your last dose of AKEEGA with
prednisone. Tell your healthcare provider about any other treatments you are taking for prostate cancer.
Posterior Reversible Encephalopathy Syndrome (PRES). PRES is a condition that affects the brain and may
happen during treatment with AKEEGA. If you have headache, vision changes, confusion, or seizure with or without
high blood pressure, please contact your healthcare provider.
The most common side effects of AKEEGA include:

| | | |
|---|---|---|
| decreased hemoglobin | constipation | decreased potassium level in the blood |
| decreased lymphocytes | high blood pressure | swelling in your legs or feet |
| muscle and bone pain | nausea | increased bilirubin levels in the blood |
| tiredness | decreased neutrophils | respiratory tract infection |
| decreased platelets | changes in kidney function blood tests | irregular heartbeat (arrhythmia) |
| changes in liver function blood tests | increased potassium level in the blood | |

AKEEGA may cause fertility problems in males, which may affect the ability to father children. Talk to your healthcare
provider if you have concerns about fertility.
These are not all the possible side effects of AKEEGA.
Call your doctor for medical advice about side effects. You may report side effects to FDA at 1-800-FDA-1088.
How should I store AKEEGA?
Store AKEEGA at room temperature between 68° F. to 77° F. (20° C. to 25° C.).
Keep AKEEGA and all medicines out of the reach of children.
General information about the safe and effective use of AKEEGA.
Medicines are sometimes prescribed for purposes other than those listed in a Patient Information leaflet. Do not use
AKEEGA for a condition for which it was not prescribed. Do not give AKEEGA to other people, even if they have the
same symptoms that you have. It may harm them. You can ask your healthcare provider or pharmacist for information
about AKEEGA that is written for health professionals.
What are the ingredients in AKEEGA?
Active ingredients: niraparib tosylate and abiraterone acetate
Inactive ingredients:
Core tablet: colloidal anhydrous silica, crospovidone, hypromellose, lactose monohydrate, magnesium stearate, silicified
microcrystalline cellulose, sodium lauryl sulfate.
50 mg/500 mg film-coated tablets: The film-coating contains iron oxide black, iron oxide red, iron oxide yellow, sodium
lauryl sulphate, glycerol monocaprylocaprate, polyvinyl alcohol, talc, and titanium dioxide.
100 mg/500 mg film-coated tablets: The film-coating contains iron oxide red, iron oxide yellow, sodium lauryl sulphate,
glycerol monocaprylocaprate, polyvinyl alcohol, talc, and titanium dioxide.
Manufactured for: Janssen Biotech, Inc., Horsham, PA 19044, USA
For patent information: www.janssenpatents.com
©Johnson & Johnson and its affiliates 2025
For more information, call Janssen Biotech, Inc. at 1-800-526-7736 (1-800-JANSSEN) or go to www.akeegahcp.com.

This Patient Information has been approved by the U.S. Food and Drug Administration Revised: 12/2025

1. A method of improving the median radiographic progression-free survival (rPFS) in a patient with metastatic castration-sensitive prostate cancer (mCSPC), who is positive for germline and/or somatic homologous recombination repair (HRR) gene alterations; said method comprising administering to said patient a daily dose of 200 mg of niraparib, 1000 mg of abiraterone acetate, and 5 mg of prednisone or prednisolone; wherein the HRR gene alterations are selected from the genes BRCA2 or CHEK2, and wherein the rPFS is improved as indicated by a hazard ratio of less than 0.60 compared to a treatment with 1000 mg of abiraterone acetate and 5 mg of prednisone or prednisolone.

2. The method of improving the median rPFS according to claim 1, wherein the patient has a BRCA2 alteration and said median rPFS is improved as indicated by a hazard ratio of 0.451 compared to a treatment with 1000 mg of abiraterone acetate and 5 mg of prednisone or prednisolone.

3. The method of improving the median rPFS according to claim 2, wherein the median rPFS is more than 41 months, compared to 25.6 months for a treatment with 1000 mg of abiraterone acetate and 5 mg of prednisone or prednisolone.

4. The method of improving the median rPFS according to claim 1, wherein the patient has a CHEK2 alteration and said median rPFS is improved as indicated by a hazard ratio of 0.550 compared to a treatment with 1000 mg of abiraterone acetate and 5 mg of prednisone or prednisolone.

5. The method of improving the median rPFS according to claim 4, wherein the median rPFS is of 47.87 months, compared to 30.55 months for a treatment with 1000 mg of abiraterone acetate and 5 mg of prednisone or prednisolone.

6. A method of improving the median time to symptomatic progression (TSP) in a patient with metastatic castration-sensitive prostate cancer (mCSPC), who is positive for germline and/or somatic HRR gene alterations; said method comprising administering to said patient a daily dose of 200 mg of niraparib, 1000 mg of abiraterone acetate, and 5 mg of prednisone or prednisolone; wherein the HRR gene alterations are selected from non-BRCA genes defined as single or co-occurring alterations in BRIP1, PALB2, RAD51B, and RAD54L and excluding those with BRCA co-occurring.

7. The method of improving the median TSP according to claim 6, wherein said median TSP is improved as indicated by a hazard ratio of less than 0.60 compared to a treatment with 1000 mg of abiraterone acetate and 5 mg of prednisone or prednisolone, preferably wherein the TSP is improved as indicated by a hazard ratio of 0.58 (95% CI: 0.37,0.93; nominal p-value=0.022).

8. A method of improving the median overall survival (OS) in a patient with metastatic castration-sensitive prostate cancer (mCSPC), who is positive for germline and/or somatic BRCA2 gene alterations; said method comprising administering to said patient a daily dose of 200 mg of niraparib, 1000 mg of abiraterone acetate, and 5 mg of prednisone or prednisolone.

9. A method of improving time to subsequent therapy (TST) in a patient with metastatic castration-sensitive prostate cancer (mCSPC), who is positive for germline and/or somatic HRR gene alterations; said method comprising administering to said patient a daily dose of 200 mg of niraparib, 1000 mg of abiraterone acetate, and 5 mg of prednisone or prednisolone; wherein the HRR gene alterations are selected from the genes BRCA1, BRCA2, BRIP1, CDK12, CHEK2, FANCA, PALB2, RAD51B, and RAD54L.

10. The method of improving the TST according to claim 9, wherein said TST is improved as indicated by a hazard ratio of 0.52 (95% CI: 0.40,0.66, nominal p<0.0001) compared to a treatment with 1000 mg of abiraterone acetate and 5 mg of prednisone or prednisolone.

11. (canceled)

12. The method of improving the TST according to claim 9, wherein the HRR gene alterations are selected from the genes BRCA1, BRCA2, BRIP1, PALB2, RAD51B, and RAD54L and wherein said TST is improved as indicated by a hazard ratio of 0.471 (95% CI: 0.35,0.64, nominal p-value<0.0001) compared to a treatment with 1000 mg of abiraterone acetate and 5 mg of prednisone or prednisolone.

13. (canceled)

14. The method of improving the TST according to claim 9, wherein the HRR gene alterations are selected from the genes BRCA1 and BRCA2 and wherein said TST is improved as indicated by a hazard ratio of 0.41 (95% CI: 0.30,0.57, nominal p<0.0001) compared to a treatment with 1000 mg of abiraterone acetate and 5 mg of prednisone or prednisolone.

15. The method according to claim 1, wherein the method comprises administering, to said patient, androgen deprivation therapy (ADT) in combination with a daily dose of 200 mg of niraparib, 1000 mg of abiraterone acetate, and 5 mg of prednisone or prednisolone.

16. The method according to claim 1, wherein the patient has received and continues receiving androgen deprivation therapy (ADT).

17. The method according to claim 1, wherein the patient has received prior docetaxel treatment.

18. The method according to claim 1, wherein the patient has;
an Eastern Cooperative Oncology Group Performance Status (ECOG PS) Grade <2;
an absolute neutrophil count ≥1.5×10⁹/L;
hemoglobin ≥9.0 g/dL;
platelet count ≥100×10⁹/L;
creatinine <2× upper limit of normal (ULN);
serum potassium ≥3.5 mmol/L;
serum total bilirubin ≤1.5× ULN or direct bilirubin ≤1× ULN; and/or
AST or ALT ≤3× ULN.

19.-25. (canceled)

26. The method according to claim 1, wherein the patient has not received prior treatment with a PARP inhibitor.

27. The method according to claim 1, wherein the patient has not received prior androgen receptor-targeted therapy, immunotherapy, or radiopharmaceutical agents for prostate cancer.

28. The method of claim 27, wherein the androgen receptor-targeted therapy is selected from apalutamide, enzalutamide, and darolutamide.

29. The method according to claim 1, wherein niraparib is administered as niraparib tosylate monohydrate.

30. The method according to claim 1, wherein niraparib and abiraterone acetate are administered as a drug product or fixed-dose combination (FDC).

31. The method according to claim 1, wherein niraparib and abiraterone acetate are administered as a free-dose combination (FrDC).

32.-33. (canceled)
